(19) United States
(12) Patent Application Publication
(10) Pub. No.: US 2025/0339508 A1
(43) Pub. Date: Nov. 6, 2025

(54) THERAPEUTIC INTERFERING PARTICLES FOR CORONA VIRUS

(71) Applicants: THE J. DAVID GLADSTONE INSTITUTES, A TESTAMENTARY TRUST ESTABLISHED UNDER THE WILL OF J. DAVID GLAD, San Francisco, CA (US); VxBiosciences, Inc., Berkeley, CA (US)

(72) Inventors: Robert RODICK, Berkeley, CA (US); Leor S. WEINBERGER, San Francisco, CA (US); Sonali CHATURVEDI, San Francisco, CA (US)

(21) Appl. No.: 18/553,900

(22) PCT Filed: Apr. 25, 2022
(86) PCT No.: PCT/US2022/026223

§ 371 Date: Oct. 4, 2023

(30) Foreign Application Priority Data
Apr. 23, 2021 (WO) PCT/US2021/028809

(51) Int. Cl. A61K 39/215; A61K 39/00; A61P 37/04; C12N 15/86

(52) U.S. Cl. CPC A61K 39/215; A61P 37/04; C12N 15/86; A61K 2039/5258; A61K 2039/53; C12N 2770/20023; C12N 2770/20034; C12N 2770/20043; C12N 2770/20071

(57) ABSTRACT

Described herein are compositions of recombinant SARS-CoV-2 constructs and particles that can interfere with or block infection of uninfected cells. The compositions and methods described herein are useful for treatment of SARS-Co V-2 infections. The recombinant SARS-CoV-2 construct cannot replicate by itself, but can replicate in the presence of infective SARS-CoV-2 (e.g., replication competent SARS-CoV-2). Thus, the present application in one aspect provides a recombinant SARS-Co V-2 construct (e.g., SARS-CoV-2 TIP) capable of interfering with SARS-CoV-2 replication, wherein the recombinant SARS-CoV-2 construct cannot replicate by itself, and wherein the recombinant SARS-CoV-2 construct can replicate in the presence of SARS-CoV-2.

### CROSS-REFERENCE TO RELATED APPLICATION

This application claims priority benefit from International Application No. PCT/US2021/028809 filed Apr. 23, 2021, entitled “Therapeutic Interfering Particles for Corona Virus,” the contents of which are incorporated herein by reference in their entirety for all purposes.

### GOVERNMENT SUPPORT

This invention was made with government support under 1-DP2-OD006677-01 awarded by the National Institutes of Health and under D17AC00009 awarded by DOD/DARPA. The government has certain rights in the invention.

### SEQUENCE STATEMENT

The content of the following submission on ASCII text file is incorporated herein by reference in its entirety: a computer readable form (CRF) of the Sequence Listing (file name: 210272000141SEQLIST.TXT, date recorded: Apr. 24, 2022, size: 145,769 bytes).

### FIELD OF THE INVENTION

The present invention relates in some aspects to therapeutic interfering particles for the treatment of viral infections, such as infections caused by SARS-CoV-2.

### BACKGROUND

The World Health Organization has declared Covid-19 a global pandemic. A highly infectious coronavirus, officially called SARS-CoV-2, causes the Covid-19 disease. Even with the most effective containment strategies, the spread of the Covid-19 respiratory disease has only been slowed. While effective vaccines exist for current strain of SARS-CoV-2, new variants and mutant strains continue to develop. Hence, there is a need for treatments that interfere with infection as well and/or new vaccines that can facilitate recovery from infection and put an end to the SARS-CoV-2 pandemic.

### BRIEF SUMMARY

Provided are compositions comprising recombinant SARS-CoV-2 constructs, such as therapeutic interfering particles (e.g., TIPs), that can interfere with or block infection of uninfected cells. The compositions are useful for the prevention and treatment of SARS-CoV-2 infections.

One aspect of the present application provides a recombinant SARS-CoV-2 construct capable of interfering with SARS-CoV-2 replication, comprising: (a) a 5′UTR region comprising at least 100 nucleotides of a SARS-Cov-2 5′UTR or a variant thereof, (b) an intervening sequence, and (c) a 3′UTR region comprising at least 100 nucleotides of a SARS-Cov-2 3′UTR or a variant thereof, wherein the recombinant SARS-CoV-2 construct cannot replicate by itself, wherein the recombinant SARS-CoV-2 construct can replicate in the presence of SARS-CoV-2, and wherein the intervening sequence is about 1 base pair (bp) to about 29000 bp (including for example about 1 bp to about 5000 bp, about 1 bp to about 500 bp).

In some embodiments, the total length of the 5′UTR region, the optional intervening sequence, and the 3′UTR region in the recombinant SARS-CoV-2 construct is about 1000 bp to about 10000 bp. In some embodiments, the total length of the 5′UTR region, the optional intervening sequence, and the 3′UTR region in the recombinant SARS-CoV-2 construct is about 2000 bp to about 3500 bp.

In some embodiments, the 5′UTR region comprises nucleotides 1-265 of SEQ ID NO: 1, or a variant thereof. In some embodiments, the 5′UTR region comprises two or more copies of 5′UTR sequences, each comprising at least 100 nucleotides of a SARS-Cov-2 5′UTR or a variant thereof. In some embodiments, the 3′UTR region comprises nucleotides 29675-29870 of SEQ ID NO: 1, or a variant thereof. In some embodiments, the 3′UTR region comprises nucleotides 29675-29903 of SEQ ID NO: 1, or a variant thereof. In some embodiments, the 3′UTR region comprises two or more copies of 3′UTR sequences, each comprising at least 100 nucleotides of a SARS-Cov-2 3′UTR or a variant thereof.

In some embodiments, the recombinant SARS-CoV-2 construct further comprises a packaging signal for SAR-CoV-2. In some embodiments, the packaging signal comprises stem loop 5 in the SARS-CoV-2 5′UTR.

In some embodiments, the intervening sequence comprises a SARS-CoV-2 sequence, a heterologous sequence, or a combination thereof. In some embodiments, the intervening sequence comprises a SARS-CoV-2 sequence. In some embodiments, the SARS-CoV-2 sequence does not encode a functional viral protein.

In some embodiments, the recombinant SARS-CoV-2 construct comprises nucleotides 1-450 of SEQ ID NO: 1, or a variant thereof. In some embodiments, the recombinant SAR-CoV-2 construct comprises nucleotides 1-1540 of SEQ ID NO: 1, or a variant thereof. In some embodiments, the recombinant SARS-CoV-2 construct comprises nucleotides 29543-29903 of SEQ ID NO: 1, or a variant thereof. In some embodiments, the recombinant SARS-CoV-2 construct comprises nucleotides 29543-29870 of SEQ ID NO: 1, or a variant thereof. In some embodiments, the recombinant SARS-CoV-2 construct comprises nucleotides 29191-29903 of SEQ ID NO: 1, or a variant thereof. In some embodiments, the recombinant SARS-CoV-2 construct comprises nucleotides 29191-29870 of SEQ ID NO: 1, or a variant thereof.

In some embodiments, the intervening sequence comprises a heterologous sequence. In some embodiments, the heterologous sequence does not encode a functional protein. In some embodiments, the heterologous sequence encodes one or more functional proteins. In some embodiments, the heterologous sequence encodes a reporter protein. In some embodiments, the heterologous sequence comprises a marker sequence. In some embodiments, the marker sequence is a barcode sequence.

In some embodiments, the recombinant SARS-CoV-2 construct is an mRNA.

In some embodiments, the recombinant SARS-CoV-2 construct comprises a 3′ modification. In some embodiments, the recombinant SARS-CoV-2 construct comprises a 3′ extended sequence. In some embodiments, the 3′ extended sequence is an extended polyA sequence. In some embodiments, the extended polyA sequence comprises at least about 100 adenine nucleotides.

In some embodiments, the recombinant SARS-CoV-2 construct comprises 5′ modification. In some embodiments, the 5′ modification is a 5′ cap. In some embodiments, the 5′ cap is a 5′ methyl cap.

In some embodiments, the recombinant SARS-CoV-2 construct is a DNA. In some embodiments, the recombinant SARS-CoV-2 construct is a vector. In some embodiments, the recombinant SARS-CoV-2 construct comprises a promoter upstream of the 5′UTR region. In some embodiments, the promoter is a T7 promoter. In some embodiments, the recombinant SARS-CoV-2 construct comprises a 3′ extended polyA sequence or a signal for polyA addition.

In some embodiments, the recombinant SARS-CoV-2 construct genomic RNA is produced at a higher rate than SARS-CoV-2 genomic RNA when present in a host cell infected with SARS-CoV-2, such that the ratio of the construct SAR-CoV-2 genomic RNA to the SARS-CoV-2 genomic RNA is greater than 1 in the cell.

In some embodiments, the recombinant SARS-CoV-2 construct has a same transmission frequency compared to SARS-CoV-2. In some embodiments, the recombinant SARS-CoV-2 construct has a lower transmission frequency than SARS-CoV-2. In some embodiments, the recombinant SARS-CoV-2 construct has a higher transmission frequency than SARS-CoV-2.

In some embodiments, the recombinant SARS-CoV-2 construct is packaged with the same or a higher efficiency than SARS-CoV-2 when present in a host cell infected with SARS-CoV-2.

In some embodiments, the recombinant SARS-CoV-2 construct has a basic reproduction ratio (R₀)>1.

In some aspects, provided herein is a viral-like particle comprising any one of the recombinant SARS-CoV-2 constructs described herein and a viral envelope protein.

In some aspects, provided herein is an isolated cell comprising any one of the recombinant SARS-CoV-2 constructs described herein.

In some aspects, provided herein is a pharmaceutical composition comprising any one of the recombinant SARS-CoV-2 constructs described herein and a pharmaceutically acceptable excipient. In some embodiments, the recombinant SARS-CoV-2 construct is present in a delivery vehicle. In some embodiments, the delivery vehicle is a lipid nanoparticle. In some embodiments, the pharmaceutical composition is an aerosol formulation.

In some aspects, provided herein is a method of treating or preventing SARS-CoV-2 infection in an individual, comprising administering to the individual an effective amount of the pharmaceutical composition of any one of the preceding embodiments. In some embodiments, the pharmaceutical composition is administered prior to the individual being infected with SARS-CoV-2. In some embodiments, the pharmaceutical composition is administered after the individual being infected with SARS-CoV-2. In some embodiments, the SARS-CoV-2 is from a SARS-CoV-2 strain selected from B.1.1.7, B.1.351, P.1, or B.1.617.2. In some embodiments, the pharmaceutical composition is administered as a single dose. In some embodiments, the pharmaceutical composition is administered as multiple doses. In some embodiments, the pharmaceutical composition is administered intranasally. In some embodiments, the individual has a medical condition, a pre-existing condition, or a condition that reduces heart, lung, brain, or immune system function. In some embodiments, the individual is immunocompromised. In some embodiments, the individual is a human.

In some aspects, provided herein is a kit for treating or treating or preventing SARS-CoV-2 viral infection in an individual, comprising the pharmaceutical composition of any one of the preceding embodiments and an instruction for carrying out the method of any one of the preceding embodiments.

In some aspects, provided herein is an inhibitor of SARS-CoV-2 transcription regulating sequences (TRSs) that can bind to one or more of: TRS1-L: 5′-cuaaac-3′ (SEQ ID NO:36), TRS2-L: 5′-acgaac-3′ (SEQ ID NO:37), TRS3-L: 5′-cuaaacgaac-3′ (SEQ ID NO:38), or a combination thereof. In some embodiments, the inhibitor comprises a sequence comprising or consisting essentially of: TRS1-ACGAACCUAAACACGAACCUAAAC (SEQ ID NO:25); TRS2-ACGAACACGAACACGAACACGAAC (SEQ ID NO:26); TRS3-CUAAACCUAAACCUAAACCUAAAC (SEQ ID NO:27); or a combination thereof.

In some aspects, provided herein is a pharmaceutical composition comprising the inhibitor of any of the preceding embodiments and a pharmaceutically acceptable excipient.

In some aspects, provided herein is a pharmaceutical composition comprising a pharmaceutically acceptable excipient, (a) an inhibitor of SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), or a combination thereof, and (b) a recombinant SARS-CoV-2 construct, the construct comprising: at least 100 nucleotides of a SARS-CoV-2 5′ untranslated region (5′UTR), at least 100 nucleotides of a SARS-CoV-2 3′ untranslated region (3′UTR), or a combination thereof.

Also provided are kits and articles of manufacture comprising any one of the compositions described above and instructions for any one of the methods described above.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows a schematic diagram of the SARS-CoV-2 genome and encoded open reading frames (ORFs).

FIGS. 2A-2B illustrate infection of cells by wild type and defective SARS-CoV-2. FIG. 2A shows a schematic representation of infection by a wild-type SARS-CoV-2 genome. After integration into a cellular genome (DNA at left), SARS-CoV-2 RNAs are generated that ultimately produce the packaging proteins that form the virus capsid. Infective SARS-CoV-2 can escape their original host cell and infect new cells if they have the needed (functional wild type) surface recognition proteins. FIG. 2B shows a schematic of infection when defective SARS-CoV-2 particles (referred to as Therapeutic Interfering Particles, TIPs) are present with viable SARS-CoV-2. The defective SARS-CoV-2 particles have pared-down versions of the SARS-CoV-2 genome engineered to carry a packaging signal, and other viral cis elements required for packaging. The defective SARS-CoV-2 RNA can thus only be made by cells that also express SARS-CoV-2 proteins. The defective SARS-CoV-2 particles are engineered to produce substantially more defective SARS-CoV-2 genomic RNA copies than wild type SARS-CoV-2 in dually infected cells. With disproportionately more defective SARS-CoV-2 genomic RNA than wild type SARS-CoV-2 genomic RNA, the SARS-CoV-2 packaging materials are mainly wasted enclosing defective SARS-CoV-2 genomic RNA. The defective SARS-CoV-2 particles lower the wild type SARS-CoV-2 burst size and convert infected cells from producing wild type SARS-CoV-2 into producing mostly defective SARS-CoV-2 particles, thereby lowering the wild type SARS-CoV-2 viral load.

FIG. 3 schematically illustrates a method for constructing a randomized, barcoded deletion library for making defective SARS-CoV-2 particles. The schematic cycle method for constructing a barcoded TIP candidate library from a molecular clone involves: [1] in vitro introduction of a retrotransposition into circular SARS-CoV-2 double stranded DNA, [2] exonuclease-mediated excision of the randomly inserted retrotransposon, [3] enzymatic chew back to create a deletion (A) in the circular SARS-CoV-2, and [4] circularizing and barcoding during re-ligation to generate the barcoded TIP candidate library (see, e.g., WO201811225 by Weinberger et al. and WO2014151771 by Weinberger et al., which are both incorporated herein by reference in their entireties). FIG. 4 a schematic diagram illustrating molecular details and steps for one embodiment of a method of generating a deletion library. In step (a) the meganuclease (e.g., 1-Scel or 1-Ceul) cleaves the SARS-CoV-2 double stranded DNA. In step (b) the cleaved ends of the SARS-CoV-2 DNA are chewed back. In step (c), the chewed back ends are repaired. Thus, a deleted gap (A) is present between the ends. In step (d) the 5′ phosphate is removed by alkaline phosphatase (AP) and a dA tail is generated with Klenow. In step (e), the ends are ligated to a barcode cassette, thereby generating numerous circular, barcoded deletion SARS-CoV-2 mutants.

FIGS. 5A-5C illustrate methods for generating and analyzing random deletion libraries of SARS-CoV-2 deletion mutants. FIG. 5A schematically illustrates generation of a random deletion library (RDL) for a 30 kb SARS-CoV-2 molecular clone. Three 10 kb fragments are shown that were used for RDL sub-libraries, where the three fragments were different segments of the SARS-CoV-2 genome. The ends of the three fragments were chewed back (e.g., as described in FIG. 4), and the barcodes (shaded circles) were inserted as the deleted SARS-CoV-2 DNA fragments were ligated. Hence, the barcodes will be at different positions along the fragments. Because the barcodes include sites for primer initiation, sequencing readily identified where the deletions reside in the different SARS-CoV-2 deletion mutants. FIG. 5B graphically illustrates illumina deep sequencing landscapes of barcode positions in the three random deletion sub-libraries. Such sequencing showed that the sub-libraries contain more than 587,000 unique SARS-CoV-2 deletion mutants. FIG. 5C shows gels of electrophoretically separated DNA from the ligated RDL libraries illustrating that there are bands of about 30 kb as well as lower molecular weight bands (ladder is in left lane; the 3 additional lanes are triplicates).

FIGS. 6A-6D illustrate the ‘viroreactor’ strategy used to generate SARS-CoV-2 therapeutic interfering particles (TIPs). FIG. 6A schematically illustrates VeroE6 cells that were immobilized on beads, grown in suspension under gentle agitation, and infected with SARS-CoV-2 at the indicated MOI. 50% of the cells and media were harvested and replaced every other day. FIG. 6B shows flow cytometry plots of harvested cells stained for Propidium Iodide, a cell death marker. FIG. 6C graphically illustrates the percentage cell viability following SARS-CoV-2 infection at a MOI of 0.5. FIG. 6D graphically illustrates the cell viability (%) following SARS-CoV-2 infection at a MOI of 5.0. As shown in FIG. 6C-6D, the percentage of viable free cells (circular symbols) and viable immobilized cells (triangular symbols) exhibit an initial dip in cell viability, but the cultures recover by day 14 post infection.

FIGS. 7A-7B schematically illustrate the structures of two therapeutic interfering particles constructs for SARS-CoV-2, TIP1 and TIP2. FIG. 7A shows an example of the TIP1 construct structure. FIG. 7B shows an example of the TIP2 construct structure. The schematics show that TIP1 and TIP2 encode portions of the 5′ and 3′ untranslated regions (UTRs) of SARS-CoV-2. TIP1 encodes 450 nt of 5′UTR and 330 nt of 3′UTR. TIP2 includes the 5′UTR region and a larger portion of SARS-CoV-2 ORF1a (i.e., TIP2 encodes a deletion of ORF1a). Hence, TIP1 and TIP2 include the packaging signal but cannot express a functional copy of the viral ORF1a gene. The 3′UTR that is encoded by the TIP2 extends upstream 413 nt into the SARS-COV-2 N gene but TIP2 does not encode a functional form of the N gene (i.e., it encodes a deletion of part of the N gene). To facilitate analysis, the cassettes also include an IRES-mCherry reporter for flow cytometry analysis.

FIGS. 8A-8C graphically illustrate that four different types of therapeutic interfering particles (TIPs) reduce SARS-CoV-2 replication by more than 50-fold. FIG. 8A graphically illustrates the fold change in with SARS-CoV-2 RNA when various therapeutic interfering particles (TIPs) are present. Cells were transfected with mRNA of TIP1 (T1), TIP1* (T1*), TIP2 (T2) or TIP2* (T2*) and the cells were infected with SARS-CoV-2 (M01=0.005). Yield-reduction of SARS-CoV-2 replication was assessed by measuring the fold-reduction in SARS-CoV-2 mRNA (E gene) at 48 hrs post infection. mRNA was quantified by RT-qPCR with primers specific to 5′-end of N gene and the E gene that are not present in TIPs. The fold-reduction in SARS-CoV-2 mRNA as detected by E gene primers is shown. ITP2 exhibits the greatest interference with SARS-CoV-2. FIG. 8B graphically illustrates the relative LoglO amounts of SARS-CoV-2 genome when TIP1 and TIP2 therapeutic interfering particles are incubated for about 24 hours with the SARS-CoV-2 genome, as compared to control without the therapeutic interfering particles. FIG. 8C graphically illustrates the relative LoglO amounts of SARS-CoV-2 genome when TIP1 and TIP2 therapeutic interfering particles are incubated for about 48 hours with the SARS-CoV-2 genome, as compared to control without the therapeutic interfering particles.

FIGS. 9A-9B illustrate that TIP candidates are mobilized by SARS-CoV-2 and transmit together with SARS-CoV-2. FIG. 9A shows flow cytometry analysis of mCherry expression by Vero cells that received supernatant transferred from SARS-CoV-2 infected cells incubated with TIP1 and TIP2 therapeutic interfering particles compared to control cells receiving supernatant from naive uninfected cells that were incubated with the TIP1 and TIP2 particles. As shown, mCherry-expressing cells were detected when the TIP1 or TIP2 particles were present but essentially no mCherry-expressing cells were detected in the control cells. FIG. 9B graphically illustrates the log 10 amount of SARS-CoV-2 genome when TIP1 and TIP2 therapeutic interfering particles were incubated with cells that were infected with SARS-CoV-2 for 24 hours compared to controls that were not infected by SARS-CoV-2. FIG. 9C graphically illustrates the loglO amount of SARS-CoV-2 genome when TIP1 and TIP2 therapeutic interfering particles were incubated with cells that were infected with SARS-CoV-2 for 48 hours compared to controls that were not infected by SARS-CoV-2.

FIG. 10 schematically illustrates a method for interfering with SARS-CoV-2 transcription by transfection with antisense Transcription Regulating Sequences (TRS).

FIGS. 11A-11C graphically illustrate that antisense Transcription Regulating Sequences (TRS) can reduce SARS-CoV-2 plaque forming units (pfus). FIG. 11 A graphically illustrates the SARS-CoV-2 pfu after transfection with antisense TRS1 (ACGAACCUAAACACGAACCUAAAC (SEQ ID NO: 25)). FIG. 11B graphically illustrates the SARS-CoV-2 pfu after transfection with antisense TRS2 (ACGAACACGAACACGAACACGAAC (SEQ ID NO: 26)). FIG. 11C graphically illustrates the SARS-CoV-2 pfu after transfection with antisense TRS3 (CUAAACCUAAACCUAAACCUAAAC (SEQ ID NO: 27)).

FIG. 12 graphically illustrates that the combination of the TRS with either the TIP1 or the TIP2 significantly reduced the SARS-CoV-2 genome numbers compared to the TRS alone.

FIGS. 13A-13C illustrate that TIP1 and TIP2 therapeutic interfering particles significantly reduce the replication of different SARS-CoV-2 strains, including South African and U.K. strains of SARS-CoV-2. FIG. 13A illustrates that TIP1 and TIP2 significantly reduce the replication of South African 501Y.V2.HV delta variant of SARS-CoV-2. FIG. 13B illustrates that TIP1 and TIP2 significantly reduce the replication of South African 501Y.V2.HV variant of SARS-CoV-2. FIG. 13C illustrates that TIP1 and TIP2 significantly reduce the replication of U.K B.1.1.7 variant of SARS-CoV-2.

FIGS. 14A-14D show that SARS-CoV-2 TIPs inhibit SARS-CoV-2 in donor-derived lung organoids. FIG. 14A illustrates a schematic of primary human small-airway epithelial cell organoids. FIG. 14B shows an exemplary bright-field micrograph of organoids at day 2 following establishment from one representative donor (scale bar, 150 μm). FIG. 14C shows viral transcripts in SARS-CoV-2-infected (MOI=0.5) lung organoids transfected with control (Ctrl), TIP1, or TIP2 RNA assayed by qRT-PCR to envelope E gene at 24 h post-infection. FIG. 14D shows a viral titer quantification by plaque assay (PFU/mL) for samples shown in FIG. 14C. ***p<0.001, **p<0.01, *p<0.05 from Student's t test.

FIGS. 15A-15E show that SARS-CoV-2 TIP RNAs form functional VLPs, bind SARS-CoV-2 RdRp and nucleocapsid (N) trans elements, and mobilize with R0>1. FIG. 15A shows a reconstitution assay: schematic and quantification of VLP reconstitution for TIP1 and Ctrl RNA; Quantification in target cells by qRT-PCR for mCherry as compared to empty (RNA-free) VLPs. FIG. 15B shows an electromobility shift assay (EMSA) of TIP RNA or Ctrl RNA incubated with increasing concentrations of N protein or RdRp complex from cell extracts. FIG. 15C shows an R0 estimation via 1st-round supernatant transfer. TIP-transfected cells were infected with SARS-CoV-2 (MOI=0.05) and then thoroughly washed to remove virus, and at 2 h post-infection GFP+ reporter cells were introduced to the culture. At 12 h post-infection, GFP+ cells were analyzed by flow cytometry to quantify the percentage mCherry+ cells (via indirect immunofluorescence staining) within the GFP+ population. Uninfected cells were used as an experimental control to confirm that TIP mobilization only occurred in the presence of SARS-CoV-2. FIG. 15D shows a flow-cytometry quantification of FIG. 15C. FIG. 15E shows relative packaging of TIP RNA in virions. Cells were nucleofected with TIP1 or TIP2 followed by SARS-CoV-2 infection (MOI=0.05), and the supernatant was harvested at 24 h post-infection and analyzed by qRT-PCR for TIP RNA (using mCherry qPCR primers) versus viral genomic RNA (using E gene qPCR primers). Standard curves (see FIG. S3F) were statistically indistinguishable for both primer sets. ns, not significant, ****p<0.0001, **p<0.01, *p<0.05 from Student's t test.

FIGS. 16A-16D show that SARS-CoV-2 TIPs have a high barrier to the evolution of resistance in long-term cultures. FIG. 16A shows a schematic of the continual culture serial-passage system for SARS-CoV-2 propagation. Cells were transfected with TIP or Ctrl RNA and infected 24 h later with SARS-CoV-2 WA-1 isolate (at MOI=0.05). The cell-free supernatant was collected every 2 days for titering and transferred to naive cells. FIG. 16B shows viral titers of SARS-CoV-2 WA-1 by plaque assay (PFU/mL) from continuous cultures. Error bars represent three biological replicates. FIG. 16C shows a yield-reduction assay of virus isolated from day 24 of continuous culture tested in naive cells transfected with TIP RNA or Ctrl RNA. FIG. 16D shows the quantification of TIP and SARS-CoV-2 from day 20 of the continuous culture. Supernatants from day 20 of the continuous culture were analyzed by qRT-PCR for mCherry and E gene (i.e., SARS-CoV-2 genome) and the mCherry:E ratio was calculated: **p<0.01, *p<0.05 from Student's t test).

FIG. 17A shows bioluminescence imaging of mice six hours after intranasal administration of in vitro transcribed RNA encoding firefly luciferase. Mice were given either saline, purified RNA alone (‘naked RNA’), or LNP-encapsulated RNA.

FIG. 17B shows dynamic light scattering (DLS) characterization of LNPs carrying TIP RNA to measure radius and polydispersity (left panel), and validation of antiviral activity (yield reduction) of LNP TIPs in infected Vero cells by plaque assay (PFU/ml) (right panel).

FIG. 17C shows a timeline of SARS-CoV-2 challenge experiment in Syrian golden hamsters. At 6 h pre-infection, intranasally administration of TIP LNPs (n=5) or Ctrl RNA LNPs (n=5) was performed. Animals were then infected with SARS-CoV-2 (106 PFU), and an intranasal LNP booster was administration delivered at 18 h post-infection. Lungs were harvested at 5 days post-infection.

FIG. 17D shows the weight change of hamsters over time after infection with SARS-CoV-2 in Ctrl- or TIP LNP treated animals following the SARS-CoV-2 challenge protocol as outlined in FIG. 17C.

FIG. 17E shows the SARS-CoV-2 viral titers from lungs harvested on day 5, following the SARS-CoV-2 challenge protocol as outlined in FIG. 17C, by plaque assay. ***p<0.001, **p <0.01, *p<0.05 from Student's t test.

FIG. 17F shows SARS-CoV-2 viral transcript levels by qRT-PCR for N, NSP14, and E from lungs harvested on day 5 post-infection following the SARS-CoV-2 challenge protocol as outlined in FIG. 17C. ***p<0.001, **p<0.01, *p<0.05 from Student's t test.

FIG. 18A shows mCherry RNA levels in lungs of TIP and Ctrl RNA-treated animals on day 5, following the SARS-CoV-2 challenge protocol as outlined in FIG. 17C, by qRT-PCR

FIG. 18B shows luciferase RNA levels from lungs harvested from TIP and Ctrl RNA-treated animals on day 5, following the SARS-CoV-2 challenge protocol as outlined in FIG. 17C, by qRT-PCR. ***p<0.001, **p<0.01, *p<0.05 from Student's t test.

FIG. 18C shows quantification of TIP and Ctrl RNA in the presence and absence of infection in hamsters. Syrian golden hamsters were treated twice with TIP or Ctrl RNA at 24 hrs apart in the presence and absence of SARS-CoV-2 (106 PFU). Lungs were harvested at day 5, RNA was extracted, and qRT-PCR was performed for either mCherry or luciferase. Quantification of TIP and Ctrl RNAs was performed between the infected and uninfected lung samples. ***p<0.001, **p<0.01, *p<0.05 from Student's t test.

FIG. 19A shows differential gene expression (differentially expressed genes, DEGs) in hamster lungs on day 5 post infection by RNaseq analysis. Each column represents one animal clustered by expression profiles. DEGs were defined by comparing infected samples treated with TIP RNA or Ctrl RNA LNPs, and are grouped in four clusters.

FIG. 19B shows a Venn diagram of RNA sequencing of hamster lungs summarizing (DEGs) in TIP versus Ctrl-treated animals using the Interferome database with parameters Mus musculus to approximate Syrian golden hamster. The majority of the DEGs in cluster III are interferon-stimulated genes (ISGs), regulated by either Type I or Type II interferons (IFNs).

FIG. 19C shows a gene ontology (GO) analysis showing the top ten biological processes enriched in cluster III.

FIG. 19D shows differential gene expression in lungs on day 5 by RNA-seq analysis. Each column represents one animal clustered by expression profiles and uninfected hamster data obtained from GSE157058. Cluster III genes are shown in the heatmap.

FIG. 19E shows expression levels for a subset of pro-inflammatory cytokines and IFN-response genes. *** denotes p<0.001, ** denotes p<0.01, * denotes p<0.05 from Student's t test.

FIG. 19F shows expression levels in terms of transcripts per million (TPM) for representative genes belonging to cytokine/chemokine pathways (individual animals are shown as individual data points). These proinflammatory cytokines (Ccl7, Ccr1, Cxcl10, Cxcl11) were previously reported to be upregulated in COVID-19 patients, but are significantly reduced in TIP-treated animals. * denotes p<0.05 from Student's t test.

FIG. 19G shows a heatmap showing expression level of DEGs in uninfected samples. DEGs were defined by comparing infected samples treated with TIP or Ctrl RNA LNPs. Representative proinflammatory genes are shown on the right in the presence and absence of infection. ns denotes not significant, **** denotes p<0.0001 *** denotes p<0.001, ** denotes p<0.01, * denotes p<0.05 from Student's t test.

FIG. 20A shows H&E staining of lung section of one representative Ctrl- and TIP-administered animal. Asterisks indicate alveolar edemas, and at signs indicate cellular infiltrates to alveolar space.

FIG. 20B shows histopathology imaging of Syrian hamster lungs following pre-infection treatment. Micrographs of brightfield imaging of H&E-stained lung sections from all animals (top: Ctrl RNA treated hamsters; bottom: TIP-treated hamsters). Stitched images were analyzed using Leica Aperio ImageScope software. For each animal, whole lung shown on above and a representative zoomed-in section to visualize histopathology shown below. Scale bars are as indicated, n=5 for each group. Labels that were added to the raw images during sectioning were covered during figure preparation, and size bars were added to the image.

FIG. 20C shows histopathological scoring of lung sections for alveolar edema (left) and cellular infiltrates to alveolar space (right). ** denotes p<0.01 from Student's t test.

FIG. 20D shows H&E staining of a lung section of one representative Ctrl-treated and one representative TIP-treated animal in the absence of infection at 5 days post treatment (left). Histopathological scoring of lung sections from the uninfected hamsters (n=3 for each group of animals) treated with TIP or Ctrl RNA LNPs for alveolar edema and cellular infiltrates to alveolar space (right).

FIG. 21A shows a schematic of post-infection treatment experiment. Animals were infected with SARS-CoV-2 (106 PFU) and, at 12 h post-infection, a single-administration of TIP or Ctrl RNA LNPs (n=5 each) was intranasally administered.

FIG. 21B shows SARS-CoV-2 viral titers in lungs on day 5 of post-infection treatment experiment (outlined in FIG. 21A) by plaque assay. ** denotes p<0.01 from Student's t test.

FIG. 21C shows H&E staining of lung section of one representative post-infection Ctrl- and TIP-treated animal. The asterisks indicate alveolar edemas, and the at signs indicate cellular infiltrates to alveolar space. *p<0.01, obtained from a permutation test.

FIG. 21D shows histopathological scoring of lung sections for cellular infiltrates to alveolar space. *p<0.01, obtained from a permutation test.

FIG. 21E shows histopathology imaging of Syrian hamster lungs following post-infection treatment. Micrographs of brightfield imaging of H&E-stained lung sections from all animals (top: Ctrl RNA treated hamsters; bottom: TIP-treated hamsters). Stitched images were analyzed using Leica Aperio ImageScope software. For each animal, whole lung shown on above and a representative zoomed-in section to visualize histopathology shown below. Scale bars are as indicated, n=5 for each group. Labels that were added to the raw images during sectioning were covered during figure preparation, and size bars were added to the image.

### DETAILED DESCRIPTION

Described herein are compositions of robust, therapeutic SARS-CoV-2 DIPs (i.e., Therapeutic Interfering Particles, TIPs), such as a recombinant SARS-CoV-2 construct capable of interfering with SARS-CoV-2 replication. The recombinant SARS-CoV-2 construct cannot replicate by itself, but can replicate in the presence of infective SARS-CoV-2 (e.g., replication competent SARS-CoV-2). The TIPs are shown to conditionally replicate with SARS-Cov-2, exhibiting basic reproductive ratio (R₀)>1, and inhibit viral replication 10- to 100-fold. Inhibition occurs via competition for viral replication machinery, and a single administration of TIP RNA was shown to inhibit SARS-CoV-2 sustainably in continuous cultures. It was demonstrated strikingly that TIPs maintain efficacy against neutralization-resistant variants (e.g., B.1.351). In hamster models of SARS-CoV-2 infection, both prophylactic and therapeutic intranasal administration of TIPs in lipid nanoparticles durably suppressed SARS-CoV-2 by 100-fold in the lungs, reduced pro-inflammatory cytokine expression, and prevented severe pulmonary edema. These data demonstrate successful therapeutic and prophylactic use of TIPs described herein against SARS-CoV-2 infection.

Thus, the present application in one aspect provides a recombinant SARS-CoV-2 construct (e.g., SARS-CoV-2 TIP) capable of interfering with SARS-CoV-2 replication, wherein the recombinant SARS-CoV-2 construct cannot replicate by itself, and wherein the recombinant SARS-CoV-2 construct can replicate in the presence of SARS-CoV-2. Further provided are delivery vehicles, such as lipid nanoparticles, comprising such recombinant SARS-CoV-2 constructs (e.g., SARS-CoV-2 TIPs) Also provided are viral-like particles or a cell comprising such recombinant SARS-CoV-2 construct.

In another aspect, there are provided a pharmaceutical composition comprising a recombinant SARS-CoV-2 construct (e.g., SARS-CoV-2 TIP) capable of interfering with SARS-CoV-2 replication, wherein the recombinant SARS-CoV-2 construct cannot replicate by itself, and wherein the recombinant SARS-CoV-2 construct can replicate in the presence of SARS-CoV-2, as well as uses thereof for treating and/or preventing SARS-CoV-2.

### I. Definitions

A “wild-type strain of a virus” is a strain that does not comprise any of the human made mutations as described herein, i.e., a wild-type virus is any virus that can be isolated from nature (e.g., from a human infected with the virus). A wild-type virus can be cultured in a laboratory, but still, in the absence of any other virus, is capable of producing progeny genomes or virions like those isolated from nature.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it: (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of an agent (e.g., a construct, a particle, etc., as described herein) that, when administered to a mammal (e.g., a human) or other subject for treating a disease, is sufficient to effect such treatment for the disease. The “therapeutically effective amount” can vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject such as a mammal, e.g., a human. In general a “pharmaceutical composition” is sterile and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intrabracheal and the like.

All numerical designations, for example, temperature, time, concentration, viral load, and molecular weight, including ranges, are approximations which are varied (+) or (−) by increments of 0.1 or 1.0, where appropriate. rt is to he understood, although not always explicitly stated that all numerical designations arc preceded by the term “about”

It also is to be understood, although not always explicitly stated, that the reagents described herein are merely exemplary and that in some cases equivalents may be available in the art.

Also, as used herein, “and/or” refers to and encompasses any and all possible combinations of one or more of the associated listed items, as well as the lack of combinations when interpreted in the alternative (“or”).

It must be noted that as used herein and in the appended claims, the singular forms “a,” “an,” and “the” include plural referents unless the context dearly dictates otherwise. Thus, for example, reference to “an interfering particle” includes a plurality of such particles and reference to “the cis-acting element” includes reference to one or more cis-acting elements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications me cited.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

The following statements provide a summary of some aspects of the inventive nucleic acids and methods described herein.

### II. Recombinant SARS-CoV-2 Constructs

The present application provides recombinant SARS-CoV-2 constructs capable of interfering with SARS-CoV-2 replication, and delivery vehicles such as lipid nanoparticles comprising such constructs (also referred to herein as SARS-CoV-2 therapeutic interfering particles (TIPs), TIP constructs). In some embodiments, the recombinant SARS-CoV-2 constructs and SARS-CoV-2 TIPs can reduce SARS-CoV-2 replication by more than any of 10, 20, 30, 40, 50, 60, 70, 80, 90, or 100-fold. The recombinant SARS-CoV-2 constructs and SARS-CoV-2 TIPs can include segments of the 5′ and 3′ ends of the SARS-CoV-2 genome. For example, the recombinant SARS-CoV-2 constructs and SARS-CoV-2 TIPs can comprise segments of the 5′-UTR and the 3′-UTR of SARS-CoV-2. An intervening sequence (e.g., a SARS-CoV-2 sequence and/or a heterologous sequence, such as a detectable marker protein and/or a unique molecular identifier (UMI) sequence) may be placed between the 5′ and 3′ segments of the SARS-CoV-2 genome. The recombinant SARS-CoV-2 construct cannot replicate by itself, but is able to replicate in the presence of SARS-CoV-2. In some embodiments, the recombinant SARS-CoV-2 construct is a DNA, RNA, mRNA, or a combination thereof.

Provided herein is a recombinant SARS-CoV-2 construct capable of interfering with SARS-CoV-2 replication (e.g., SARS-CoV-2 TIP), comprising: (a) a 5′UTR region comprising at least 100 nucleotides of a SARS-Cov-2 5′UTR or a variant thereof, (b) an optional intervening sequence, and (c) a 3′UTR region comprising at least 100 nucleotides of a SARS-Cov-2 3′UTR or a variant thereof, wherein the recombinant SARS-CoV-2 construct cannot replicate by itself, wherein the recombinant SARS-CoV-2 construct can replicate in the presence of SARS-CoV-2. In some embodiments, the intervening sequence has a length of about 1 base pairs (bp) to about 29000 bp, including for example about any of 1-25000, 1-20000, 1-15000, 1-10000, 1-900, 1-800, 1-700, 1-600, 1-500, 1-400, 1-300, 1-200, or 1-100 bp. In some embodiments, the intervening sequence comprises a SARS-CoV-2 sequence, a heterologous sequence, or a combination thereof. In some embodiments, the SARS-CoV-2 sequence does not encode a functional viral protein. In some embodiments, the recombinant SARS-CoV-2 construct comprises a packaging signal for SAR-CoV-2. In some embodiments, the packaging signal comprises stem loop 5 in the SARS-CoV-2 5′UTR. In some embodiments, the recombinant SARS-CoV-2 construct comprises a 3′ modification or 3′ extended sequence (such as a polyA sequence or a signaling sequence or polyA addition). In some embodiments, the recombinant SARS-CoV-2 construct comprises a 5′ modification (such as a 5′ methyl cap). In some embodiments, the recombinant SARS-CoV-2 construct genomic RNA is produced at a higher rate than SARS-CoV-2 genomic RNA when present in a host cell infected with SARS-CoV-2, such that the ratio of the construct SAR-CoV-2 genomic RNA to the SARS-CoV-2 genomic RNA is greater than 1 in the cell. In some embodiments, the recombinant SARS-CoV-2 construct has a same or lower transmission frequency than SARS-CoV-2. In some embodiments, the recombinant SARS-CoV-2 construct has a higher transmission frequency than SARS-CoV-2. In some embodiments, the recombinant SARS-CoV-2 construct is packaged with the same or a higher efficiency than SARS-CoV-2 when present in a host cell infected with SARS-CoV-2. In some embodiments, the recombinant SARS-CoV-2 construct has a basic reproduction ratio (R₀) >1.

In some embodiments, provided herein is a recombinant SARS-CoV-2 construct capable of interfering with SARS-CoV-2 replication (e.g., SARS-CoV-2 TIP), comprising: (a) a 5′UTR region comprising nucleotides 1-265 of SEQ ID NO: 1 or a variant thereof, (b) an intervening sequence, and (c) a 3′UTR region comprising nucleotides 29675-29870 or nucleotides 29675-29903 of SEQ ID NO: 1 or a variant thereof, wherein the recombinant SARS-CoV-2 construct cannot replicate by itself, wherein the recombinant SARS-CoV-2 construct can replicate in the presence of infective SARS-CoV-2, and wherein the intervening sequence is about 1 base pairs (bp) to about 29000 bp. In some embodiments, the total length of the 5′UTR region, the optional intervening sequence, and the 3′UTR region in the recombinant SARS-CoV-2 construct is about 2000 bp to about 3500 bp, such as about 2100 bp. In some embodiments, the intervening sequence comprises a SARS-CoV-2 sequence, a heterologous sequence, or a combination thereof. In some embodiments, the SARS-CoV-2 sequence does not encode a functional viral protein. In some embodiments, the recombinant SARS-CoV-2 construct comprises a packaging signal for SAR-CoV-2. In some embodiments, the packaging signal comprises stem loop 5 in the SARS-CoV-2 5′UTR. In some embodiments, the recombinant SARS-CoV-2 construct comprises a 3′ modification or 3′ extended sequence (such as a polyA sequence or a signaling sequence or polyA addition). In some embodiments, the recombinant SARS-CoV-2 construct comprises a 5′ modification (such as a 5′ methyl cap).

In some embodiments, provided herein is a recombinant SARS-CoV-2 construct capable of interfering with SARS-CoV-2 replication (e.g., SARS-CoV-2 TIP), comprising: (i) nucleotides 1-450 of SEQ ID NO: 1 or a variant thereof, and (ii) nucleotides 29543-29870 or nucleotides 29543-29903 of SEQ ID NO: 1 or a variant thereof, wherein the recombinant SARS-CoV-2 construct cannot replicate by itself, wherein the recombinant SARS-CoV-2 construct can replicate in the presence of infective SARS-CoV-2. In some embodiments, the recombinant SARS-CoV-2 construct comprises a cytosine (C) to thymine (T) mutation at nucleotide 241 of SEQ ID NO: 1. In some embodiments, the total length of the 5′UTR region, the optional intervening sequence, and the 3′UTR region in the recombinant SARS-CoV-2 construct is about 2000 bp to about 3500 bp, such as about 2100 bp. In some embodiments, the recombinant SARS-CoV-2 construct comprises a packaging signal for SAR-CoV-2. In some embodiments, the packaging signal comprises stem loop 5 in the SARS-CoV-2 5′UTR. In some embodiments, the recombinant SARS-CoV-2 construct comprises a 3′ modification or 3′ extended sequence (such as a polyA sequence or a signaling sequence or polyA addition). In some embodiments, the recombinant SARS-CoV-2 construct comprises a 5′ modification (such as a 5′ methyl cap). In some embodiments, the recombinant SARS-CoV-2 construct comprises a 5′UTR comprising the amino acid sequence of SEQ ID NO: 28, and a 3′UTR comprising the amino acid sequence of SEQ ID NO: 29. In some embodiments, the recombinant SARS-CoV-2 construct comprises a 5′UTR comprising the amino acid sequence of SEQ ID NO: 32, and a 3′UTR comprising the amino acid sequence of SEQ ID NO: 29.

In some embodiments, provided herein is a recombinant SARS-CoV-2 construct capable of interfering with SARS-CoV-2 replication (e.g., SARS-CoV-2 TIP), comprising: (i) nucleotides 1-1540 of SEQ ID NO: 1 or a variant thereof, and (ii) nucleotides 29191-29870 or nucleotides 29191-29903 of SEQ ID NO: 1 or a variant thereof, wherein the recombinant SARS-CoV-2 construct cannot replicate by itself, wherein the recombinant SARS-CoV-2 construct can replicate in the presence of infective SARS-CoV-2. In some embodiments, the recombinant SARS-CoV-2 construct comprises a C to a T mutation at nucleotide 241 of SEQ ID NO: 1. In some embodiments, the total length of the 5′UTR region, the optional intervening sequence, and the 3′UTR region in the recombinant SARS-CoV-2 construct is about 2000 bp to about 3500 bp, such as about 3500 bp. In some embodiments, the recombinant SARS-CoV-2 construct comprises a packaging signal for SAR-CoV-2. In some embodiments, the packaging signal comprises stem loop 5 in the SARS-CoV-2 5′UTR. In some embodiments, the recombinant SARS-CoV-2 construct comprises a 3′ modification or 3′ extended sequence (such as a polyA sequence or a signaling sequence or polyA addition). In some embodiments, the recombinant SARS-CoV-2 construct comprises a 5′ modification (such as a 5′ methyl cap). In some embodiments, the recombinant SARS-CoV-2 construct comprises a 5′UTR comprising the amino acid sequence of SEQ ID NO: 30, and a 3′UTR comprising the amino acid sequence of SEQ ID NO: 31. In some embodiments, the recombinant SARS-CoV-2 construct comprises a 5′UTR comprising the amino acid sequence of SEQ ID NO: 33, and a 3′UTR comprising the amino acid sequence of SEQ ID NO: 31.

### A. SARS-CoV-2

In some embodiments, a recombinant SARS-CoV-2 construct comprises a nucleic acid sequence of a SARS-CoV-2 virus, a fragment of a nucleic acid sequence a SARS-CoV-2 virus, a nucleic acid sequence of a variant of a SARS-CoV-2 virus, or a fragment of a nucleic acid sequence of a variant of a SARS-CoV-2 virus.

The SARS-CoV-2 virus has a single-stranded RNA genome with about 29891 nucleotides that encode about 9860 amino acids. A SARS-CoV-2 selected RNA genome can be copied and made into a DNA by reverse transcription and formation of a cDNA. A linear SARS-CoV-2 DNA can be circularized by ligation of SARS-CoV-2 DNA ends.

As used herein, a “SARS-CoV-2 genome” refers to the 29903 nucleotide sequence described by NIH GenBank Locus NC_045512, or the hCoV-19 reference sequence described by the Global Initiative on Sharing Avian Influenza Data (GISAID).

A DNA sequence for the SARS-CoV-2 genome, with coding regions, is available as accession number NC_045512.2 from the NCBI website (provided as SEQ ID NO: 1 herein). In some embodiments, the recombinant SARS-CoV-2 construct comprises SEQ ID NO: 1, or a sequence comprising at least about 90% sequence identity (such as at least any of about 91%, 92%, 93%, 94%, 95%, 96%, 97%, 98%, or 99% sequence identity) to the sequence of SEQ ID NO: 1.

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | CGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 441 | GCGTTTTGCC TCAACTTGAA CAGCCCTATG TGTTCATCAA |
| 481 | ACGTTCGGAT GCTCGAACTG CACCTCATGG TCATGTTATG |
| 521 | GTTGAGCTGG TAGCAGAACT CGAAGGCATT CAGTACGGTC |
| 561 | GTAGTGGTGA GACACTTGGT GTCCTTGTCC CTCATGTGGG |
| 601 | CGAAATACCA GTGGCTTACC GCAAGGTTCT TCTTCGTAAG |
| 641 | AACGGTAATA AAGGAGCTGG TGGCCATAGT TACGGCGCCG |
| 681 | ATCTAAAGTC ATTTGACTTA GGCGACGAGC TTGGCACTGA |
| 721 | TCCTTATGAA GATTTTCAAG AAAACTGGAA CACTAAACAT |
| 761 | AGCAGTGGTG TTACCCGTGA ACTCATGCGT GAGCTTAACG |
| 801 | GAGGGGCATA CACTCGCTAT GTCGATAACA ACTTCTGTGG |
| 841 | CCCTGATGGC TACCCTCTTG AGTGCATTAA AGACCTTCTA |
| 881 | GCACGTGCTG GTAAAGCTTC ATGCACTTTG TCCGAACAAC |
| 921 | TGGACTTTAT TGACACTAAG AGGGGTGTAT ACTGCTGCCG |
| 961 | TGAACATGAG CATGAAATTG CTTGGTACAC GGAACGTTCT |
| 1001 | GAAAAGAGCT ATGAATTGCA GACACCTTTT GAAATTAAAT |
| 1041 | TGGCAAAGAA ATTTGACACC TTCAATGGGG AATGTCCAAA |
| 1081 | TTTTGTATTT CCCTTAAATT CCATAATCAA GACTATTCAA |
| 1121 | CCAAGGGTTG AAAAGAAAAA GCTTGATGGC TTTATGGGTA |
| 1161 | GAATTCGATC TGTCTATCCA GTTGCGTCAC CAAATGAATG |
| 1201 | CAACCAAATG TGCCTTTCAA CTCTCATGAA GTGTGATCAT |
| 1241 | TGTGGTGAAA CTTCATGGCA GACGGGCGAT TTTGTTAAAG |
| 1281 | CCACTTGCGA ATTTTGTGGC ACTGAGAATT TGACTAAAGA |
| 1321 | AGGTGCCACT ACTTGTGGTT ACTTACCCCA AAATGCTGTT |
| 1361 | GTTAAAATTT ATTGTCCAGC ATGTCACAAT TCAGAAGTAG |
| 1401 | GACCTGAGCA TAGTCTTGCC GAATACCATA ATGAATCTGG |
| 1441 | CTTGAAAACC ATTCTTCGTA AGGGTGGTCG CACTATTGCC |
| 1481 | TTTGGAGGCT GTGTGTTCTC TTATGTTGGT TGCCATAACA |
| 1521 | AGTGTGCCTA TTGGGTTCCA CGTGCTAGCG CTAACATAGG |
| 1561 | TTGTAACCAT ACAGGTGTTG TTGGAGAAGG TTCCGAAGGT |
| 1601 | CTTAATGACA ACCTTCTTGA AATACTCCAA AAAGAGAAAG |
| 1641 | TCAACATCAA TATTGTTGGT GACTTTAAAC TTAATGAAGA |
| 1681 | GATCGCCATT ATTTTGGCAT CTTTTTCTGC TTCCACAAGT |
| 1721 | GCTTTTGTGG AAACTGTGAA AGGTTTGGAT TATAAAGCAT |
| 1761 | TCAAACAAAT TGTTGAATCC TGTGGTAATT TTAAAGTTAC |
| 1801 | AAAAGGAAAA GCTAAAAAAG GTGCCTGGAA TATTGGTGAA |
| 1841 | CAGAAATCAA TACTGAGTCC TCTTTATGCA TTTGCATCAG |
| 1881 | AGGCTGCTCG TGTTGTACGA TCAATTTTCT CCCGCACTCT |
| 1921 | TGAAACTGCT CAAAATTCTG TGCGTGTTTT ACAGAAGGCC |
| 1961 | GCTATAACAA TACTAGATGG AATTTCACAG TATTCACTGA |
| 2001 | GACTCATTGA TGCTATGATG TTCACATCTG ATTTGGCTAC |
| 2041 | TAACAATCTA GTTGTAATGG CCTACATTAC AGGTGGTGTT |
| 2081 | GTTCAGTTGA CTTCGCAGTG GCTAACTAAC ATCTTTGGCA |
| 2121 | CTGTTTATGA AAAACTCAAA CCCGTCCTTG ATTGGCTTGA |
| 2161 | AGAGAAGTTT AAGGAAGGTG TAGAGTTTCT TAGAGACGGT |
| 2201 | TGGGAAATTG TTAAATTTAT CTCAACCTGT GCTTGTGAAA |
| 2241 | TTGTCGGTGG ACAAATTGTC ACCTGTGCAA AGGAAATTAA |
| 2281 | GGAGAGTGTT CAGACATTCT TTAAGCTTGT AAATAAATTT |
| 2321 | TTGGCTTTGT GTGCTGACTC TATCATTATT GGTGGAGCTA |
| 2361 | AACTTAAAGC CTTGAATTTA GGTGAAACAT TTGTCACGCA |
| 2401 | CTCAAAGGGA TTGTACAGAA AGTGTGTTAA ATCCAGAGAA |
| 2441 | GAAACTGGCC TACTCATGCC TCTAAAAGCC CCAAAAGAAA |
| 2481 | TTATCTTCTT AGAGGGAGAA ACACTTCCCA CAGAAGTGTT |
| 2521 | AACAGAGGAA GTTGTCTTGA AAACTGGTGA TTTACAACCA |
| 2561 | TTAGAACAAC CTACTAGTGA AGCTGTTGAA GCTCCATTGG |
| 2601 | TTGGTACACC AGTTTGTATT AACGGGCTTA TGTTGCTCGA |
| 2641 | AATCAAAGAC ACAGAAAAGT ACTGTGCCCT TGCACCTAAT |
| 2681 | ATGATGGTAA CAAACAATAC CTTCACACTC AAAGGCGGTG |
| 2721 | CACCAACAAA GGTTACTTTT GGTGATGACA CTGTGATAGA |
| 2761 | AGTGCAAGGT TACAAGAGTG TGAATATCAC TTTTGAACTT |
| 2801 | GATGAAAGGA TTGATAAAGT ACTTAATGAG AAGTGCTCTG |
| 2841 | CCTATACAGT TGAACTCGGT ACAGAAGTAA ATGAGTTCGC |
| 2881 | CTGTGTTGTG GCAGATGCTG TCATAAAAAC TTTGCAACCA |
| 2921 | GTATCTGAAT TACTTACACC ACTGGGCATT GATTTAGATG |
| 2961 | AGTGGAGTAT GGCTACATAC TACTTATTTG ATGAGTCTGG |
| 3001 | TGAGTTTAAA TTGGCTTCAC ATATGTATTG TTCTTTCTAC |
| 3041 | CCTCCAGATG AGGATGAAGA AGAAGGTGAT TGTGAAGAAG |
| 3081 | AAGAGTTTGA GCCATCAACT CAATATGAGT ATGGTACTGA |
| 3121 | AGATGATTAC CAAGGTAAAC CTTTGGAATT TGGTGCCACT |
| 3161 | TCTGCTGCTC TTCAACCTGA AGAAGAGCAA GAAGAAGATT |
| 3201 | GGTTAGATGA TGATAGTCAA CAAACTGTTG GTCAACAAGA |
| 3241 | CGGCAGTGAG GACAATCAGA CAACTACTAT TCAAACAATT |
| 3281 | GTTGAGGTTC AACCTCAATT AGAGATGGAA CTTACACCAG |
| 3321 | TTGTTCAGAC TATTGAAGTG AATAGTTTTA GTGGTTATTT |
| 3361 | AAAACTTACT GACAATGTAT ACATTAAAAA TGCAGACATT |
| 3401 | GTGGAAGAAG CTAAAAAGGT AAAACCAACA GTGGTTGTTA |
| 3441 | ATGCAGCCAA TGTTTACCTT AAACATGGAG GAGGTGTTGC |
| 3481 | AGGAGCCTTA AATAAGGCTA CTAACAATGC CATGCAAGTT |
| 3521 | GAATCTGATG ATTACATAGC TACTAATGGA CCACTTAAAG |
| 3561 | TGGGTGGTAG TTGTGTTTTA AGCGGACACA ATCTTGCTAA |
| 3601 | ACACTGTCTT CATGTTGTCG GCCCAAATGT TAACAAAGGT |
| 3641 | GAAGACATTC AACTTCTTAA GAGTGCTTAT GAAAATTTTA |
| 3681 | ATCAGCACGA AGTTCTACTT GCACCATTAT TATCAGCTGG |
| 3721 | TATTTTTGGT GCTGACCCTA TACATTCTTT AAGAGTTTGT |
| 3761 | GTAGATACTG TTCGCACAAA TGTCTACTTA GCTGTCTTTG |
| 3801 | ATAAAAATCT CTATGACAAA CTTGTTTCAA GCTTTTTGGA |
| 3841 | AATGAAGAGT GAAAAGCAAG TTGAACAAAA GATCGCTGAG |
| 3881 | ATTCCTAAAG AGGAAGTTAA GCCATTTATA ACTGAAAGTA |
| 3921 | AACCTTCAGT TGAACAGAGA AAACAAGATG ATAAGAAAAT |
| 3961 | CAAAGCTTGT GTTGAAGAAG TTACAACAAC TCTGGAAGAA |
| 4001 | ACTAAGTTCC TCACAGAAAA CTTGTTACTT TATATTGACA |
| 4041 | TTAATGGCAA TCTTCATCCA GATTCTGCCA CTCTTGTTAG |
| 4081 | TGACATTGAC ATCACTTTCT TAAAGAAAGA TGCTCCATAT |
| 4121 | ATAGTGGGTG ATGTTGTTCA AGAGGGTGTT TTAACTGCTG |
| 4161 | TGGTTATACC TACTAAAAAG GCTGGTGGCA CTACTGAAAT |
| 4201 | GCTAGCGAAA GCTTTGAGAA AAGTGCCAAC AGACAATTAT |
| 4241 | ATAACCACTT ACCCGGGTCA GGGTTTAAAT GGTTACACTG |
| 4281 | TAGAGGAGGC AAAGACAGTG CTTAAAAAGT GTAAAAGTGC |
| 4321 | CTTTTACATT CTACCATCTA TTATCTCTAA TGAGAAGCAA |
| 4361 | GAAATTCTTG GAACTGTTTC TTGGAATTTG CGAGAAATGC |
| 4401 | TTGCACATGC AGAAGAAACA CGCAAATTAA TGCCTGTCTG |
| 4441 | TGTGGAAACT AAAGCCATAG TTTCAACTAT ACAGCGTAAA |
| 4481 | TATAAGGGTA TTAAAATACA AGAGGGTGTG GTTGATTATG |
| 4521 | GTGCTAGATT TTACTTTTAC ACCAGTAAAA CAACTGTAGC |
| 4561 | GTCACTTATC AACACACTTA ACGATCTAAA TGAAACTCTT |
| 4601 | GTTACAATGC CACTTGGCTA TGTAACACAT GGCTTAAATT |
| 4641 | TGGAAGAAGC TGCTCGGTAT ATGAGATCTC TCAAAGTGCC |
| 4681 | AGCTACAGTT TCTGTTTCTT CACCTGATGC TGTTACAGCG |
| 4721 | TATAATGGTT ATCTTACTTC TTCTTCTAAA ACACCTGAAG |
| 4761 | AACATTTTAT TGAAACCATC TCACTTGCTG GTTCCTATAA |
| 4801 | AGATTGGTCC TATTCTGGAC AATCTACACA ACTAGGTATA |
| 4841 | GAATTTCTTA AGAGAGGTGA TAAAAGTGTA TATTACACTA |
| 4881 | GTAATCCTAC CACATTCCAC CTAGATGGTG AAGTTATCAC |
| 4921 | CTTTGACAAT CTTAAGACAC TTCTTTCTTT GAGAGAAGTG |
| 4961 | AGGACTATTA AGGTGTTTAC AACAGTAGAC AACATTAACC |
| 5001 | TCCACACGCA AGTTGTGGAC ATGTCAATGA CATATGGACA |
| 5041 | ACAGTTTGGT CCAACTTATT TGGATGGAGC TGATGTTACT |
| 5081 | AAAATAAAAC CTCATAATTC ACATGAAGGT AAAACATTTT |
| 5121 | ATGTTTTACC TAATGATGAC ACTCTACGTG TTGAGGCTTT |
| 5161 | TGAGTACTAC CACACAACTG ATCCTAGTTT TCTGGGTAGG |
| 5201 | TACATGTCAG CATTAAATCA CACTAAAAAG TGGAAATACC |
| 5241 | CACAAGTTAA TGGTTTAACT TCTATTAAAT GGGCAGATAA |
| 5281 | CAACTGTTAT CTTGCCACTG CATTGTTAAC ACTCCAACAA |
| 5321 | ATAGAGTTGA AGTTTAATCC ACCTGCTCTA CAAGATGCTT |
| 5361 | ATTACAGAGC AAGGGCTGGT GAAGCTGCTA ACTTTTGTGC |
| 5401 | ACTTATCTTA GCCTACTGTA ATAAGACAGT AGGTGAGTTA |
| 5441 | GGTGATGTTA GAGAAACAAT GAGTTACTTG TTTCAACATG |
| 5481 | CCAATTTAGA TTCTTGCAAA AGAGTCTTGA ACGTGGTGTG |
| 5521 | TAAAACTTGT GGACAACAGC AGACAACCCT TAAGGGTGTA |
| 5561 | GAAGCTGTTA TGTACATGGG CACACTTTCT TATGAACAAT |
| 5601 | TTAAGAAAGG TGTTCAGATA CCTTGTACGT GTGGTAAACA |
| 5641 | AGCTACAAAA TATCTAGTAC AACAGGAGTC ACCTTTTGTT |
| 5681 | ATGATGTCAG CACCACCTGC TCAGTATGAA CTTAAGCATG |
| 5721 | GTACATTTAC TTGTGCTAGT GAGTACACTG GTAATTACCA |
| 5761 | GTGTGGTCAC TATAAACATA TAACTTCTAA AGAAACTTTG |
| 5801 | TATTGCATAG ACGGTGCTTT ACTTACAAAG TCCTCAGAAT |
| 5841 | ACAAAGGTCC TATTACGGAT GTTTTCTACA AAGAAAACAG |
| 5881 | TTACACAACA ACCATAAAAC CAGTTACTTA TAAATTGGAT |
| 5921 | GGTGTTGTTT GTACAGAAAT TGACCCTAAG TTGGACAATT |
| 5961 | ATTATAAGAA AGACAATTCT TATTTCACAG AGCAACCAAT |
| 6001 | TGATCTTGTA CCAAACCAAC CATATCCAAA CGCAAGCTTC |
| 6041 | GATAATTTTA AGTTTGTATG TGATAATATC AAATTTGCTG |
| 6081 | ATGATTTAAA CCAGTTAACT GGTTATAAGA AACCTGCTTC |
| 6121 | AAGAGAGCTT AAAGTTACAT TTTTCCCTGA CTTAAATGGT |
| 6161 | GATGTGGTGG CTATTGATTA TAAACACTAC ACACCCTCTT |
| 6201 | TTAAGAAAGG AGCTAAATTG TTACATAAAC CTATTGTTTG |
| 6241 | GCATGTTAAC AATGCAACTA ATAAAGCCAC GTATAAACCA |
| 6281 | AATACCTGGT GTATACGTTG TCTTTGGAGC ACAAAACCAG |
| 6321 | TTGAAACATC AAATTCGTTT GATGTACTGA AGTCAGAGGA |
| 6361 | CGCGCAGGGA ATGGATAATC TTGCCTGCGA AGATCTAAAA |
| 6401 | CCAGTCTCTG AAGAAGTAGT GGAAAATCCT ACCATACAGA |
| 6441 | AAGACGTTCT TGAGTGTAAT GTGAAAACTA CCGAAGTTGT |
| 6481 | AGGAGACATT ATACTTAAAC CAGCAAATAA TAGTTTAAAA |
| 6521 | ATTACAGAAG AGGTTGGCCA CACAGATCTA ATGGCTGCTT |
| 6561 | ATGTAGACAA TTCTAGTCTT ACTATTAAGA AACCTAATGA |
| 6601 | ATTATCTAGA GTATTAGGTT TGAAAACCCT TGCTACTCAT |
| 6641 | GGTTTAGCTG CTGTTAATAG TGTCCCTTGG GATACTATAG |
| 6681 | CTAATTATGC TAAGCCTTTT CTTAACAAAG TTGTTAGTAC |
| 6721 | AACTACTAAC ATAGTTACAC GGTGTTTAAA CCGTGTTTGT |
| 6761 | ACTAATTATA TGCCTTATTT CTTTACTTTA TTGCTACAAT |
| 6801 | TGTGTACTTT TACTAGAAGT ACAAATTCTA GAATTAAAGC |
| 6841 | ATCTATGCCG ACTACTATAG CAAAGAATAC TGTTAAGAGT |
| 6881 | GTCGGTAAAT TTTGTCTAGA GGCTTCATTT AATTATTTGA |
| 6921 | AGTCACCTAA TTTTTCTAAA CTGATAAATA TTATAATTTG |
| 6961 | GTTTTTACTA TTAAGTGTTT GCCTAGGTTC TTTAATCTAC |
| 7001 | TCAACCGCTG CTTTAGGTGT TTTAATGTCT AATTTAGGCA |
| 7041 | TGCCTTCTTA CTGTACTGGT TACAGAGAAG GCTATTTGAA |
| 7081 | CTCTACTAAT GTCACTATTG CAACCTACTG TACTGGTTCT |
| 7121 | ATACCTTGTA GTGTTTGTCT TAGTGGTTTA GATTCTTTAG |
| 7161 | ACACCTATCC TTCTTTAGAA ACTATACAAA TTACCATTTC |
| 7201 | ATCTTTTAAA TGGGATTTAA CTGCTTTTGG CTTAGTTGCA |
| 7241 | GAGTGGTTTT TGGCATATAT TCTTTTCACT AGGTTTTTCT |
| 7281 | ATGTACTTGG ATTGGCTGCA ATCATGCAAT TGTTTTTCAG |
| 7321 | CTATTTTGCA GTACATTTTA TTAGTAATTC TTGGCTTATG |
| 7361 | TGGTTAATAA TTAATCTTGT ACAAATGGCC CCGATTTCAG |
| 7401 | CTATGGTTAG AATGTACATC TTCTTTGCAT CATTTTATTA |
| 7441 | TGTATGGAAA AGTTATGTGC ATGTTGTAGA CGGTTGTAAT |
| 7481 | TCATCAACTT GTATGATGTG TTACAAACGT AATAGAGCAA |
| 7521 | CAAGAGTCGA ATGTACAACT ATTGTTAATG GTGTTAGAAG |
| 7561 | GTCCTTTTAT GTCTATGCTA ATGGAGGTAA AGGCTTTTGC |
| 7601 | AAACTACACA ATTGGAATTG TGTTAATTGT GATACATTCT |
| 7641 | GTGCTGGTAG TACATTTATT AGTGATGAAG TTGCGAGAGA |
| 7681 | CTTGTCACTA CAGTTTAAAA GACCAATAAA TCCTACTGAC |
| 7721 | CAGTCTTCTT ACATCGTTGA TAGTGTTACA GTGAAGAATG |
| 7761 | GTTCCATCCA TCTTTACTTT GATAAAGCTG GTCAAAAGAC |
| 7801 | TTATGAAAGA CATTCTCTCT CTCATTTTGT TAACTTAGAC |
| 7841 | AACCTGAGAG CTAATAACAC TAAAGGTTCA TTGCCTATTA |
| 7881 | ATGTTATAGT TTTTGATGGT AAATCAAAAT GTGAAGAATC |
| 7921 | ATCTGCAAAA TCAGCGTCTG TTTACTACAG TCAGCTTATG |
| 7961 | TGTCAACCTA TACTGTTACT AGATCAGGCA TTAGTGTCTG |
| 8001 | ATGTTGGTGA TAGTGCGGAA GTTGCAGTTA AAATGTTTGA |
| 8041 | TGCTTACGTT AATACGTTTT CATCAACTTT TAACGTACCA |
| 8081 | ATGGAAAAAC TCAAAACACT AGTTGCAACT GCAGAAGCTG |
| 8121 | AACTTGCAAA GAATGTGTCC TTAGACAATG TCTTATCTAC |
| 8161 | TTTTATTTCA GCAGCTCGGC AAGGGTTTGT TGATTCAGAT |
| 8201 | GTAGAAACTA AAGATGTTGT TGAATGTCTT AAATTGTCAC |
| 8241 | ATCAATCTGA CATAGAAGTT ACTGGCGATA GTTGTAATAA |
| 8281 | CTATATGCTC ACCTATAACA AAGTTGAAAA CATGACACCC |
| 8321 | CGTGACCTTG GTGCTTGTAT TGACTGTAGT GCGCGTCATA |
| 8361 | TTAATGCGCA GGTAGCAAAA AGTCACAACA TTGCTTTGAT |
| 8401 | ATGGAACGTT AAAGATTTCA TGTCATTGTC TGAACAACTA |
| 8441 | CGAAAACAAA TACGTAGTGC TGCTAAAAAG AATAACTTAC |
| 8481 | CTTTTAAGTT GACATGTGCA ACTACTAGAC AAGTTGTTAA |
| 8521 | TGTTGTAACA ACAAAGATAG CACTTAAGGG TGGTAAAATT |
| 8561 | GTTAATAATT GGTTGAAGCA GTTAATTAAA GTTACACTTG |
| 8601 | TGTTCCTTTT TGTTGCTGCT ATTTTCTATT TAATAACACC |
| 8641 | TGTTCATGTC ATGTCTAAAC ATACTGACTT TTCAAGTGAA |
| 8681 | ATCATAGGAT ACAAGGCTAT TGATGGTGGT GTCACTCGTG |
| 8721 | ACATAGCATC TACAGATACT TGTTTTGCTA ACAAACATGC |
| 8761 | TGATTTTGAC ACATGGTTTA GCCAGCGTGG TGGTAGTTAT |
| 8801 | ACTAATGACA AAGCTTGCCC ATTGATTGCT GCAGTCATAA |
| 8841 | CAAGAGAAGT GGGTTTTGTC GTGCCTGGTT TGCCTGGCAC |
| 8881 | GATATTACGC ACAACTAATG GTGACTTTTT GCATTTCTTA |
| 8921 | CCTAGAGTTT TTAGTGCAGT TGGTAACATC TGTTACACAC |
| 8961 | CATCAAAACT TATAGAGTAC ACTGACTTTG CAACATCAGC |
| 9001 | TTGTGTTTTG GCTGCTGAAT GTACAATTTT TAAAGATGCT |
| 9041 | TCTGGTAAGC CAGTACCATA TTGTTATGAT ACCAATGTAC |
| 9081 | TAGAAGGTTC TGTTGCTTAT GAAAGTTTAC GCCCTGACAC |
| 9121 | ACGTTATGTG CTCATGGATG GCTCTATTAT TCAATTTCCT |
| 9161 | AACACCTACC TTGAAGGTTC TGTTAGAGTG GTAACAACTT |
| 9201 | TTGATTCTGA GTACTGTAGG CACGGCACTT GTGAAAGATC |
| 9241 | AGAAGCTGGT GTTTGTGTAT CTACTAGTGG TAGATGGGTA |
| 9281 | CTTAACAATG ATTATTACAG ATCTTTACCA GGAGTTTTCT |
| 9321 | GTGGTGTAGA TGCTGTAAAT TTACTTACTA ATATGTTTAC |
| 9361 | ACCACTAATT CAACCTATTG GTGCTTTGGA CATATCAGCA |
| 9401 | TCTATAGTAG CTGGTGGTAT TGTAGCTATC GTAGTAACAT |
| 9441 | GCCTTGCCTA CTATTTTATG AGGTTTAGAA GAGCTTTTGG |
| 9481 | TGAATACAGT CATGTAGTTG CCTTTAATAC TTTACTATTC |
| 9521 | CTTATGTCAT TCACTGTACT CTGTTTAACA CCAGTTTACT |
| 9561 | CATTCTTACC TGGTGTTTAT TCTGTTATTT ACTTGTACTT |
| 9601 | GACATTTTAT CTTACTAATG ATGTTTCTTT TTTAGCACAT |
| 9641 | ATTCAGTGGA TGGTTATGTT CACACCTTTA GTACCTTTCT |
| 9681 | GGATAACAAT TGCTTATATC ATTTGTATTT CCACAAAGCA |
| 9721 | TTTCTATTGG TTCTTTAGTA ATTACCTAAA GAGACGTGTA |
| 9761 | GTCTTTAATG GTGTTTCCTT TAGTACTTTT GAAGAAGCTG |
| 9801 | CGCTGTGCAC CTTTTTGTTA AATAAAGAAA TGTATCTAAA |
| 9841 | GTTGCGTAGT GATGTGCTAT TACCTCTTAC GCAATATAAT |
| 9881 | AGATACTTAG CTCTTTATAA TAAGTACAAG TATTTTAGTG |
| 9921 | GAGCAATGGA TACAACTAGC TACAGAGAAG CTGCTTGTTG |
| 9961 | TCATCTCGCA AAGGCTCTCA ATGACTTCAG TAACTCAGGT |
| 10001 | TCTGATGTTC TTTACCAACC ACCACAAACC TCTATCACCT |
| 10041 | CAGCTGTTTT GCAGAGTGGT TTTAGAAAAA TGGCATTCCC |
| 10081 | ATCTGGTAAA GTTGAGGGTT GTATGGTACA AGTAACTTGT |
| 10121 | GGTACAACTA CACTTAACGG TCTTTGGCTT GATGACGTAG |
| 10161 | TTTACTGTCC AAGACATGTG ATCTGCACCT CTGAAGACAT |
| 10201 | GCTTAACCCT AATTATGAAG ATTTACTCAT TCGTAAGTCT |
| 10241 | AATCATAATT TCTTGGTACA GGCTGGTAAT GTTCAACTCA |
| 10281 | GGGTTATTGG ACATTCTATG CAAAATTGTG TACTTAAGCT |
| 10321 | TAAGGTTGAT ACAGCCAATC CTAAGACACC TAAGTATAAG |
| 10361 | TTTGTTCGCA TTCAACCAGG ACAGACTTTT TCAGTGTTAG |
| 10401 | CTTGTTACAA TGGTTCACCA TCTGGTGTTT ACCAATGTGC |
| 10441 | TATGAGGCCC AATTTCACTA TTAAGGGTTC ATTCCTTAAT |
| 10481 | GGTTCATGTG GTAGTGTTGG TTTTAACATA GATTATGACT |
| 10521 | GTGTCTCTTT TTGTTACATG CACCATATGG AATTACCAAC |
| 10561 | TGGAGTTCAT GCTGGCACAG ACTTAGAAGG TAACTTTTAT |
| 10601 | GGACCTTTTG TTGACAGGCA AACAGCACAA GCAGCTGGTA |
| 10641 | CGGACACAAC TATTACAGTT AATGTTTTAG CTTGGTTGTA |
| 10681 | CGCTGCTGTT ATAAATGGAG ACAGGTGGTT TCTCAATCGA |
| 10721 | TTTACCACAA CTCTTAATGA CTTTAACCTT GTGGCTATGA |
| 10761 | AGTACAATTA TGAACCTCTA ACACAAGACC ATGTTGACAT |
| 10801 | ACTAGGACCT CTTTCTGCTC AAACTGGAAT TGCCGTTTTA |
| 10841 | GATATGTGTG CTTCATTAAA AGAATTACTG CAAAATGGTA |
| 10881 | TGAATGGACG TACCATATTG GGTAGTGCTT TATTAGAAGA |
| 10921 | TGAATTTACA CCTTTTGATG TTGTTAGACA ATGCTCAGGT |
| 10961 | GTTACTTTCC AAAGTGCAGT GAAAAGAACA ATCAAGGGTA |
| 11001 | CACACCACTG GTTGTTACTC ACAATTTTGA CTTCACTTTT |
| 11041 | AGTTTTAGTC CAGAGTACTC AATGGTCTTT GTTCTTTTTT |
| 11081 | TTGTATGAAA ATGCCTTTTT ACCTTTTGCT ATGGGTATTA |
| 11121 | TTGCTATGTC TGCTTTTGCA ATGATGTTTG TCAAACATAA |
| 11161 | GCATGCATTT CTCTGTTTGT TTTTGTTACC TTCTCTTGCC |
| 11201 | ACTGTAGCTT ATTTTAATAT GGTCTATATG CCTGCTAGTT |
| 11241 | GGGTGATGCG TATTATGACA TGGTTGGATA TGGTTGATAC |
| 11281 | TAGTTTGTCT GGTTTTAAGC TAAAAGACTG TGTTATGTAT |
| 11321 | GCATCAGCTG TAGTGTTACT AATCCTTATG ACAGCAAGAA |
| 11361 | CTGTGTATGA TGATGGTGCT AGGAGAGTGT GGACACTTAT |
| 11401 | GAATGTCTTG ACACTCGTTT ATAAAGTTTA TTATGGTAAT |
| 11441 | GCTTTAGATC AAGCCATTTC CATGTGGGCT CTTATAATCT |
| 11481 | CTGTTACTTC TAACTACTCA GGTGTAGTTA CAACTGTCAT |
| 11521 | GTTTTTGGCC AGAGGTATTG TTTTTATGTG TGTTGAGTAT |
| 11561 | TGCCCTATTT TCTTCATAAC TGGTAATACA CTTCAGTGTA |
| 11601 | TAATGCTAGT TTATTGTTTC TTAGGCTATT TTTGTACTTG |
| 11641 | TTACTTTGGC CTCTTTTGTT TACTCAACCG CTACTTTAGA |
| 11681 | CTGACTCTTG GTGTTTATGA TTACTTAGTT TCTACACAGG |
| 11721 | AGTTTAGATA TATGAATTCA CAGGGACTAC TCCCACCCAA |
| 11761 | GAATAGCATA GATGCCTTCA AACTCAACAT TAAATTGTTG |
| 11801 | GGTGTTGGTG GCAAACCTTG TATCAAAGTA GCCACTGTAC |
| 11841 | AGTCTAAAAT GTCAGATGTA AAGTGCACAT CAGTAGTCTT |
| 11881 | ACTCTCAGTT TTGCAACAAC TCAGAGTAGA ATCATCATCT |
| 11921 | AAATTGTGGG CTCAATGTGT CCAGTTACAC AATGACATTC |
| 11961 | TCTTAGCTAA AGATACTACT GAAGCCTTTG AAAAAATGGT |
| 12001 | TTCACTACTT TCTGTTTTGC TTTCCATGCA GGGTGCTGTA |
| 12041 | GACATAAACA AGCTTTGTGA AGAAATGCTG GACAACAGGG |
| 12081 | CAACCTTACA AGCTATAGCC TCAGAGTTTA GTTCCCTTCC |
| 12121 | ATCATATGCA GCTTTTGCTA CTGCTCAAGA AGCTTATGAG |
| 12161 | CAGGCTGTTG CTAATGGTGA TTCTGAAGTT GTTCTTAAAA |
| 12201 | AGTTGAAGAA GTCTTTGAAT GTGGCTAAAT CTGAATTTGA |
| 12241 | CCGTGATGCA GCCATGCAAC GTAAGTTGGA AAAGATGGCT |
| 12281 | GATCAAGCTA TGACCCAAAT GTATAAACAG GCTAGATCTG |
| 12321 | AGGACAAGAG GGCAAAAGTT ACTAGTGCTA TGCAGACAAT |
| 12361 | GCTTTTCACT ATGCTTAGAA AGTTGGATAA TGATGCACTC |
| 12401 | AACAACATTA TCAACAATGC AAGAGATGGT TGTGTTCCCT |
| 12441 | TGAACATAAT ACCTCTTACA ACAGCAGCCA AACTAATGGT |
| 12481 | TGTCATACCA GACTATAACA CATATAAAAA TACGTGTGAT |
| 12521 | GGTACAACAT TTACTTATGC ATCAGCATTG TGGGAAATCC |
| 12561 | AACAGGTTGT AGATGCAGAT AGTAAAATTG TTCAACTTAG |
| 12601 | TGAAATTAGT ATGGACAATT CACCTAATTT AGCATGGCCT |
| 12641 | CTTATTGTAA CAGCTTTAAG GGCCAATTCT GCTGTCAAAT |
| 12681 | TACAGAATAA TGAGCTTAGT CCTGTTGCAC TACGACAGAT |
| 12721 | GTCTTGTGCT GCCGGTACTA CACAAACTGC TTGCACTGAT |
| 12761 | GACAATGCGT TAGCTTACTA CAACACAACA AAGGGAGGTA |
| 12801 | GGTTTGTACT TGCACTGTTA TCCGATTTAC AGGATTTGAA |
| 12841 | ATGGGCTAGA TTCCCTAAGA GTGATGGAAC TGGTACTATC |
| 12881 | TATACAGAAC TGGAACCACC TTGTAGGTTT GTTACAGACA |
| 12921 | CACCTAAAGG TCCTAAAGTG AAGTATTTAT ACTTTATTAA |
| 12961 | AGGATTAAAC AACCTAAATA GAGGTATGGT ACTTGGTAGT |
| 13001 | TTAGCTGCCA CAGTACGTCT ACAAGCTGGT AATGCAACAG |
| 13041 | AAGTGCCTGC CAATTCAACT GTATTATCTT TCTGTGCTTT |
| 13081 | TGCTGTAGAT GCTGCTAAAG CTTACAAAGA TTATCTAGCT |
| 13121 | AGTGGGGGAC AACCAATCAC TAATTGTGTT AAGATGTTGT |
| 13161 | GTACACACAC TGGTACTGGT CAGGCAATAA CAGTTACACC |
| 13201 | GGAAGCCAAT ATGGATCAAG AATCCTTTGG TGGTGCATCG |
| 13241 | TGTTGTCTGT ACTGCCGTTG CCACATAGAT CATCCAAATC |
| 13281 | CTAAAGGATT TTGTGACTTA AAAGGTAAGT ATGTACAAAT |
| 13321 | ACCTACAACT TGTGCTAATG ACCCTGTGGG TTTTACACTT |
| 13361 | AAAAACACAG TCTGTACCGT CTGCGGTATG TGGAAAGGTT |
| 13401 | ATGGCTGTAG TTGTGATCAA CTCCGCGAAC CCATGCTTCA |
| 13441 | GTCAGCTGAT GCACAATCGT TTTTAAACGG GTTTGCGGTG |
| 13481 | TAAGTGCAGC CCGTCTTACA CCGTGCGGCA CAGGCACTAG |
| 13521 | TACTGATGTC GTATACAGGG CTTTTGACAT CTACAATGAT |
| 13561 | AAAGTAGCTG GTTTTGCTAA ATTCCTAAAA ACTAATTGTT |
| 13601 | GTCGCTTCCA AGAAAAGGAC GAAGATGACA ATTTAATTGA |
| 13641 | TTCTTACTTT GTAGTTAAGA GACACACTTT CTCTAACTAC |
| 13681 | CAACATGAAG AAACAATTTA TAATTTACTT AAGGATTGTC |
| 13721 | CAGCTGTTGC TAAACATGAC TTCTTTAAGT TTAGAATAGA |
| 13761 | CGGTGACATG GTACCACATA TATCACGTCA ACGTCTTACT |
| 13801 | AAATACACAA TGGCAGACCT CGTCTATGCT TTAAGGCATT |
| 13841 | TTGATGAAGG TAATTGTGAC ACATTAAAAG AAATACTTGT |
| 13881 | CACATACAAT TGTTGTGATG ATGATTATTT CAATAAAAAG |
| 13921 | GACTGGTATG ATTTTGTAGA AAACCCAGAT ATATTACGCG |
| 13961 | TATACGCCAA CTTAGGTGAA CGTGTACGCC AAGCTTTGTT |
| 14001 | AAAAACAGTA CAATTCTGTG ATGCCATGCG AAATGCTGGT |
| 14041 | ATTGTTGGTG TACTGACATT AGATAATCAA GATCTCAATG |
| 14081 | GTAACTGGTA TGATTTCGGT GATTTCATAC AAACCACGCC |
| 14121 | AGGTAGTGGA GTTCCTGTTG TAGATTCTTA TTATTCATTG |
| 14161 | TTAATGCCTA TATTAACCTT GACCAGGGCT TTAACTGCAG |
| 14201 | AGTCACATGT TGACACTGAC TTAACAAAGC CTTACATTAA |
| 14241 | GTGGGATTTG TTAAAATATG ACTTCACGGA AGAGAGGTTA |
| 14281 | AAACTCTTTG ACCGTTATTT TAAATATTGG GATCAGACAT |
| 14321 | ACCACCCAAA TTGTGTTAAC TGTTTGGATG ACAGATGCAT |
| 14361 | TCTGCATTGT GCAAACTTTA ATGTTTTATT CTCTACAGTG |
| 14401 | TTCCCACCTA CAAGTTTTGG ACCACTAGTG AGAAAAATAT |
| 14441 | TTGTTGATGG TGTTCCATTT GTAGTTTCAA CTGGATACCA |
| 14481 | CTTCAGAGAG CTAGGTGTTG TACATAATCA GGATGTAAAC |
| 14521 | TTACATAGCT CTAGACTTAG TTTTAAGGAA TTACTTGTGT |
| 14561 | ATGCTGCTGA CCCTGCTATG CACGCTGCTT CTGGTAATCT |
| 14601 | ATTACTAGAT AAACGCACTA CGTGCTTTTC AGTAGCTGCA |
| 14641 | CTTACTAACA ATGTTGCTTT TCAAACTGTC AAACCCGGTA |
| 14681 | ATTTTAACAA AGACTTCTAT GACTTTGCTG TGTCTAAGGG |
| 14721 | TTTCTTTAAG GAAGGAAGTT CTGTTGAATT AAAACACTTC |
| 14761 | TTCTTTGCTC AGGATGGTAA TGCTGCTATC AGCGATTATG |
| 14801 | ACTACTATCG TTATAATCTA CCAACAATGT GTGATATCAG |
| 14841 | ACAACTACTA TTTGTAGTTG AAGTTGTTGA TAAGTACTTT |
| 14881 | GATTGTTACG ATGGTGGCTG TATTAATGCT AACCAAGTCA |
| 14921 | TCGTCAACAA CCTAGACAAA TCAGCTGGTT TTCCATTTAA |
| 14961 | TAAATGGGGT AAGGCTAGAC TTTATTATGA TTCAATGAGT |
| 15001 | TATGAGGATC AAGATGCACT TTTCGCATAT ACAAAACGTA |
| 15041 | ATGTCATCCC TACTATAACT CAAATGAATC TTAAGTATGC |
| 15081 | CATTAGTGCA AAGAATAGAG CTCGCACCGT AGCTGGTGTC |
| 15121 | TCTATCTGTA GTACTATGAC CAATAGACAG TTTCATCAAA |
| 15161 | AATTATTGAA ATCAATAGCC GCCACTAGAG GAGCTACTGT |
| 15201 | AGTAATTGGA ACAAGCAAAT TCTATGGTGG TTGGCACAAC |
| 15241 | ATGTTAAAAA CTGTTTATAG TGATGTAGAA AACCCTCACC |
| 15281 | TTATGGGTTG GGATTATCCT AAATGTGATA GAGCCATGCC |
| 15321 | TAACATGCTT AGAATTATGG CCTCACTTGT TCTTGCTCGC |
| 15361 | AAACATACAA CGTGTTGTAG CTTGTCACAC CGTTTCTATA |
| 15401 | GATTAGCTAA TGAGTGTGCT CAAGTATTGA GTGAAATGGT |
| 15441 | CATGTGTGGC GGTTCACTAT ATGTTAAACC AGGTGGAACC |
| 15481 | TCATCAGGAG ATGCCACAAC TGCTTATGCT AATAGTGTTT |
| 15521 | TTAACATTTG TCAAGCTGTC ACGGCCAATG TTAATGCACT |
| 15561 | TTTATCTACT GATGGTAACA AAATTGCCGA TAAGTATGTC |
| 15601 | CGCAATTTAC AACACAGACT TTATGAGTGT CTCTATAGAA |
| 15641 | ATAGAGATGT TGACACAGAC TTTGTGAATG AGTTTTACGC |
| 15681 | ATATTTGCGT AAACATTTCT CAATGATGAT ACTCTCTGAC |
| 15721 | GATGCTGTTG TGTGTTTCAA TAGCACTTAT GCATCTCAAG |
| 15761 | GTCTAGTGGC TAGCATAAAG AACTTTAAGT CAGTTCTTTA |
| 15801 | TTATCAAAAC AATGTTTTTA TGTCTGAAGC AAAATGTTGG |
| 15841 | ACTGAGACTG ACCTTACTAA AGGACCTCAT GAATTTTGCT |
| 15881 | CTCAACATAC AATGCTAGTT AAACAGGGTG ATGATTATGT |
| 15921 | GTACCTTCCT TACCCAGATC CATCAAGAAT CCTAGGGGCC |
| 15961 | GGCTGTTTTG TAGATGATAT CGTAAAAACA GATGGTACAC |
| 16001 | TTATGATTGA ACGGTTCGTG TCTTTAGCTA TAGATGCTTA |
| 16041 | CCCACTTACT AAACATCCTA ATCAGGAGTA TGCTGATGTC |
| 16081 | TTTCATTTGT ACTTACAATA CATAAGAAAG CTACATGATG |
| 16121 | AGTTAACAGG ACACATGTTA GACATGTATT CTGTTATGCT |
| 16161 | TACTAATGAT AACACTTCAA GGTATTGGGA ACCTGAGTTT |
| 16201 | TATGAGGCTA TGTACACACC GCATACAGTC TTACAGGCTG |
| 16241 | TTGGGGCTTG TGTTCTTTGC AATTCACAGA CTTCATTAAG |
| 16281 | ATGTGGTGCT TGCATACGTA GACCATTCTT ATGTTGTAAA |
| 16321 | TGCTGTTACG ACCATGTCAT ATCAACATCA CATAAATTAG |
| 16361 | TCTTGTCTGT TAATCCGTAT GTTTGCAATG CTCCAGGTTG |
| 16401 | TGATGTCACA GATGTGACTC AACTTTACTT AGGAGGTATG |
| 16441 | AGCTATTATT GTAAATCACA TAAACCACCC ATTAGTTTTC |
| 16481 | CATTGTGTGC TAATGGACAA GTTTTTGGTT TATATAAAAA |
| 16521 | TACATGTGTT GGTAGCGATA ATGTTACTGA CTTTAATGCA |
| 16561 | ATTGCAACAT GTGACTGGAC AAATGCTGGT GATTACATTT |
| 16601 | TAGCTAACAC CTGTACTGAA AGACTCAAGC TTTTTGCAGC |
| 16641 | AGAAACGCTC AAAGCTACTG AGGAGACATT TAAACTGTCT |
| 16681 | TATGGTATTG CTACTGTACG TGAAGTGCTG TCTGACAGAG |
| 16721 | AATTACATCT TTCATGGGAA GTTGGTAAAC CTAGACCACC |
| 16761 | ACTTAACCGA AATTATGTCT TTACTGGTTA TCGTGTAACT |
| 16801 | AAAAACAGTA AAGTACAAAT AGGAGAGTAC ACCTTTGAAA |
| 16841 | AAGGTGACTA TGGTGATGCT GTTGTTTACC GAGGTACAAC |
| 16881 | AACTTACAAA TTAAATGTTG GTGATTATTT TGTGCTGACA |
| 16921 | TCACATACAG TAATGCCATT AAGTGCACCT ACACTAGTGC |
| 16961 | CACAAGAGCA CTATGTTAGA ATTACTGGCT TATACCCAAC |
| 17001 | ACTCAATATC TCAGATGAGT TTTCTAGCAA TGTTGCAAAT |
| 17041 | TATCAAAAGG TTGGTATGCA AAAGTATTCT ACACTCCAGG |
| 17081 | GACCACCTGG TACTGGTAAG AGTCATTTTG CTATTGGCCT |
| 17121 | AGCTCTCTAC TACCCTTCTG CTCGCATAGT GTATACAGCT |
| 17161 | TGCTCTCATG CCGCTGTTGA TGCACTATGT GAGAAGGCAT |
| 17201 | TAAAATATTT GCCTATAGAT AAATGTAGTA GAATTATACC |
| 17241 | TGCACGTGCT CGTGTAGAGT GTTTTGATAA ATTCAAAGTG |
| 17281 | AATTCAACAT TAGAACAGTA TGTCTTTTGT ACTGTAAATG |
| 17321 | CATTGCCTGA GACGACAGCA GATATAGTTG TCTTTGATGA |
| 17361 | AATTTCAATG GCCACAAATT ATGATTTGAG TGTTGTCAAT |
| 17401 | GCCAGATTAC GTGCTAAGCA CTATGTGTAC ATTGGCGACC |
| 17441 | CTGCTCAATT ACCTGCACCA CGCACATTGC TAACTAAGGG |
| 17481 | CACACTAGAA CCAGAATATT TCAATTCAGT GTGTAGACTT |
| 17521 | ATGAAAACTA TAGGTCCAGA CATGTTCCTC GGAACTTGTC |
| 17561 | GGCGTTGTCC TGCTGAAATT GTTGACACTG TGAGTGCTTT |
| 17601 | GGTTTATGAT AATAAGCTTA AAGCACATAA AGACAAATCA |
| 17641 | GCTCAATGCT TTAAAATGTT TTATAAGGGT GTTATCACGC |
| 17681 | ATGATGTTTC ATCTGCAATT AACAGGCCAC AAATAGGCGT |
| 17721 | GGTAAGAGAA TTCCTTACAC GTAACCCTGC TTGGAGAAAA |
| 17761 | GCTGTCTTTA TTTCACCTTA TAATTCACAG AATGCTGTAG |
| 17801 | CCTCAAAGAT TTTGGGACTA CCAACTCAAA CTGTTGATTC |
| 17841 | ATCACAGGGC TCAGAATATG ACTATGTCAT ATTCACTCAA |
| 17881 | ACCACTGAAA CAGCTCACTC TTGTAATGTA AACAGATTTA |
| 17921 | ATGTTGCTAT TACCAGAGCA AAAGTAGGCA TACTTTGCAT |
| 17961 | AATGTCTGAT AGAGACCTTT ATGACAAGTT GCAATTTACA |
| 18001 | AGTCTTGAAA TTCCACGTAG GAATGTGGCA ACTTTACAAG |
| 18041 | CTGAAAATGT AACAGGACTC TTTAAAGATT GTAGTAAGGT |
| 18081 | AATCACTGGG TTACATCCTA CACAGGCACC TACACACCTC |
| 18121 | AGTGTTGACA CTAAATTCAA AACTGAAGGT TTATGTGTTG |
| 18161 | ACATACCTGG CATACCTAAG GACATGACCT ATAGAAGACT |
| 18201 | CATCTCTATG ATGGGTTTTA AAATGAATTA TCAAGTTAAT |
| 18241 | GGTTACCCTA ACATGTTTAT CACCCGCGAA GAAGCTATAA |
| 18281 | GACATGTACG TGCATGGATT GGCTTCGATG TCGAGGGGTG |
| 18321 | TCATGCTACT AGAGAAGCTG TTGGTACCAA TTTACCTTTA |
| 18361 | CAGCTAGGTT TTTCTACAGG TGTTAACCTA GTTGCTGTAC |
| 18401 | CTACAGGTTA TGTTGATACA CCTAATAATA CAGATTTTTC |
| 18441 | CAGAGTTAGT GCTAAACCAC CGCCTGGAGA TCAATTTAAA |
| 18481 | CACCTCATAC CACTTATGTA CAAAGGACTT CCTTGGAATG |
| 18521 | TAGTGCGTAT AAAGATTGTA CAAATGTTAA GTGACACACT |
| 18561 | TAAAAATCTC TCTGACAGAG TCGTATTTGT CTTATGGGCA |
| 18601 | CATGGCTTTG AGTTGACATC TATGAAGTAT TTTGTGAAAA |
| 18641 | TAGGACCTGA GCGCACCTGT TGTCTATGTG ATAGACGTGC |
| 18681 | CACATGCTTT TCCACTGCTT CAGACACTTA TGCCTGTTGG |
| 18721 | CATCATTCTA TTGGATTTGA TTACGTCTAT AATCCGTTTA |
| 18761 | TGATTGATGT TCAACAATGG GGTTTTACAG GTAACCTACA |
| 18801 | AAGCAACCAT GATCTGTATT GTCAAGTCCA TGGTAATGCA |
| 18841 | CATGTAGCTA GTTGTGATGC AATCATGACT AGGTGTCTAG |
| 18881 | CTGTCCACGA GTGCTTTGTT AAGCGTGTTG ACTGGACTAT |
| 18921 | TGAATATCCT ATAATTGGTG ATGAACTGAA GATTAATGCG |
| 18961 | GCTTGTAGAA AGGTTCAACA CATGGTTGTT AAAGCTGCAT |
| 19001 | TATTAGCAGA CAAATTCCCA GTTCTTCACG ACATTGGTAA |
| 19041 | CCCTAAAGCT ATTAAGTGTG TACCTCAAGC TGATGTAGAA |
| 19081 | TGGAAGTTCT ATGATGCACA GCCTTGTAGT GACAAAGCTT |
| 19121 | ATAAAATAGA AGAATTATTC TATTCTTATG CCACACATTC |
| 19161 | TGACAAATTC ACAGATGGTG TATGCCTATT TTGGAATTGC |
| 19201 | AATGTCGATA GATATCCTGC TAATTCCATT GTTTGTAGAT |
| 19241 | TTGACACTAG AGTGCTATCT AACCTTAACT TGCCTGGTTG |
| 19281 | TGATGGTGGC AGTTTGTATG TAAATAAACA TGCATTCCAC |
| 19321 | ACACCAGCTT TTGATAAAAG TGCTTTTGTT AATTTAAAAC |
| 19361 | AATTACCATT TTTCTATTAC TCTGACAGTC CATGTGAGTC |
| 19401 | TCATGGAAAA CAAGTAGTGT CAGATATAGA TTATGTACCA |
| 19441 | CTAAAGTCTG CTACGTGTAT AACACGTTGC AATTTAGGTG |
| 19481 | GTGCTGTCTG TAGACATCAT GCTAATGAGT ACAGATTGTA |
| 19521 | TCTCGATGCT TATAACATGA TGATCTCAGC TGGCTTTAGC |
| 19561 | TTGTGGGTTT ACAAACAATT TGATACTTAT AACCTCTGGA |
| 19601 | ACACTTTTAC AAGACTTCAG AGTTTAGAAA ATGTGGCTTT |
| 19641 | TAATGTTGTA AATAAGGGAC ACTTTGATGG ACAACAGGGT |
| 19681 | GAAGTACCAG TTTCTATCAT TAATAACACT GTTTACACAA |
| 19721 | AAGTTGATGG TGTTGATGTA GAATTGTTTG AAAATAAAAC |
| 19761 | AACATTACCT GTTAATGTAG CATTTGAGCT TTGGGCTAAG |
| 19801 | CGCAACATTA AACCAGTACC AGAGGTGAAA ATACTCAATA |
| 19841 | ATTTGGGTGT GGACATTGCT GCTAATACTG TGATCTGGGA |
| 19881 | CTACAAAAGA GATGCTCCAG CACATATATC TACTATTGGT |
| 19921 | GTTTGTTCTA TGACTGACAT AGCCAAGAAA CCAACTGAAA |
| 19961 | CGATTTGTGC ACCACTCACT GTCTTTTTTG ATGGTAGAGT |
| 20001 | TGATGGTCAA GTAGACTTAT TTAGAAATGC CCGTAATGGT |
| 20041 | GTTCTTATTA CAGAAGGTAG TGTTAAAGGT TTACAACCAT |
| 20081 | CTGTAGGTCC CAAACAAGCT AGTCTTAATG GAGTCACATT |
| 20121 | AATTGGAGAA GCCGTAAAAA CACAGTTCAA TTATTATAAG |
| 20161 | AAAGTTGATG GTGTTGTCCA ACAATTACCT GAAACTTACT |
| 20201 | TTACTCAGAG TAGAAATTTA CAAGAATTTA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TTTACATCTA CTGATTGGAC TAGCTAAACG TTTTAAGGAA |
| 20401 | TCACCTTTTG AATTAGAAGA TTTTATTCCT ATGGACAGTA |
| 20441 | CAGTTAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCTA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTTTGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGTTTATAC AACAAAAGCT AGCTCTTGGA GGTTCCGTGG |
| 21161 | CTATAAAGAT AACAGAACAT TCTTGGAATG CTGATCTTTA |
| 21201 | TAAGCTCATG GGACACTTCG CATGGTGGAC AGCCTTTGTT |
| 21241 | ACTAATGTGA ATGCGTCATC ATCTGAAGCA TTTTTAATTG |
| 21281 | GATGTAATTA TCTTGGCAAA CCACGCGAAC AAATAGATGG |
| 21321 | TTATGTCATG CATGCAAATT ACATATTTTG GAGGAATACA |
| 21361 | AATCCAATTC AGTTGTCTTC CTATTCTTTA TTTGACATGA |
| 21401 | GTAAATTTCC CCTTAAATTA AGGGGTACTG CTGTTATGTC |
| 21441 | TTTAAAAGAA GGTCAAATCA ATGATATGAT TTTATCTCTT |
| 21481 | CTTAGTAAAG GTAGACTTAT AATTAGAGAA AACAACAGAG |
| 21521 | TTGTTATTTC TAGTGATGTT CTTGTTAACA ACTAAACGAA |
| 21561 | CAATGTTTGT TTTTCTTGTT TTATTGCCAC TAGTCTCTAG |
| 21601 | TCAGTGTGTT AATCTTACAA CCAGAACTCA ATTACCCCCT |
| 21641 | GCATACACTA ATTCTTTCAC ACGTGGTGTT TATTACCCTG |
| 21681 | ACAAAGTTTT CAGATCCTCA GTTTTACATT CAACTCAGGA |
| 21721 | CTTGTTCTTA CCTTTCTTTT CCAATGTTAC TTGGTTCCAT |
| 21761 | GCTATACATG TCTCTGGGAC CAATGGTACT AAGAGGTTTG |
| 21801 | ATAACCCTGT CCTACCATTT AATGATGGTG TTTATTTTGC |
| 21841 | TTCCACTGAG AAGTCTAACA TAATAAGAGG CTGGATTTTT |
| 21881 | GGTACTACTT TAGATTCGAA GACCCAGTCC CTACTTATTG |
| 21921 | TTAATAACGC TACTAATGTT GTTATTAAAG TCTGTGAATT |
| 21961 | TCAATTTTGT AATGATCCAT TTTTGGGTGT TTATTACCAC |
| 22001 | AAAAACAACA AAAGTTGGAT GGAAAGTGAG TTCAGAGTTT |
| 22041 | ATTCTAGTGC GAATAATTGC ACTTTTGAAT ATGTCTCTCA |
| 22081 | GCCTTTTCTT ATGGACCTTG AAGGAAAACA GGGTAATTTC |
| 22121 | AAAAATCTTA GGGAATTTGT GTTTAAGAAT ATTGATGGTT |
| 22161 | ATTTTAAAAT ATATTCTAAG CACACGCCTA TTAATTTAGT |
| 22201 | GCGTGATCTC CCTCAGGGTT TTTCGGCTTT AGAACCATTG |
| 22241 | GTAGATTTGC CAATAGGTAT TAACATCACT AGGTTTCAAA |
| 22281 | CTTTACTTGC TTTACATAGA AGTTATTTGA CTCCTGGTGA |
| 22321 | TTCTTCTTCA GGTTGGACAG CTGGTGCTGC AGCTTATTAT |
| 22361 | GTGGGTTATC TTCAACCTAG GACTTTTCTA TTAAAATATA |
| 22401 | ATGAAAATGG AACCATTACA GATGCTGTAG ACTGTGCACT |
| 22441 | TGACCCTCTC TCAGAAACAA AGTGTACGTT GAAATCCTTC |
| 22481 | ACTGTAGAAA AAGGAATCTA TCAAACTTCT AACTTTAGAG |
| 22521 | TCCAACCAAC AGAATCTATT GTTAGATTTC CTAATATTAC |
| 22561 | AAACTTGTGC CCTTTTGGTG AAGTTTTTAA CGCCACCAGA |
| 22601 | TTTGCATCTG TTTATGCTTG GAACAGGAAG AGAATCAGCA |
| 22641 | ACTGTGTTGC TGATTATTCT GTCCTATATA ATTCCGCATC |
| 22681 | ATTTTCCACT TTTAAGTGTT ATGGAGTGTC TCCTACTAAA |
| 22721 | TTAAATGATC TCTGCTTTAC TAATGTCTAT GCAGATTCAT |
| 22761 | TTGTAATTAG AGGTGATGAA GTCAGACAAA TCGCTCCAGG |
| 22801 | GCAAACTGGA AAGATTGCTG ATTATAATTA TAAATTACCA |
| 22841 | GATGATTTTA CAGGCTGCGT TATAGCTTGG AATTCTAACA |
| 22881 | ATCTTGATTC TAAGGTTGGT GGTAATTATA ATTACCTGTA |
| 22921 | TAGATTGTTT AGGAAGTCTA ATCTCAAACC TTTTGAGAGA |
| 22961 | GATATTTCAA CTGAAATCTA TCAGGCCGGT AGCACACCTT |
| 23001 | GTAATGGTGT TGAAGGTTTT AATTGTTACT TTCCTTTACA |
| 23041 | ATCATATGGT TTCCAACCCA CTAATGGTGT TGGTTACCAA |
| 23081 | CCATACAGAG TAGTAGTACT TTCTTTTGAA CTTCTACATG |
| 23121 | CACCAGCAAC TGTTTGTGGA CCTAAAAAGT CTACTAATTT |
| 23161 | GGTTAAAAAC AAATGTGTCA ATTTCAACTT CAATGGTTTA |
| 23201 | ACAGGCACAG GTGTTCTTAC TGAGTCTAAC AAAAAGTTTC |
| 23241 | TGCCTTTCCA ACAATTTGGC AGAGACATTG CTGACACTAC |
| 23281 | TGATGCTGTC CGTGATCCAC AGACACTTGA GATTCTTGAC |
| 23321 | ATTACACCAT GTTCTTTTGG TGGTGTCAGT GTTATAACAC |
| 23361 | CAGGAACAAA TACTTCTAAC CAGGTTGCTG TTCTTTATCA |
| 23401 | GGATGTTAAC TGCACAGAAG TCCCTGTTGC TATTCATGCA |
| 23441 | GATCAACTTA CTCCTACTTG GCGTGTTTAT TCTACAGGTT |
| 23481 | CTAATGTTTT TCAAACACGT GCAGGCTGTT TAATAGGGGC |
| 23521 | TGAACATGTC AACAACTCAT ATGAGTGTGA CATACCCATT |
| 23561 | GGTGCAGGTA TATGCGCTAG TTATCAGACT CAGACTAATT |
| 23601 | CTCCTCGGCG GGCACGTAGT GTAGCTAGTC AATCCATCAT |
| 23641 | TGCCTACACT ATGTCACTTG GTGCAGAAAA TTCAGTTGCT |
| 23681 | TACTCTAATA ACTCTATTGC CATACCCACA AATTTTACTA |
| 23721 | TTAGTGTTAC CACAGAAATT CTACCAGTGT CTATGACCAA |
| 23761 | GACATCAGTA GATTGTACAA TGTACATTTG TGGTGATTCA |
| 23801 | ACTGAATGCA GCAATCTTTT GTTGCAATAT GGCAGTTTTT |
| 23841 | GTACACAATT AAACCGTGCT TTAACTGGAA TAGCTGTTGA |
| 23881 | ACAAGACAAA AACACCCAAG AAGTTTTTGC ACAAGTCAAA |
| 23921 | CAAATTTACA AAACACCACC AATTAAAGAT TTTGGTGGTT |
| 23961 | TTAATTTTTC ACAAATATTA CCAGATCCAT CAAAACCAAG |
| 24001 | CAAGAGGTCA TTTATTGAAG ATCTACTTTT CAACAAAGTG |
| 24041 | ACACTTGCAG ATGCTGGCTT CATCAAACAA TATGGTGATT |
| 24081 | GCCTTGGTGA TATTGCTGCT AGAGACCTCA TTTGTGCACA |
| 24121 | AAAGTTTAAC GGCCTTACTG TTTTGCCACC TTTGCTCACA |
| 24161 | GATGAAATGA TTGCTCAATA CACTTCTGCA CTGTTAGCGG |
| 24201 | GTACAATCAC TTCTGGTTGG ACCTTTGGTG CAGGTGCTGC |
| 24241 | ATTACAAATA CCATTTGCTA TGCAAATGGC TTATAGGTTT |
| 24281 | AATGGTATTG GAGTTACACA GAATGTTCTC TATGAGAACC |
| 24321 | AAAAATTGAT TGCCAACCAA TTTAATAGTG CTATTGGCAA |
| 24361 | AATTCAAGAC TCACTTTCTT CCACAGCAAG TGCACTTGGA |
| 24401 | AAACTTCAAG ATGTGGTCAA CCAAAATGCA CAAGCTTTAA |
| 24441 | ACACGCTTGT TAAACAACTT AGCTCCAATT TTGGTGCAAT |
| 24481 | TTCAAGTGTT TTAAATGATA TCCTTTCACG TCTTGACAAA |
| 24521 | GTTGAGGCTG AAGTGCAAAT TGATAGGTTG ATCACAGGCA |
| 24561 | GACTTCAAAG TTTGCAGACA TATGTGACTC AACAATTAAT |
| 24601 | TAGAGCTGCA GAAATCAGAG CTTCTGCTAA TCTTGCTGCT |
| 24641 | ACTAAAATGT CAGAGTGTGT ACTTGGACAA TCAAAAAGAG |
| 24681 | TTGATTTTTG TGGAAAGGGC TATCATCTTA TGTCCTTCCC |
| 24721 | TCAGTCAGCA CCTCATGGTG TAGTCTTCTT GCATGTGACT |
| 24761 | TATGTCCCTG CACAAGAAAA GAACTTCACA ACTGCTCCTG |
| 24801 | CCATTTGTCA TGATGGAAAA GCACACTTTC CTCGTGAAGG |
| 24841 | TGTCTTTGTT TCAAATGGCA CACACTGGTT TGTAACACAA |
| 24881 | AGGAATTTTT ATGAACCACA AATCATTACT ACAGACAACA |
| 24921 | CATTTGTGTC TGGTAACTGT GATGTTGTAA TAGGAATTGT |
| 24961 | CAACAACACA GTTTATGATC CTTTGCAACC TGAATTAGAC |
| 25001 | TCATTCAAGG AGGAGTTAGA TAAATATTTT AAGAATCATA |
| 25041 | CATCACCAGA TGTTGATTTA GGTGACATCT CTGGCATTAA |
| 25081 | TGCTTCAGTT GTAAACATTC AAAAAGAAAT TGACCGCCTC |
| 25121 | AATGAGGTTG CCAAGAATTT AAATGAATCT CTCATCGATC |
| 25161 | TCCAAGAACT TGGAAAGTAT GAGCAGTATA TAAAATGGCC |
| 25201 | ATGGTACATT TGGCTAGGTT TTATAGCTGG CTTGATTGCC |
| 25241 | ATAGTAATGG TGACAATTAT GCTTTGCTGT ATGACCAGTT |
| 25281 | GCTGTAGTTG TCTCAAGGGC TGTTGTTCTT GTGGATCCTG |
| 25321 | CTGCAAATTT GATGAAGACG ACTCTGAGCC AGTGCTCAAA |
| 25361 | GGAGTCAAAT TACATTACAC ATAAACGAAC TTATGGATTT |
| 25401 | GTTTATGAGA ATCTTCACAA TTGGAACTGT AACTTTGAAG |
| 25441 | CAAGGTGAAA TCAAGGATGC TACTCCTTCA GATTTTGTTC |
| 25481 | GCGCTACTGC AACGATACCG ATACAAGCCT CACTCCCTTT |
| 25521 | CGGATGGCTT ATTGTTGGCG TTGCACTTCT TGCTGTTTTT |
| 25561 | CAGAGCGCTT CCAAAATCAT AACCCTCAAA AAGAGATGGC |
| 25601 | AACTAGCACT CTCCAAGGGT GTTCACTTTG TTTGCAACTT |
| 25641 | GCTGTTGTTG TTTGTAACAG TTTACTCACA CCTTTTGCTC |
| 25681 | GTTGCTGCTG GCCTTGAAGC CCCTTTTCTC TATCTTTATG |
| 25721 | CTTTAGTCTA CTTCTTGCAG AGTATAAACT TTGTAAGAAT |
| 25761 | AATAATGAGG CTTTGGCTTT GCTGGAAATG CCGTTCCAAA |
| 25801 | AACCCATTAC TTTATGATGC CAACTATTTT CTTTGCTGGC |
| 25841 | ATACTAATTG TTACGACTAT TGTATACCTT ACAATAGTGT |
| 25881 | AACTTCTTCA ATTGTCATTA CTTCAGGTGA TGGCACAACA |
| 25921 | AGTCCTATTT CTGAACATGA CTACCAGATT GGTGGTTATA |
| 25961 | CTGAAAAATG GGAATCTGGA GTAAAAGACT GTGTTGTATT |
| 26001 | ACACAGTTAC TTCACTTCAG ACTATTACCA GCTGTACTCA |
| 26041 | ACTCAATTGA GTACAGACAC TGGTGTTGAA CATGTTACCT |
| 26081 | TCTTCATCTA CAATAAAATT GTTGATGAGC CTGAAGAACA |
| 26121 | TGTCCAAATT CACACAATCG ACGGTTCATC CGGAGTTGTT |
| 26161 | AATCCAGTAA TGGAACCAAT TTATGATGAA CCGACGACGA |
| 26201 | CTACTAGCGT GCCTTTGTAA GCACAAGCTG ATGAGTACGA |
| 26241 | ACTTATGTAC TCATTCGTTT CGGAAGAGAC AGGTACGTTA |
| 26281 | ATAGTTAATA GCGTACTTCT TTTTCTTGCT TTCGTGGTAT |
| 26321 | TCTTGCTAGT TACACTAGCC ATCCTTACTG CGCTTCGATT |
| 26361 | GTGTGCGTAC TGCTGCAATA TTGTTAACGT GAGTCTTGTA |
| 26401 | AAACCTTCTT TTTACGTTTA CTCTCGTGTT AAAAATCTGA |
| 26441 | ATTCTTCTAG AGTTCCTGAT CTTCTGGTCT AAACGAACTA |
| 26481 | AATATTATAT TAGTTTTTCT GTTTGGAACT TTAATTTTAG |
| 26521 | CCATGGCAGA TTCCAACGGT ACTATTACCG TTGAAGAGCT |
| 26561 | TAAAAAGCTC CTTGAACAAT GGAACCTAGT AATAGGTTTC |
| 26601 | CTATTCCTTA CATGGATTTG TCTTCTACAA TTTGCCTATG |
| 26641 | CCAACAGGAA TAGGTTTTTG TATATAATTA AGTTAATTTT |
| 26681 | CCTCTGGCTG TTATGGCCAG TAACTTTAGC TTGTTTTGTG |
| 26721 | CTTGCTGCTG TTTACAGAAT AAATTGGATC ACCGGTGGAA |
| 26761 | TTGCTATCGC AATGGCTTGT CTTGTAGGCT TGATGTGGCT |
| 26801 | CAGCTACTTC ATTGCTTCTT TCAGACTGTT TGCGCGTACG |
| 26841 | CGTTCCATGT GGTCATTCAA TCCAGAAACT AACATTCTTC |
| 26881 | TCAACGTGCC ACTCCATGGC ACTATTCTGA CCAGACCGCT |
| 26921 | TCTAGAAAGT GAACTCGTAA TCGGAGCTGT GATCCTTCGT |
| 26961 | GGACATCTTC GTATTGCTGG ACACCATCTA GGACGCTGTG |
| 27001 | ACATCAAGGA CCTGCCTAAA GAAATCACTG TTGCTACATC |
| 27041 | ACGAACGCTT TCTTATTACA AATTGGGAGC TTCGCAGCGT |
| 27081 | GTAGCAGGTG ACTCAGGTTT TGCTGCATAC AGTCGCTACA |
| 27121 | GGATTGGCAA CTATAAATTA AACACAGACC ATTCCAGTAG |
| 27161 | CAGTGACAAT ATTGCTTTGC TTGTACAGTA AGTGACAACA |
| 27201 | GATGTTTCAT CTCGTTGACT TTCAGGTTAC TATAGCAGAG |
| 27241 | ATATTACTAA TTATTATGAG GACTTTTAAA GTTTCCATTT |
| 27281 | GGAATCTTGA TTACATCATA AACCTCATAA TTAAAAATTT |
| 27321 | ATCTAAGTCA CTAACTGAGA ATAAATATTC TCAATTAGAT |
| 27361 | GAAGAGCAAC CAATGGAGAT TGATTAAACG AACATGAAAA |
| 27401 | TTATTCTTTT CTTGGCACTG ATAACACTCG CTACTTGTGA |
| 27441 | GCTTTATCAC TACCAAGAGT GTGTTAGAGG TACAACAGTA |
| 27481 | CTTTTAAAAG AACCTTGCTC TTCTGGAACA TACGAGGGCA |
| 27521 | ATTCACCATT TCATCCTCTA GCTGATAACA AATTTGCACT |
| 27561 | GACTTGCTTT AGCACTCAAT TTGCTTTTGC TTGTCCTGAC |
| 27601 | GGCGTAAAAC ACGTCTATCA GTTACGTGCC AGATCAGTTT |
| 27641 | CACCTAAACT GTTCATCAGA CAAGAGGAAG TTCAAGAACT |
| 27681 | TTACTCTCCA ATTTTTCTTA TTGTTGCGGC AATAGTGTTT |
| 27721 | ATAACACTTT GCTTCACACT CAAAAGAAAG ACAGAATGAT |
| 27761 | TGAACTTTCA TTAATTGACT TCTATTTGTG CTTTTTAGCC |
| 27801 | TTTCTGCTAT TCCTTGTTTT AATTATGCTT ATTATCTTTT |
| 27841 | GGTTCTCACT TGAACTGCAA GATCATAATG AAACTTGTCA |
| 27881 | CGCCTAAACG AACATGAAAT TTCTTGTTTT CTTAGGAATC |
| 27921 | ATCACAACTG TAGCTGCATT TCACCAAGAA TGTAGTTTAC |
| 27961 | AGTCATGTAC TCAACATCAA CCATATGTAG TTGATGACCC |
| 28001 | GTGTCCTATT CACTTCTATT CTAAATGGTA TATTAGAGTA |
| 28041 | GGAGCTAGAA AATCAGCACC TTTAATTGAA TTGTGCGTGG |
| 28081 | ATGAGGCTGG TTCTAAATCA CCCATTCAGT ACATCGATAT |
| 28121 | CGGTAATTAT ACAGTTTCCT GTTTACCTTT TACAATTAAT |
| 28161 | TGCCAGGAAC CTAAATTGGG TAGTCTTGTA GTGCGTTGTT |
| 28201 | CGTTCTATGA AGACTTTTTA GAGTATCATG ACGTTCGTGT |
| 28241 | TGTTTTAGAT TTCATCTAAA CGAACAAACT AAAATGTCTG |
| 28281 | ATAATGGACC CCAAAATCAG CGAAATGCAC CCCGCATTAC |
| 28321 | GTTTGGTGGA CCCTCAGATT CAACTGGCAG TAACCAGAAT |
| 28361 | GGAGAACGCA GTGGGGCGCG ATCAAAACAA CGTCGGCCCC |
| 28401 | AAGGTTTACC CAATAATACT GCGTCTTGGT TCACCGCTCT |
| 28441 | CACTCAACAT GGCAAGGAAG ACCTTAAATT CCCTCGAGGA |
| 28481 | CAAGGCGTTC CAATTAACAC CAATAGCAGT CCAGATGACC |
| 28521 | AAATTGGCTA CTACCGAAGA GCTACCAGAC GAATTCGTGG |
| 28561 | TGGTGACGGT AAAATGAAAG ATCTCAGTCC AAGATGGTAT |
| 28601 | TTCTACTACC TAGGAACTGG GCCAGAAGCT GGACTTCCCT |
| 28641 | ATGGTGCTAA CAAAGACGGC ATCATATGGG TTGCAACTGA |
| 28681 | GGGAGCCTTG AATACACCAA AAGATCACAT TGGCACCCGC |
| 28721 | AATCCTGCTA ACAATGCTGC AATCGTGCTA CAACTTCCTC |
| 28761 | AAGGAACAAC ATTGCCAAAA GGCTTCTACG CAGAAGGGAG |
| 28801 | CAGAGGCGGC AGTCAAGCCT CTTCTCGTTC CTCATCACGT |
| 28841 | AGTCGCAACA GTTCAAGAAA TTCAACTCCA GGCAGCAGTA |
| 28881 | GGGGAACTTC TCCTGCTAGA ATGGCTGGCA ATGGCGGTGA |
| 28921 | TGCTGCTCTT GCTTTGCTGC TGCTTGACAG ATTGAACCAG |
| 28961 | CTTGAGAGCA AAATGTCTGG TAAAGGCCAA CAACAACAAG |
| 29001 | GCCAAACTGT CACTAAGAAA TCTGCTGCTG AGGCTTCTAA |
| 29041 | GAAGCCTCGG CAAAAACGTA CTGCCACTAA AGCATACAAT |
| 29081 | GTAACACAAG CTTTCGGCAG ACGTGGTCCA GAACAAACCC |
| 29121 | AAGGAAATTT TGGGGACCAG GAACTAATCA GACAAGGAAC |
| 29161 | TGATTACAAA CATTGGCCGC AAATTGCACA ATTTGCCCCC |
| 29201 | AGCGCTTCAG CGTTCTTCGG AATGTCGCGC ATTGGCATGG |
| 29241 | AAGTCACACC TTCGGGAACG TGGTTGACCT ACACAGGTGC |
| 29281 | CATCAAATTG GATGACAAAG ATCCAAATTT CAAAGATCAA |
| 29321 | GTCATTTTGC TGAATAAGCA TATTGACGCA TACAAAACAT |
| 29361 | TCCCACCAAC AGAGCCTAAA AAGGACAAAA AGAAGAAGGC |
| 29401 | TGATGAAACT CAAGCCTTAC CGCAGAGACA GAAGAAACAG |
| 29441 | CAAACTGTGA CTCTTCTTCC TGCTGCAGAT TTGGATGATT |
| 29481 | TCTCCAAACA ATTGCAACAA TCCATGAGCA GTGCTGACTC |
| 29521 | AACTCAGGCC TAAACTCATG CAGACCACAC AAGGCAGATG |
| 29561 | GGCTATATAA ACGTTTTCGC TTTTCCGTTT ACGATATATA |
| 29601 | GTCTACTCTT GTGCAGAATG AATTCTCGTA ACTACATAGC |
| 29641 | ACAAGTAGAT GTAGTTAACT TTAATCTCAC ATAGCAATCT |
| 29681 | TTAATCAGTG TGTAACATTA GGGAGGACTT GAAAGAGCCA |
| 29721 | CCACATTTTC ACCGAGGCCA CGCGGAGTAC GATCGAGTGT |
| 29761 | ACAGTGAACA ATGCTAGGGA GAGCTGCCTA TATGGAAGAG |
| 29801 | CCCTAATGTG TAAAATTAAT TTTAGTAGTG CTATCCCCAT |
| 29841 | GTGATTTTAA TAGCTTCTTA GGAGAATGAC AAAAAAAAAA |
| 29881 | AAAAAAAAAA AAAAAAAAAA AAA |

The SARS-CoV-2 can have a 5′ untranslated region (5′ UTR; also known as a leader sequence or leader RNA) corresponding to positions 1-265 of SEQ II) NO: 1. Such a 5′ UTR can include the region of an mRNA that is directly upstream from the initiation codon. The 5′UTR and 3′UTR may also facilitate packaging of SARS-CoV-2. In some embodiments, the 5′UTR region of the recombinant SARS-Cov-2 construct described herein comprises at least 100 (including for example at least about any of 120, 140, 160, 180, 200, 220, 240, or 260) nucleotides corresponding to positions 1-265 of SEQ ID NO: 1. In some embodiments, the 5′UTR region of the recombinant SARS-Cov-2 construct described herein comprises a variant of at least 100 (including for example at least about 120, 140, 160, 180, 200, 220, 240, or 260) nucleotides corresponding to positions 1-265 of SEQ ID NO:1. In some embodiments, the nucleotide sequence of the variant is at least about any of 30%, 35%, 40%, 45%, 50%, 55%, 60%, 65%, 70%, 75%, 80%, 85%, 90%, 95%, 96%, 97%, 98%, or 99% homologous to a nucleotide sequence having at least 100 (including for example at least about 120, 140, 160, 180, 200, 220, 240, or 260) nucleotides corresponding to positions 1-265 of SEQ ID NO:1). In some embodiments, the 5′UTR region of the recombinant SARS-CoV-2 construct comprises nucleotides corresponding to positions 1-265 of SEQ ID NO:1. In some embodiments, the 5′UTR region of the recombinant SARS-CoV-2 construct comprises a nucleotide sequence that is at least about any of 30%, 35%, 40%, 45%, 50%, 55%, 60%, 65%, 70%, 75%, 80%, 85%, 90%, 95%, 96%, 97%, 98%, or 99% homologous to a nucleotide sequence corresponding to positions 1-265 of SEQ ID NO:1.

Similarly, the SARS-CoV-2 can have a 3′ untranslated region (3′ UTR) corresponding to positions 29675-29903 of SEQ ID NO: 1, including the 3′UTR core sequence corresponding to positions 29675-29870 and a polyA sequence corresponding to positions 29781-29903. In positive strand RNA viruses, the 3′—UTR can play a role in viral RNA replication because the origin of the minus-strand RNA replication intermediate is at the 3-end of the genome. In some embodiments, the 3′UTR region of the recombinant SARS-Cov-2 construct described herein comprises at least 100 (including for example at least about any of 120, 140, 160, 180, 200, or 220) nucleotides corresponding to positions 29675-29870 (or 29675-29903) of SEQ ID NO:1. In some embodiments, the 3′UTR region of the recombinant SARS-Cov-2 construct described herein comprises a variant of at least 100 (including for example at least about 120, 140, 160, 180, 200, or 220) nucleotides corresponding to positions 29675-29870 (or 29675-29903) of SEQ ID NO:1. In some embodiments, the nucleotide sequence of the variant is at least about any of 30%, 35%, 40%, 45%, 50%, 55%, 60%, 65%, 70%, 75%, 80%, 85%, 90%, 95%, 96%, 97%, 98%, or 99% homologous to a nucleotide sequence having at least 100 (including for example at least about 120, 140, 160, 180, 200, 220, 240, or 260) nucleotides corresponding to positions 29675-29870 (or 29675-29903) of SEQ ID NO:1). In some embodiments, the 3′UTR region of the recombinant SARS-CoV-2 construct comprises nucleotides corresponding to positions 29675-29870 (or 29675-29903) of SEQ ID NO:1. In some embodiments, the 3′UTR region of the recombinant SARS-CoV-2 construct comprises a nucleotide sequence that is at least about any of 30%, 35%, 40%, 45%, 50%, 55%, 60%, 65%, 70%, 75%, 80%, 85%, 90%, 95%, 96%, 97%, 98%, or 99% homologous to a nucleotide sequence corresponding to positions 29675-29870 (or 29675-29903) of SEQ ID NO:1.

The SARS-CoV-2 genome encodes four major structural proteins: the spike (S) protein, nucleocapsid (N) protein, membrane (M) protein, and the envelope (E) protein. Some of these proteins are part of a large polyprotein, which is at positions 266-21555 of SEQ ID NO: 1, where this open reading frame (ORF) is referred to as ORF1ab polyprotein and has SEQ ID NO: 2, shown below. In some embodiments, the recombinant SARS-CoV-2 construct does not comprises any portion of the ORF1ab nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF1ab nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF1ab nucleotide sequence, wherein the full length or a portion of the ORF1ab nucleotide sequence comprises a frameshift mutation, a deletion, an insertion, a non-sense mutation, or a missense mutation that renders no protein translation at all or no translation of a functional viral protein.

| | |
|---|---|
| 1 | MESLVPGFNE KTHVQLSLPV LQVRDVLVRG FGDSVEEVLS |
| 41 | EARQHLKDGT CGLVEVEKGV LPQLEQPYVF IKRSDARTAP |
| 81 | HGHVMVELVA ELEGIQYGRS GETLGVLVPH VGEIPVAYRK |
| 121 | VLLRKNGNKG AGGHSYGADL KSFDLGDELG TDPYEDFQEN |
| 161 | WNTKHSSGVT RELMRELNGG AYTRYVDNNF CGPDGYPLEC |
| 201 | IKDLLARAGK ASCTLSEQLD FIDTKRGVYC CREHEHEIAW |
| 241 | YTERSEKSYE LQTPFEIKLA KKFDTENGEC PNFVEPLNSI |
| 281 | IKTIQPRVEK KKLDGFMGRI RSVYPVASPN ECNQMCLSTL |
| 321 | MKCDHCGETS WQTGDFVKAT CEFCGTENLT KEGATTCGYL |
| 361 | PQNAVVKIYC PACHNSEVGP EHSLAEYHNE SGLKTILRKG |
| 401 | GRTIAFGGCV FSYVGCHNKC AYWVPRASAN IGCNHTGVVG |
| 441 | EGSEGLNDNL LEILQKEKVN INIVGDEKLN EEIAIILASE |
| 481 | SASTSAFVET VKGLDYKAFK QIVESCGNEK VTKGKAKKGA |
| 521 | WNIGEQKSIL SPLYAFASEA ARVVRSIFSR TLETAQNSVR |
| 561 | VLQKAAITIL DGISQYSLRL IDAMMFTSDL ATNNLVVMAY |
| 601 | ITGGVVQLTS QWLTNIFGTV YEKLKPVLDW LEEKFKEGVE |
| 641 | FLRDGWEIVK FISTCACEIV GGQIVTCAKE IKESVQTFFK |
| 681 | LVNKFLALCA DSIIIGGAKL KALNLGETFV THSKGLYRKC |
| 721 | VKSREETGLL MPLKAPKEII FLEGETLPTE VLTEEVVLKT |
| 761 | GDLQPLEQPT SEAVEAPLVG TPVCINGLML LEIKDTEKYC |
| 801 | ALAPNMMVTN NTFTLKGGAP TKVTFGDDTV IEVQGYKSVN |
| 841 | ITFELDERID KVLNEKCSAY TVELGTEVNE FACVVADAVI |
| 881 | KTLQPVSELL TPLGIDLDEW SMATYYLFDE SGEFKLASHM |
| 921 | YCSFYPPDED EEEGDCEEEE FEPSTQYEYG TEDDYQGKPL |
| 961 | EFGATSAALQ PEEEQEEDWL DDDSQQTVGQ QDGSEDNQTT |
| 1001 | TIQTIVEVQP QLEMELTPVV QTIEVNSFSG YLKLIDNVYI |
| 1041 | KNADIVEEAK KVKPTVVVNA ANVYLKHGGG VAGALNKATN |
| 1081 | NAMQVESDDY IATNGPLKVG GSCVLSGHNL AKHCLHVVGP |
| 1121 | NVNKGEDIQL LKSAYENFNQ HEVLLAPLLS AGIFGADPIH |
| 1161 | SLRVCVDTVR TNVYLAVEDK NLYDKLVSSF LEMKSEKQVE |
| 1201 | QKIAEIPKEE VKPFITESKP SVEQRKQDDK KIKACVEEVT |
| 1241 | TTLEETKELT ENLLLYIDIN GNLHPDSATL VSDIDITELK |
| 1281 | KDAPYIVGDV VQEGVLTAVV IPTKKAGGTT EMLAKALRKV |
| 1321 | PTDNYITTYP GQGLNGYTVE EAKTVLKKCK SAFYILPSII |
| 1361 | SNEKQEILGT VSWNLREMLA HAEETRKLMP VCVETKAIVS |
| 1401 | TIQRKYKGIK IQEGVVDYGA RFYFYTSKTT VASLINTLND |
| 1441 | LNETLVTMPL GYVTHGLNLE EAARYMRSLK VPATVSVSSP |
| 1481 | DAVTAYNGYL TSSSKTPEEH FIETISLAGS YKDWSYSGQS |
| 1521 | TQLGIEFLKR GDKSVYYTSN PTTFHLDGEV ITFDNLKTLL |
| 1561 | SLREVRTIKV FTTVDNINLH TQVVDMSMTY GQQFGPTYLD |
| 1601 | GADVTKIKPH NSHEGKTFYV LPNDDTLRVE AFEYYHTTDP |
| 1641 | SFLGRYMSAL NHTKKWKYPQ VNGLTSIKWA DNNCYLATAL |
| 1681 | LTLQQIELKF NPPALQDAYY RARAGEAANF CALILAYCNK |
| 1721 | TVGELGDVRE TMSYLFQHAN LDSCKRVLNV VCKTCGQQQT |
| 1761 | TLKGVEAVMY MGTLSYEQFK KGVQIPCTCG KQATKYLVQQ |
| 1801 | ESPFVMMSAP PAQYELKHGT FTCASEYTGN YQCGHYKHIT |
| 1841 | SKETLYCIDG ALLTKSSEYK GPITDVFYKE NSYTTTIKPV |
| 1881 | TYKLDGVVCT EIDPKLDNYY KKDNSYFTEQ PIDLVPNQPY |
| 1921 | PNASFDNFKF VCDNIKFADD LNQLTGYKKP ASRELKVTFF |
| 1961 | PDLNGDVVAI DYKHYTPSFK KGAKLLHKPI VWHVNNATNK |
| 2001 | ATYKPNTWCI RCLWSTKPVE TSNSFDVLKS EDAQGMDNLA |
| 2041 | CEDLKPVSEE VVENPTIQKD VLECNVKITE VVGDIILKPA |
| 2081 | NNSLKITEEV GHTDLMAAYV DNSSLTIKKP NELSRVLGLK |
| 2121 | TLATHGLAAV NSVPWDTIAN YAKPELNKVV STTTNIVTRC |
| 2161 | LNRVCTNYMP YFFTLLLQLC TFTRSTNSRI KASMPTTIAK |
| 2201 | NTVKSVGKFC LEASENYLKS PNFSKLINII IWFLLLSVCL |
| 2241 | GSLIYSTAAL GVLMSNLGMP SYCTGYREGY LNSTNVTIAT |
| 2281 | YCTGSIPCSV CLSGLDSLDT YPSLETIQIT ISSFKWDLTA |
| 2321 | FGLVAEWFLA YILFTRFFYV LGLAAIMQLF FSYFAVHFIS |
| 2361 | NSWLMWLIIN LVQMAPISAM VRMYIFFASF YYVWKSYVHV |
| 2401 | VDGCNSSTCM MCYKRNRATR VECTTIVNGV RRSFYVYANG |
| 2441 | GKGFCKLHNW NCVNCDTFCA GSTFISDEVA RDLSLQFKRP |
| 2481 | INPTDQSSYI VDSVTVKNGS IHLYFDKAGQ KTYERHSLSH |
| 2521 | FVNLDNLRAN NTKGSLPINV IVFDGKSKCE ESSAKSASVY |
| 2561 | YSQLMCQPIL LLDQALVSDV GDSAEVAVKM FDAYVNTESS |
| 2601 | TFNVPMEKLK TLVATAEAEL AKNVSLDNVL STFISAARQG |
| 2641 | FVDSDVETKD VVECLKLSHQ SDIEVTGDSC NNYMLTYNKV |
| 2481 | ENMTPRDLGA CIDCSARHIN AQVAKSHNIA LIWNVKDFMS |
| 2521 | LSEQLRKQIR SAAKKNNLPF KLTCATTRQV VNVVTTKIAL |
| 2561 | KGGKIVNNWL KQLIKVILVF LEVAAIFYLI TPVHVMSKHT |
| 2601 | DFSSEIIGYK AIDGGVTRDI ASTDTCFANK HADFDTWFSQ |
| 2641 | RGGSYTNDKA CPLIAAVITR EVGFVVPGLP GTILRTINGD |
| 2681 | FLHELPRVES AVGNICYTPS KLIEYTDFAT SACVLAAECT |
| 2721 | IFKDASGKPV PYCYDTNVLE GSVAYESLRP DTRYVLMDGS |
| 2761 | IIQFPNTYLE GSVRVVTTED SEYCRHGTCE RSEAGVCVST |
| 2801 | SGRWVLNNDY YRSLPGVFCG VDAVNLLTNM FTPLIQPIGA |
| 2841 | LDISASIVAG GIVAIVVTCL AYYEMRFRRA FGEYSHVVAF |
| 2881 | NTLLFLMSFT VLCLTPVYSF LPGVYSVIYL YLTFYLTNDV |
| 2921 | SFLAHIQWMV MFTPLVPFWI TIAYIICIST KHFYWFFSNY |
| 2961 | LKRRVVENGV SESTFEEAAL CTFLINKEMY LKLRSDVLLP |
| 3001 | LTQYNRYLAL YNKYKYFSGA MDTTSYREAA CCHLAKALND |
| 3041 | FSNSGSDVLY QPPQTSITSA VLQSGFRKMA FPSGKVEGCM |
| 3081 | VQVICGTTTL NGLWLDDVVY CPRHVICTSE DMLNPNYEDL |
| 3121 | LIRKSNHNFL VQAGNVQLRV IGHSMQNCVL KLKVDTANPK |
| 3161 | TPKYKFVRIQ PGQTFSVLAC YNGSPSGVYQ CAMRPNFTIK |
| 3201 | GSFLNGSCGS VGFNIDYDCV SFCYMHHMEL PTGVHAGTDL |
| 3241 | EGNFYGPFVD RQTAQAAGTD TTITVNVLAW LYAAVINGDR |
| 3281 | WFLNRFTTTL NDENLVAMKY NYEPLTQDHV DILGPLSAQT |
| 3321 | GIAVLDMCAS LKELLQNGMN GRTILGSALL EDEFTPFDVV |
| 3361 | RQCSGVTFQS AVKRTIKGTH HWLLLTILTS LLVLVQSTQW |
| 3401 | SLFFFLYENA FLPFAMGIIA MSAFAMMEVK HKHAFLCLFL |
| 3441 | LPSLATVAYF NMVYMPASWV MRIMTWLDMV DTSLSGFKLK |
| 3481 | DCVMYASAVV LLILMTARTV YDDGARRVWT LMNVLTLVYK |
| 3521 | VYYGNALDQA ISMWALIISV TSNYSGVVTT VMFLARGIVE |
| 3561 | MCVEYCPIFF ITGNTLQCIM LVYCFLGYFC TCYFGLFCLL |
| 3601 | NRYFRLTLGV YDYLVSTQEF RYMNSQGLLP PKNSIDAFKL |
| 3641 | NIKLLGVGGK PCIKVATVQS KMSDVKCTSV VLLSVLQQLR |
| 3681 | VESSSKLWAQ CVQLHNDILL AKDTTEAFEK MVSLLSVLLS |
| 3721 | MQGAVDINKL CEEMLDNRAT LQAIASEFSS LPSYAAFATA |
| 3761 | QEAYEQAVAN GDSEVVLKKL KKSLNVAKSE FDRDAAMQRK |
| 3801 | LEKMADQAMT QMYKQARSED KRAKVTSAMQ TMLFTMLRKL |
| 3841 | DNDALNNIIN NARDGCVPLN IIPLTTAAKL MVVIPDYNTY |
| 3881 | KNTCDGTTFT YASALWEIQQ VVDADSKIVQ LSEISMDNSP |
| 3921 | NLAWPLIVTA LRANSAVKLQ NNELSPVALR QMSCAAGTTQ |
| 3961 | TACTDDNALA YYNTTKGGRF VLALLSDLQD LKWARFPKSD |
| 4001 | GTGTIYTELE PPCRFVIDTP KGPKVKYLYF IKGLNNLNRG |
| 4041 | MVLGSLAATV RLQAGNATEV PANSTVLSFC AFAVDAAKAY |
| 4081 | KDYLASGGQP ITNCVKMLCT HTGTGQAITV TPEANMDQES |
| 4121 | FGGASCCLYC RCHIDHPNPK GFCDLKGKYV QIPTTCANDP |
| 4161 | VGFTLKNTVC TVCGMWKGYG CSCDQLREPM LQSADAQSEL |
| 4201 | NGFAV |

An RNA-dependent RNA polymerase is encoded at positions 13442-13468 and 13468-16236 of the SARS-CoV-2 SEQ ID NO: 1 nucleic acid. This RNA-dependent RNA polymerase has been assigned NCBI accession number YP_009725307 and has the following sequence (SEQ ID NO: 3). In some embodiments, the recombinant SARS-CoV-2 construct does not comprises any portion of the RNA-dependent RNA polymerase nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the RNA-dependent RNA polymerase nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the RNA-dependent RNA polymerase nucleotide sequence, wherein the full length or a portion of the RNA-dependent RNA polymerase nucleotide sequence comprises a frameshift mutation, a deletion, an insertion, a non-sense mutation, or a missense mutation that renders no protein translation at all or no translation of a functional viral protein.

| | |
|---|---|
| 1 | SADAQSFLNR VCGVSAARLT PCGTGTSTDV VYRAFDIYND |
| 41 | KVAGFAKFLK TNCCRFQEKD EDDNLIDSYF VVKRHTESNY |
| 81 | QHEETIYNLL KDCPAVAKHD FFKFRIDGDM VPHISRQRLT |
| 121 | KYTMADLVYA LRHFDEGNCD TLKEILVTYN CCDDDYENKK |
| 161 | DWYDFVENPD ILRVYANLGE RVRQALLKTV QFCDAMRNAG |
| 201 | IVGVLTLDNQ DLNGNWYDFG DFIQTTPGSG VPVVDSYYSL |
| 241 | LMPILTLTRA LTAESHVDTD LTKPYIKWDL LKYDFTEERL |
| 281 | KLFDRYFKYW DQTYHPNCVN CLDDRCILHC ANFNVLESTV |
| 321 | FPPTSFGPLV RKIFVDGVPF VVSTGYHFRE LGVVHNQDVN |
| 361 | LHSSRLSFKE LLVYAADPAM HAASGNLLLD KRTTCESVAA |
| 401 | LINNVAFQTV KPGNENKDFY DFAVSKGFFK EGSSVELKHE |
| 441 | FFAQDGNAAI SDYDYYRYNL PTMCDIRQLL FVVEVVDKYF |
| 481 | DCYDGGCINA NQVIVNNLDK SAGEPENKWG KARLYYDSMS |
| 521 | YEDQDALFAY TKRNVIPTIT QMNLKYAISA KNRARTVAGV |
| 561 | SICSTMINRQ FHQKLLKSIA ATRGATVVIG TSKFYGGWHN |
| 601 | MLKTVYSDVE NPHLMGWDYP KCDRAMPNML RIMASLVLAR |
| 641 | KHTTCCSLSH RFYRLANECA QVLSEMVMCG GSLYVKPGGT |
| 681 | SSGDATTAYA NSVFNICQAV TANVNALLST DGNKIADKYV |
| 721 | RNLQHRLYEC LYRNRDVDTD FVNEFYAYLR KHFSMMILSD |
| 761 | DAVVCFNSTY ASQGLVASIK NFKSVLYYQN NVEMSEAKCW |
| 801 | TETDLTKGPH EFCSQHTMLV KQGDDYVYLP YPDPSRILGA |
| 841 | GCFVDDIVKT DGTLMIERFV SLAIDAYPLT KHPNQEYADV |
| 881 | FHLYLQYIRK LHDELTGHML DMYSVMLIND NTSRYWEPEF |
| 921 | YEAMYTPHTV LQ |

A helicase is encoded at positions 16237-18039 of the SARS-CoV-2 SEQ ID NO: 1 nucleic acid. This helicase has been assigned NCBI accession number YP_009725308.1 and has the following sequence (SEQ ID NO: 4, shown below). In some embodiments, the recombinant SARS-CoV-2 construct does not comprises any portion of the helicase nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the helicase nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the helicase nucleotide sequence, wherein the full length or a portion of the helicase nucleotide sequence comprises a frameshift mutation, a deletion, an insertion, a non-sense mutation, or a missense mutation that renders no protein translation at all or no translation of a functional viral protein.

| | |
|---|---|
| 1 | AVGACVLCNS QTSLRCGACI RRPFLCCKCC YDHVISTSHK |
| 41 | LVLSVNPYVC NAPGCDVIDV TQLYLGGMSY YCKSHKPPIS |
| 81 | FPLCANGQVF GLYKNTCVGS DNVTDFNAIA TCDWTNAGDY |
| 121 | ILANTCTERL KLFAAETLKA TEETFKLSYG IATVREVLSD |
| 161 | RELHLSWEVG KPRPPLNRNY VFTGYRVTKN SKVQIGEYTF |
| 201 | EKGDYGDAVV YRGTTTYKLN VGDYFVLTSH TVMPLSAPTL |
| 241 | VPQEHYVRIT GLYPTLNISD EFSSNVANYQ KVGMQKYSTL |
| 281 | QGPPGTGKSH FAIGLALYYP SARIVYTACS HAAVDALCEK |
| 321 | ALKYLPIDKC SRIIPARARV ECFDKEKVNS TLEQYVFCTV |
| 361 | NALPETTADI VVEDEISMAT NYDLSVVNAR LRAKHYVYIG |
| 401 | DPAQLPAPRT LLTKGTLEPE YENSVCRLMK TIGPDMELGT |
| 441 | CRRCPAEIVD TVSALVYDNK LKAHKDKSAQ CFKMFYKGVI |
| 481 | THDVSSAINR PQIGVVREFL TRNPAWRKAV FISPYNSQNA |
| 521 | VASKILGLPT QTVDSSQGSE YDYVIFTQTT ETAHSCNVNR |
| 561 | FNVAITRAKV GILCIMSDRD LYDKLQFTSL EIPRRNVATL |
| 601 | Q |

The SARS-CoV-2 can have an ORF at positions 21563-25384 (gene S) of the SEQ ID NO: 1 sequence that can be referred to as GU280_gp02, where this ORF encodes a surface glycoprotein or a spike glycoprotein (SEQ ID NO: 5, shown below). In some embodiments, the recombinant SARS-CoV-2 construct does not comprises any portion of the gene S nucleotide sequence. In some embodiments, the recombinant SARS-CoV-2 construct comprises a portion (e.g., no more than any of 90%, 80%, 70%, 60%, 50%, 40%, 30%, 20%, 100%, or 5%) of the gene S nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the gene S nucleotide sequence, wherein the full length or a portion of the gene S nucleotide sequence comprises a frameshift mutation, a deletion, an insertion, a non-sense mutation, or a missense mutation that renders no protein translation at all or no translation of a functional viral protein.

| | |
|---|---|
| 1 | MFVFLVLLPL VSSQCVNLTT RTQLPPAYTN SFTRGVYYPD |
| 41 | KVFRSSVLHS TQDLFLPFFS NVTWFHAIHV SGTNGTKRED |
| 81 | NPVLPENDGV YFASTEKSNI IRGWIFGTTL DSKTQSLLIV |
| 121 | NNATNVVIKV CEFQFCNDPF LGVYYHKNNK SWMESEFRVY |
| 161 | SSANNCTFEY VSQPFLMDLE GKQGNEKNLR EFVFKNIDGY |
| 201 | FKIYSKHTPI NLVRDLPQGF SALEPLVDLP IGINITRFQT |
| 241 | LLALHRSYLT PGDSSSGWTA GAAAYYVGYL QPRTFLLKYN |
| 281 | ENGTITDAVD CALDPLSETK CTLKSFTVEK GIYQTSNERV |
| 321 | QPTESIVRFP NITNLCPFGE VENATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLERKSN LKPFERDIST EIYQAGSTPC |
| 481 | NGVEGENCYF PLQSYGFQPT NGVGYQPYRV VVLSFELLHA |
| 521 | PATVCGPKKS TNLVKNKCVN FNENGLTGTG VLTESNKKEL |
| 561 | PFQQFGRDIA DTTDAVRDPQ TLEILDITPC SFGGVSVITP |
| 601 | GTNTSNQVAV LYQDVNCTEV PVAIHADQLT PTWRVYSTGS |
| 641 | NVFQTRAGCL IGAEHVNNSY ECDIPIGAGI CASYQTQTNS |
| 681 | PRRARSVASQ SIIAYTMSLG AENSVAYSNN SIAIPTNETI |
| 721 | SVTTEILPVS MTKTSVDCTM YICGDSTECS NLLLQYGSFC |
| 761 | TQLNRALTGI AVEQDKNTQE VFAQVKQIYK TPPIKDEGGF |
| 801 | NFSQILPDPS KPSKRSFIED LLENKVTLAD AGFIKQYGDC |
| 841 | LGDIAARDLI CAQKENGLTV LPPLLTDEMI AQYTSALLAG |
| 881 | TITSGWTFGA GAALQIPFAM QMAYRENGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL HYT |

The S or spike protein is responsible for facilitating entry of the SARS-CoV-2 into cells. It is composed of a short intracellular tail, a transmembrane anchor, and a large ectodomain that consists of a receptor binding Si subunit and a membrane-fusing S2 subunit. The spike receptor binding domain (“RBD domain”) can reside at amino acid positions 330-583 of the SEQ ID NO: 5 spike protein of SARS-CoV-2 (SEQ ID NO: 6, shown below). In some embodiments, the recombinant SARS-CoV-2 construct does not comprises any portion of the S protein nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the S protein nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the S protein nucleotide sequence, wherein the full length or a portion of the S protein nucleotide sequence comprises a frameshift mutation, a deletion, an insertion, a non-sense mutation, or a missense mutation that renders no protein translation at all or no translation of a functional viral protein. In some embodiments, the recombinant SARS-CoV-2 construct does not comprises any portion of the RBD domain nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the RBD domain nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the RBD domain nucleotide sequence, wherein the full length or a portion of the RBD domain nucleotide sequence comprises a frameshift mutation, a deletion, an insertion, a non-sense mutation, or a missense mutation that renders no protein translation at all or no translation of a functional viral protein domain.

| | |
|---|---|
| 330 | P NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLERKSN LKPFERDIST EIYQAGSTPC |
| 481 | NGVEGENCYF PLQSYGFQPT NGVGYQPYRV VVLSFELLHA |
| 521 | PATVCGPKKS TNLVKNKCVN FNENGLTGTG VLTESNKKEL |
| 561 | PFQQFGRDIA DTTDAVRDPQ TLE |

Analysis of this receptor binding motif (RBM) in the spike protein showed that most of the amino acid residues essential for receptor binding were conserved between SARS-CoV and SARS-CoV-2, suggesting that both CoV strains use the same host receptor for entry into host cells. The entry receptor utilized by SARS-CoV is the angiotensin-converting enzyme 2 (ACE-2).

The SARS-CoV-2 spike protein membrane-fusing S2 domain can be at positions 662-1270 of the SEQ ID NO: 5 spike protein of SARS-CoV-2 (SEQ ID NO: 7, shown below). In some embodiments, the recombinant SARS-CoV-2 construct does not comprises any portion of the S2 domain nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the S2 domain nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the S2 domain nucleotide sequence, wherein the full length or a portion of the S2 domain nucleotide sequence comprises a frameshift mutation, a deletion, an insertion, a non-sense mutation, or a missense mutation that renders no protein translation at all or no translation of a functional viral protein domain.

| | |
|---|---|
| 662 | CDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLENKVTLAD AGFIKQYGDC |
| 841 | LGDIAARDLI CAQKENGLTV LPPLLTDEMI AQYTSALLAG |
| 881 | TITSGWTFGA GAALQIPFAM QMAYRENGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL H |

The SARS-CoV-2 can have an ORF at positions 2720-8554 of the SEQ ID NO: 1 sequence that can be referred to as nsp3, which includes transmembrane domain 1 (TM1). This nsp3 ORF with transmembrane domain 1 has NCBI accession no. YP_009725299.1, and is shown below as SEQ ID NO: 8. In some embodiments, the recombinant SARS-CoV-2 construct does not comprises any portion of the nsp3 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the nsp3 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the nsp3 nucleotide sequence, wherein the full length or a portion of the nsp3 nucleotide sequence comprises a frameshift mutation, a deletion, an insertion, a non-sense mutation, or a missense mutation that renders no protein translation at all or no translation of a functional viral protein.

| | |
|---|---|
| 1 | APTKVTFGDD TVIEVQGYKS VNITFELDER IDKVLNEKCS |
| 41 | AYTVELGTEV NEFACVVADA VIKTLQPVSE LLTPLGIDLD |
| 81 | EWSMATYYLF DESGEFKLAS HMYCSFYPPD EDEEEGDCEE |
| 121 | EEFEPSTQYE YGTEDDYQGK PLEFGATSAA LQPEEEQEED |
| 161 | WLDDDSQQTV GQQDGSEDNQ TTTIQTIVEV QPQLEMELTP |
| 201 | VVQTIEVNSF SGYLKLIDNV YIKNADIVEE AKKVKPTVVV |
| 241 | NAANVYLKHG GGVAGALNKA TNNAMQVESD DYIATNGPLK |
| 281 | VGGSCVLSGH NLAKHCLHVV GPNVNKGEDI QLLKSAYENF |
| 321 | NQHEVLLAPL LSAGIFGADP IHSLRVCVDT VRINVYLAVE |
| 361 | DKNLYDKLVS SFLEMKSEKQ VEQKIAEIPK EEVKPFITES |
| 401 | KPSVEQRKQD DKKIKACVEE VTTTLEETKF LTENLLLYID |
| 441 | INGNLHPDSA TLVSDIDITF LKKDAPYIVG DVVQEGVLTA |
| 481 | VVIPTKKAGG TTEMLAKALR KVPTDNYITT YPGQGLNGYT |
| 521 | VEEAKTVLKK CKSAFYILPS IISNEKQEIL GTVSWNLREM |
| 561 | LAHAEETRKL MPVCVETKAI VSTIQRKYKG IKIQEGVVDY |
| 601 | GARFYFYTSK TIVASLINTL NDLNETLVTM PLGYVTHGLN |
| 641 | LEEAARYMRS LKVPATVSVS SPDAVTAYNG YLTSSSKTPE |
| 681 | EHFIETISLA GSYKDWSYSG QSTQLGIEFL KRGDKSVYYT |
| 721 | SNPTTFHLDG EVITEDNLKT LLSLREVRTI KVFTTVDNIN |
| 761 | LHTQVVDMSM TYGQQFGPTY LDGADVTKIK PHNSHEGKTF |
| 801 | YVLPNDDTLR VEAFEYYHTT DPSFLGRYMS ALNHTKKWKY |
| 841 | PQVNGLTSIK WADNNCYLAT ALLTLQQIEL KENPPALQDA |
| 881 | YYRARAGEAA NFCALILAYC NKTVGELGDV RETMSYLFQH |
| 921 | ANLDSCKRVL NVVCKTCGQQ QTTLKGVEAV MYMGTLSYEQ |
| 961 | FKKGVQIPCT CGKQATKYLV QQESPFVMMS APPAQYELKH |
| 1001 | GTFTCASEYT GNYQCGHYKH ITSKETLYCI DGALLTKSSE |
| 1041 | YKGPITDVFY KENSYTTTIK PVTYKLDGVV CTEIDPKLDN |
| 1081 | YYKKDNSYFT EQPIDLVPNQ PYPNASFDNF KFVCDNIKFA |
| 1121 | DDLNQLTGYK KPASRELKVT FFPDLNGDVV AIDYKHYTPS |
| 1161 | FKKGAKLLHK PIVWHVNNAT NKATYKPNTW CIRCLWSTKP |
| 1201 | VETSNSFDVL KSEDAQGMDN LACEDLKPVS EEVVENPTIQ |
| 1241 | KDVLECNVKT TEVVGDIILK PANNSLKITE EVGHTDLMAA |
| 1281 | YVDNSSLTIK KPNELSRVLG LKTLATHGLA AVNSVPWDTI |
| 1321 | ANYAKPFLNK VVSTTTNIVT RCLNRVCTNY MPYFFTLLLQ |
| 1361 | LCTFTRSTNS RIKASMPTTI AKNTVKSVGK FCLEASFNYL |
| 1401 | KSPNFSKLIN IIIWFLLLSV CLGSLIYSTA ALGVLMSNLG |
| 1441 | MPSYCTGYRE GYLNSTNVTI ATYCTGSIPC SVCLSGLDSL |
| 1481 | DTYPSLETIQ ITISSFKWDL TAFGLVAEWF LAYILFTRFF |
| 1521 | YVLGLAAIMQ LFFSYFAVHF ISNSWLMWLI INLVQMAPIS |
| 1561 | AMVRMYIFFA SFYYVWKSYV HVVDGCNSST CMMCYKRNRA |
| 1601 | TRVECTTIVN GVRRSFYVYA NGGKGFCKLH NWNCVNCDTE |
| 1641 | CAGSTFISDE VARDLSLQFK RPINPTDQSS YIVDSVTVKN |
| 1681 | GSIHLYFDKA GQKTYERHSL SHFVNLDNLR ANNTKGSLPI |
| 1721 | NVIVEDGKSK CEESSAKSAS VYYSQLMCQP ILLLDQALVS |
| 1761 | DVGDSAEVAV KMFDAYVNTF SSTFNVPMEK LKTLVATAEA |
| 1801 | ELAKNVSLDN VLSTFISAAR QGFVDSDVET KDVVECLKLS |
| 1841 | HQSDIEVTGD SCNNYMLTYN KVENMTPRDL GACIDCSARH |
| 1881 | INAQVAKSHN IALIWNVKDF MSLSEQLRKQ IRSAAKKNNL |
| 1921 | PFKLTCATTR QVVNVVTTKI ALKGG |

The nsp3 protein has additional conserved domains including an N-terminal acidic (Ac), a predicted phosphoesterase, a papain-like proteinase, Y-domain, transmembrane domain 1 (TM1), and an adenosine diphosphate-ribose 1″-phosphatase (ADRP).

The SARS-CoV-2 can have an ORF at positions 8555-10054 of the SEQ ID NO:1 sequence that can be referred to as nsp4B_TM, which includes transmembrane domain 2 (TM2). This nsp4B_TM ORF with transmembrane domain 2 has NCBI accession no. YP_009725300, and is shown below as SEQ ID NO: 9. In some embodiments, the recombinant SARS-CoV-2 construct does not comprises any portion of the nsp4B_TM nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the nsp4B_TM nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the nsp4B_TM nucleotide sequence, wherein the full length or a portion of the nsp4B_TM nucleotide sequence comprises a frameshift mutation, a deletion, an insertion, a non-sense mutation, or a missense mutation that renders no protein translation at all or no translation of a functional viral protein.

| | |
|---|---|
| 1 | KIVNNWLKQL IKVTLVFLFV AAIFYLITPV HVMSKHTDFS |
| 41 | SEIIGYKAID GGVTRDIAST DTCFANKHAD FDTWFSQRGG |
| 81 | SYTNDKACPL IAAVITREVG FVVPGLPGTI LRTINGDELH |
| 121 | FLPRVFSAVG NICYTPSKLI EYTDFATSAC VLAAECTIFK |
| 161 | DASGKPVPYC YDTNVLEGSV AYESLRPDTR YVLMDGSIIQ |
| 201 | FPNTYLEGSV RVVTTEDSEY CRHGTCERSE AGVCVSTSGR |
| 241 | WVLNNDYYRS LPGVFCGVDA VNLLTNMFTP LIQPIGALDI |
| 281 | SASIVAGGIV AIVVTCLAYY FMRFRRAFGE YSHVVAFNTL |
| 321 | LFLMSFTVLC LTPVYSFLPG VYSVIYLYLT FYLTNDVSFL |
| 361 | AHIQWMVMFT PLVPFWITIA YIICISTKHF YWFFSNYLKR |
| 401 | RVVENGVSES TFEEAALCTF LLNKEMYLKL RSDVLLPLTQ |
| 441 | YNRYLALYNK YKYFSGAMDT TSYREAACCH LAKALNDFSN |
| 481 | SGSDVLYQPP QTSITSAVLQ |

The SARS-CoV-2 can have an ORF at positions 25393-26220 (ORF3a) of the SEQ ID NO: 1 sequence that can be referred to as GU280_gp03 (SEQ ID NO: 10, shown below). In some embodiments, the recombinant SARS-CoV-2 construct does not comprises any portion of the ORF3a nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF3a nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF3a nucleotide sequence, wherein the full length or a portion of the ORF3a nucleotide sequence comprises a frameshift mutation, a deletion, an insertion, a non-sense mutation, or a missense mutation that renders no protein translation at all or no translation of a functional viral protein.

| | |
|---|---|
| 1 | MDLFMRIFTI GTVTLKQGEI KDATPSDEVR ATATIPIQAS |
| 41 | LPFGWLIVGV ALLAVFQSAS KIITLKKRWQ LALSKGVHFV |
| 81 | CNLLLLFVTV YSHLLLVAAG LEAPFLYLYA LVYFLQSINE |
| 121 | VRIIMRLWLC WKCRSKNPLL YDANYFLCWH TNCYDYCIPY |
| 161 | NSVISSIVIT SGDGTTSPIS EHDYQIGGYT EKWESGVKDC |
| 201 | VVLHSYFTSD YYQLYSTQLS TDTGVEHVTF FIYNKIVDEP |
| 241 | EEHVQIHTID GSSGVVNPVM EPIYDEPTTT TSVPL |

The SARS-CoV-2 can have an ORF at positions 26245-26472 (gene E) of the SEQ ID NO: 1 sequence that can be referred to as GU280_gp04 (SEQ ID NO: 11, shown below).

| | |
|---|---|
| 1 | MYSFVSEETG TLIVNSVLLF LAFVVFLLVT LAILTALRLC |
| 41 | AYCCNIVNVS LVKPSFYVYS RVKNLNSSRV PDLLV |

The SEQ ID NO: 11 protein is a structural protein, for example, an envelope protein. In some embodiments, the recombinant SARS-CoV-2 construct does not comprises any portion of the gene E nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the gene E nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the gene E nucleotide sequence, wherein the full length or a portion of the gene E nucleotide sequence comprises a frameshift mutation, a deletion, an insertion, a non-sense mutation, or a missense mutation that renders no protein translation at all or no translation of a functional viral protein.

The SARS-CoV-2 can have an ORF at positions 27202-27191 (M protein gene; ORF5) of the SEQ ID NO: 1 sequence that can be referred to as GU280_gp05 (SEQ ID NO: 12, shown below).

| | |
|---|---|
| 1 | MADSNGTITV EELKKLLEQW NLVIGFLFLT WICLLQFAYA |
| 41 | NRNRFLYIIK LIFLWLLWPV TLACEVLAAV YRINWITGGI |
| 121 | AIAMACLVGL MWLSYFIASF RLFARTRSMW SENPETNILL |
| 161 | NVPLHGTILT RPLLESELVI GAVILRGHLR IAGHHLGRCD |
| 201 | IKDLPKEITV ATSRTLSYYK LGASQRVAGD SGFAAYSRYR |
| 241 | IGNYKLNTDH SSSSDNIA |
| 121 | LLVQ |

The SEQ ID NO: 12 protein is a structural protein, for example, a membrane PGP-32.DN glycoprotein. In some embodiments, the recombinant SARS-CoV-2 construct does not comprises any portion of the ORF5 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF5 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF5 nucleotide sequence, wherein the full length or a portion of the ORF5 nucleotide sequence comprises a frameshift mutation, a deletion, an insertion, a non-sense mutation, or a missense mutation that renders no protein translation at all or no translation of a functional viral protein. The SARS-CoV-2 can have an ORF at positions 27202-27387 (ORF6) of the SEQ ID NO: 1 sequence that can be referred to as GU280_gp06 (SEQ ID NO: 13, shown below). In some embodiments, the recombinant SARS-CoV-2 construct does not comprises any portion of the ORF6 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF6 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF6 nucleotide sequence, wherein the full length or a portion of the ORF6 nucleotide sequence comprises a frameshift mutation, a deletion, an insertion, a non-sense mutation, or a missense mutation that renders no protein translation at all or no translation of a functional viral protein.

| | |
|---|---|
| 1 | MFHLVDFQVT IAEILLIIMR TFKVSIWNLD YIINLIIKNL |
| 41 | SKSLTENKYS QLDEEQPMEI D |

The SARS-CoV-2 can have an ORF at positions 27394-27759 (ORF7a) of the SEQ ID NO: 1 sequence that can be referred to as GU280_gp07 (SEQ ID NO: 14, shown below). In some embodiments, the recombinant SARS-CoV-2 construct does not comprises any portion of the ORF7a nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF7a nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF7a nucleotide sequence, wherein the full length or a portion of the ORF7a nucleotide sequence comprises a frameshift mutation, a deletion, an insertion, a non-sense mutation, or a missense mutation that renders no protein translation at all or no translation of a functional viral protein.

| | |
|---|---|
| 1 | MKIILFLALI TLATCELYHY QECVRGTTVL LKEPCSSGTY |
| 41 | EGNSPFHPLA DNKFALTCES TQFAFACPDG VKHVYQLRAR |
| 121 | SVSPKLFIRQ EEVQELYSPI FLIVAAIVFI TLCFTLKRKT |
| 161 | E |

The SARS-CoV-2 can have an ORF at positions 27756-27887 (ORF7b) of the SEQ ID NO: 1 sequence that can be referred to as GU280_gp08 (SEQ ID NO: 15, shown below). In some embodiments, the recombinant SARS-CoV-2 construct does not comprises any portion of the ORF7b nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF7b nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF7b nucleotide sequence, wherein the full length or a portion of the ORF7b nucleotide sequence comprises a frameshift mutation, a deletion, an insertion, a non-sense mutation, or a missense mutation that renders no protein translation at all or no translation of a functional viral protein.

| | |
|---|---|
| 1 | MIELSLIDFY LCFLAFLLFL VLIMLIIFWE SLELQDHNET |
| 41 | CHA |

The SARS-CoV-2 can have an ORF at positions 27894-28259 (ORF8) of the SEQ ID NO: 1 sequence that can be referred to as GU280_gp09 (SEQ ID NO: 16, shown below). In some embodiments, the recombinant SARS-CoV-2 construct does not comprises any portion of the ORF8 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF8 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF8 nucleotide sequence, wherein the full length or a portion of the ORF8 nucleotide sequence comprises a frameshift mutation, a deletion, an insertion, a non-sense mutation, or a missense mutation that renders no protein translation at all or no translation of a functional viral protein.

| | |
|---|---|
| 1 | MKFLVFLGII TTVAAFHQEC SLQSCTQHQP YVVDDPCPIH |
| 41 | FYSKWYIRVG ARKSAPLIEL CVDEAGSKSP IQYIDIGNYT |
| 121 | VSCLPFTINC QEPKLGSLVV RCSFYEDFLE YHDVRVVLDE |
| 161 | I |

The SARS-CoV-2 can have an ORF at positions 28274-29533 (gene N; ORF9) of the SEQ ID NO: 1 sequence that can be referred to as GU280_gp10 (SEQ ID NO: 17, shown below).

| | |
|---|---|
| 1 | MSDNGPQNQR NAPRITFGGP SDSTGSNQNG ERSGARSKQR |
| 41 | RPQGLPNNTA SWFTALTQHG KEDLKFPRGQ GVPINTNSSP |
| 121 | DDQIGYYRRA TRRIRGGDGK MKDLSPRWYF YYLGTGPEAG |
| 161 | LPYGANKDGI IWVATEGALN TPKDHIGTRN PANNAAIVLQ |
| 201 | LPQGTTLPKG FYAEGSRGGS QASSRSSSRS RNSSRNSTPG |
| 241 | SSRGTSPARM AGNGGDAALA LLLLDRLNQL ESKMSGKGQQ |
| 281 | QQGQTVTKKS AAEASKKPRQ KRTATKAYNV TQAFGRRGPE |
| 521 | QTQGNFGDQE LIRQGTDYKH WPQIAQFAPS ASAFFGMSRI |
| 561 | GMEVTPSGTW LTYTGAIKLD DKDPNFKDQV ILLNKHIDAY |
| 601 | KTFPPTEPKK DKKKKADETQ ALPQRQKKQQ TVTLLPAADL |
| 641 | DDFSKQLQQS MSSADSTQA |

The SEQ ID NO: 17 protein is a structural protein, for example, a nucleocapsid phosphoprotein. In some embodiments, the recombinant SARS-CoV-2 construct does not comprises any portion of the ORF9 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF9 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF9 nucleotide sequence, wherein the full length or a portion of the ORF9 nucleotide sequence comprises a frameshift mutation, a deletion, an insertion, a non-sense mutation, or a missense mutation that renders no protein translation at all or no translation of a functional viral protein.

The SARS-CoV-2 can have an ORF at positions 29558-29674 (ORF10) of the SEQ ID NO: 1 sequence that can be referred to as GU280_gp11 (SEQ ID NO: 19, shown below). In some embodiments, the recombinant SARS-CoV-2 construct does not comprises any portion of the ORF10 nucleotide sequence. In some embodiments, the recombinant SARS-CoV-2 construct comprises a portion (e.g., no more than any of 90%, 80%, 70%, 60%, 50%, 40%, 30%, 20%, 10%, or 5%) of the ORF10 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the ORF10 nucleotide sequence, wherein the full length or a portion of the ORF10 nucleotide sequence comprises a frameshift mutation, a deletion, an insertion, a non-sense mutation, or a missense mutation that renders no protein translation at all or no translation of a functional viral protein.

| | |
|---|---|
| 1 | MGYINVFAFP FTIYSLLLCR MNSRNYIAQV DVVNFNLT |

The SARS-CoV-2 can have a stem-loops at positions 29609-29644 and 29629-29657, of SEQ ID NO: 1, which is within the encoded GU280_gp11. For example, the SARS-CoV-2 stem-loop at positions 29609-29644 of SEQ ID NO: 1, is shown below as SEQ ID NO: 20. In some embodiments, the recombinant SARS-CoV-2 construct comprises SEQ ID NO: 20, or a sequence comprising at least about 90% sequence identity (such as at least any of about 91%, 92%, 93%, 94%, 95%, 96%, 97%, 98%, or 99% sequence identity) to the sequence of SEQ ID NO: 20. In some embodiments, the recombinant SARS-CoV-2 construct does not comprise SEQ ID NO:20.

| | |
|---|---|
| 29601 | TT GTGCAGAATG AATTCTCGTA ACTACATAGC |
| 29641 | ACAA |

For example, the SARS-CoV-2 stem-loop at positions 29629-29657 of SEQ ID NO: 1 is shown below as SEQ ID NO: 21. In some embodiments, the recombinant SARS-CoV-2 construct comprises SEQ ID NO: 21, or a sequence comprising at least about 90% sequence identity (such as at least any of about 91%, 92%, 93%, 94%, 95%, 96%, 97%, 98%, or 99% sequence identity) to the sequence of SEQ ID NO: 21. In some embodiments, the recombinant SARS-CoV-2 construct does not comprise SEQ ID NO:21.

| | |
|---|---|
| 29629 | ta actacatagc acaagtagat gtagtta |

The SARS-CoV-2 can have an ORF at positions 12686-13024 (nsp9) of the SEQ ID NO: 1 sequence that encodes a ssRNA-binding protein with NCBI accession number YP_009725305.1, which has the following sequence (SEQ ID NO: 22). In some embodiments, the recombinant SARS-CoV-2 construct does not comprises any portion of the nsp9 nucleotide sequence. In some embodiments, the recombinant SARS-CoV-2 construct comprises a portion (e.g., no more than any of 90%, 80% , 70%, 60%, 50%, 40%, 30%, 20%, 10%, or 5%) of the nsp9 nucleotide sequence. In some embodiments, the recombinant SARS-CoV-2 construct comprises a full length or a portion (e.g., at least about any of 90%, 80%, 70%, 60%, 50%, 40%, 30%, 20%, 10%, or 5%) of the nsp9 nucleotide sequence, wherein the full length or a portion of the nsp9 nucleotide sequence comprises a frameshift mutation, a deletion, an insertion, a non-sense mutation, or a missense mutation that renders no protein translation at all or no translation of a functional viral protein.

| | |
|---|---|
| 1 | NNELSPVALR QMSCAAGTTQ TACTDDNALA YYNTTKGGRE |
| 41 | VLALLSDLQD LKWARFPKSD GTGTIYTELE PPCRFVTDTP |
| 81 | KGPKVKYLYF IKGLNNLNRG MVLGSLAATV RLQ |

The foregoing nucleotide sequences are DNA sequences. In some embodiments, the SARS-CoV-2 nucleic acids used in the recombinant SARS-CoV-2 constructs described herein are DNA sequences. In some embodiments, the SARS-CoV-2 nucleic acids used in the recombinant SARS-CoV-2 constructs described herein are RNA sequences. In some embodiments, the recombinant SARS-CoV-2 constructs described herein comprise both DNA and RNA sequences (e.g., SARS-CoV-2 DNA sequences and SARS-CoV-2 RNA sequences). It is to be understood that, when the SARS-CoV-2 construct is RNA, the nucleotide sequence of the construct would be the RNA sequence corresponding to the DNA sequences provided herein.

In addition, the SARS-CoV-2 genome can naturally have structural variations that are reflections of sequence variations. Hence, the SARS-CoV-2 used in the recombinant SARS-CoV-2 constructs described herein can, for example, can have one or more nucleotide or amino acid differences from the sequences shown above. In some cases, the SARS-CoV-2 nucleic acids used in the recombinant SARS-CoV-2 constructs described herein can, for example, have two, three, four, five, six, seven, eight, nine, ten, fifteen, twenty, twenty-five, thirty, or more nucleotide or amino acid differences from the sequences shown above. In some embodiments, the recombinant SARS-CoV-2 construct can comprises a sequence that is at least about any of at least about any of 30%, 35%, 40%, 45%, 50%, 55%, 60%, 65%, 70%, 75%, 80%, 85%, 90%, 95%, 96%, 97%, 98%, or 99% homologous to a nucleotide sequence discussed above for ORF1ab, RNA-dependent RNA polymerase, helicase, gene S, S protein, RBD domain, S2 domain, nsp3, nsp4B, ORF3a, gene E, ORF5, ORF6, ORF7a, ORF7b, ORF8, ORF9, ORF10, SEQ ID NO:20, SEQ ID NO:21, or a portion thereof.

The recombinant SARS-CoV-2 constructs herein can have portions of the SARS-CoV-2 genome, where the deletions of the genome include at least 100, at least 500, at least 1000, at least 1500, at least 2000, at least 2500, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, at least 20,000, at least 21,000, at least 22,000, at least 23,000, at least 24,000, at least 25,000, at least 26,000, at least 27,000, at least 27500, or at least 28000 nucleotides of the SARS-CoV-2 genome.

A recombinant SARS-CoV-2 construct of the present disclosure comprises a 5′UTR region of a SARS-Cov-2 5′UTR or a variant thereof, an optional intervening sequence, and a 3′UTR region of a SARS-Cov-2 3′UTR or a variant thereof. In some embodiments, the total length of the 5′UTR region, the optional intervening sequence, and the 3′UTR region in the recombinant SARS-CoV-2 construct is about 1,000 to about 30,000 bp, such as between any of about 1,000 to about 20,000, about 1,000 and about 10,000 bp, about 1,000 and about 5,000 bp, about 2,000 and about 3,500 bp, about 5,000 and about 15,000 bp, about 10,000 and about 20,000 bp, about 15,000 and about 25,000 bp, about 20,000 and about 30,000 bp. In some embodiments, the total length of the 5′UTR region, the optional intervening sequence, and the 3′UTR region in the recombinant SARS-CoV-2 construct is greater than about 1,000 bp, such as greater than any of about 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500 bp, 30,000 bp, or more. In some embodiments, the total length of the 5′UTR region, the optional intervening sequence, and the 3′UTR region in the recombinant SARS-CoV-2 construct is less than about 30,000 bp, such as less than any of about 29,000, 28,500, 28,000, 27,500, 27,000, 26,500, 26,000, 25,500, 25,000, 24,500, 24,000, 23,500, 23,000, 22,500, 22,000, 21,500, 21,000, 19,500, 19,000, 18,500, 18,000, 17,500, 17,000, 16,500, 16,000, 15,500, 15,000, 14,500, 14,000, 13,500, 13,000, 12,500, 12,000, 11,500, 11,000, 10,500, 10,000, 9,500, 9,000, 8,500, 8,000, 7,500, 7,000, 6,500, 6,000, 5,500, 5,000, 4,500, 4,000, 3,500, 3,000, 2,500, 2,000, 1,500, 1,000 bp, or fewer in total length. In some embodiments, the total length of the 5′UTR region, the optional intervening sequence, and the 3′UTR region in the recombinant SARS-CoV-2 construct is about 2000 bp to about 3500 bp, including about any of 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, or any number in between.

In some embodiments, the total length of the 5′UTR region, the optional intervening sequence, and the 3′UTR region in the recombinant SARS-CoV-2 construct is between about 1,000 and about 10,000 bp. In some embodiments, the total length of the 5′UTR region, the optional intervening sequence, and the 3′UTR region in the recombinant SARS-CoV-2 construct is between about 1,000 and about 5,000 bp. In some embodiments, the total length of the 5′UTR region, the optional intervening sequence, and the 3′UTR region in the recombinant SARS-CoV-2 construct is between about 2,000 and about 3,500 bp. In some embodiments, the total length of the 5′UTR region, the optional intervening sequence, and the 3′UTR region in the recombinant SARS-CoV-2 construct is about 2,100 bp. In some embodiments, the total length of the 5′UTR region, the optional intervening sequence, and the 3′UTR region in the recombinant SARS-CoV-2 construct is about 3,500 bp.

The present disclosure also provides SARS-CoV-2 mutants, for example, interfering, conditionally replicating, SARS-CoV-2 deletion mutants, and related constructs. For example, the present disclosure provides SARS-CoV-2 deletion mutants have one or more of the deletions relative to the wild type SARS-CoV-2 sequence.

The present disclosure therefore also provides SARS-CoV-2 mutants. Such SARS-CoV-2 deletion mutants can have one or more deletions, for example at any location in SEQ ID NO:1. Such deletions can truncate or eliminate the sequence of any of the encoded polypeptides. For example, such deletions can truncate or delete the sequences identified by SEQ ID NOs: 2-19 or 22 or corresponding coding sequence. For example, such deletions of SARS-CoV-2 nucleic acids can reduce or eliminate the expression of any of the polypeptides encoded by the SARS-CoV-2 nucleic acids. However, in some cases certain regions of the SARS-CoV-2 genome should be retained (e.g., portions of the 5′UTR and/or the 3′UTR) and not be deleted.

The present disclosure identifies specific regions of the SARS-CoV-2 genome that should be retained and specific regions of the SARS-CoV-2 genome that can be deleted in order to provide interfering, conditionally replicating, SARS-CoV-2 deletion mutants and related constructs. For example, in order to function as therapeutic interfering particles (TIPs), SARS-CoV-2 deletion mutants can retain cis-acting elements such as, for example, the 5′UTR and the 3′UTR. In addition to retaining cis-acting elements, the interfering SARS-CoV-2 particles can, in some cases, retain portions of some of the SARS-CoV-2 proteins, such as the N protein or the spike receptor binding Si subunit (e.g., SEQ ID NO: 6).

Interfering SARS-CoV-2 particles (i.e., particles comprising a recombinant SARS-CpV-2 construct) that exhibit interference with wild type SARS-CoV-2 may, for example, compete for structural proteins that mediate viral particle assembly, or produce proteins that inhibit assembly of viral particles. For example, interfering SARS-CoV-2 particles that exhibit interference can have a deletion in the membrane-fusing S2 subunit of the spike protein (e.g., SEQ ID NO: 7). In some cases, interfering SARS-CoV-2 particles that exhibit interference can have one or more deletions in the RNA-dependent RNA polymerase (e.g., SEQ ID NO: 3). In some cases, interfering SARS-CoV-2 particles that exhibit interference can have one or more deletions in the M protein (membrane glycoprotein) (e.g., SEQ ID NO:12). In some cases, interfering SARS-CoV-2 particles that exhibit interference can have one or more deletions in the ssRNA-binding protein (e.g., SEQ ID NO: 22).

The deletion sizes of the SARS-CoV-2 deletion mutants and interfering, conditionally replicating, SARS-CoV-2 construct can vary. For example, the SARS-CoV-2 deletion mutants and interfering, conditionally replicating, SARS-CoV-2 construct can have one or more deletions, where each deletion has at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 15 bp, at least 20 bp, at least 25 bp, at least 30 bp, at least 40 bp of deletion.

In some cases, the deletion size can range, for example, from about 10 bp to about 5000 bp; from about 800 bp to about 2500 bp; from about 900 bp to about 2400 bp; from about 1000 bp to about 2300 bp; from about 1100 bp to about 2200 bp; from about 1200 bp to about 2100 bp; from about 1300 bp to about 2000 bp; from about 1400 bp to about 1900 bp; from about 1500 bp to about 1800 bp; or from about 1600 bp to about 1700 bp.

In some embodiments, the recombinant SARS-CoV-2 construct comprises a nucleic acid sequence derived from a SARS-CoV-2 viral genome. In some embodiments, the SARS-CoV-2 is WIV4, i.e., hCoV-19/WIV04/2019 or BetaCoV/WIV04/2019, or a SARS-CoV-2 virus having substantially the same genomic sequence (e.g., fewer than any one of 200, 100, 50, 20, 10, 5, 4, 3, 2, or 1 mutations) and phenotypes as SARS-CoV-2 WIV4.

In some embodiments, the SARS-COV-2 is a SARS-CoV-2 variant. Exemplary SARS-CoV-2 variants and spike protein mutations associated with these variants are shown in Table 1 below. The SARS-COV-2 variants described herein are named by the World Health Organization (WHO) or according to the Phylogenetic Assignment of Named Global Outbreak (PANGO) Lineages software. It is understood that the same variants may be referred to using different naming systems and algorithms in the art. SARS-CoV-2 variant classifications and definitions, as well as a list of known SARS-CoV-2 variants can be found at world wide web.cdc.gov/coronavirus/2019-ncov/variants/variant-classifications.html. In some embodiments, the SARS-CoV-2 variant may be any sequence with at least about 80% sequence homology to any of the above sequences, which may emerge from time to time. While the present application provides SEQ ID NO:1 as an exemplary SARS-CoV-2 genome sequence, it is to be understood that the present application also contemplates recombinant SARS-CoV-2 constructs derived from other SARS-CoV-2 viruses (such as SARS-CoV-2 variants described herein). Variants of SEQ ID NO:1 (or portions thereof, such as the 5′UTR and 3′UTR sequences of SED ID NO: 1) described herein therefore encompass corresponding sequences (such as corresponding 5′UTR and 3′UTR sequences) in other SARS-CoV-2 viruses (such as SARS-CoV-2 variants described herein).

TABLE 1
SARS-CoV-2 variants.

| | | | Mutation(s) in the |
|---|---|---|---|
| WHO label | PANGO lineage | Type | spike (S) protein |
| Alpha | B.1.1.7 and | Variant | 69del, 70del, 144del, |
| | Q lineages | being | (E484K*), (S494P*), |
| | | monitored | N501Y, A570D, D614G, |
| | | (VBM) | P681H, T716I, S982A, |
| | | | D1118H (K1191N*) |
| Beta | B.1.351 and | VBM | D80A, D215G, 241del, |
| | descendent | | 242del, 243del, K417N, |
| | lineages | | E484K, N501Y, D614G, |
| | | | A701V |
| Gamma | P.1 and | VBM | L18F, T20N, P26S, |
| | descendent | | D138Y, R190S, K417T, |
| | lineages | | E484K, N501Y, D614G, |
| | | | H655Y, T1027I |
| Epsilon | B.1.427 | VBM | L452R, D614G, |
| | B.1.429 | | S13I, W152C |
| Eta | B.1.525 | VBM | A67V, 69del, 70del, |
| | | | 144del, E484K, D614G, |
| | | | Q677H, F888L |
| Iota | B.1.526 | VBM | (L5F*), T95I, D253G, |
| | | | (S477N*), (E484K*), |
| | | | D614G, (A701V*) |
| Kappa | B.1.617.1 | VBM | (T95I), G142D, E154K, |
| | | | L452R, E484Q, D614G, |
| | | | P681R, Q1071H |
| N/A | B.1.617.3 | VBM | T19R, G142D, L452R, |
| | | | E484Q, D614G, P681R, |
| | | | D950N |
| Zeta | P.2 | VBM | E484K, (F565L*), |
| | | | D614G, V1176F |
| Mu | B.1.621, | VBM | D80G, 144del, F157S, |
| | B.1.621.1 | | L452R, D614G, (T791I*), |
| | | | (T859N*), D950H |
| Delta | B.1.617.2 | Variant of | T19R, (V70F*), T95I, |
| | and AY | Concern | G142D, E156-, F157-, |
| | lineages | (VOC) | R158G, (A222V*), |
| | | | (W258L*), (K417N*), |
| | | | L452R, T478K, D614G, |
| | | | P681R, D950N |
| Omicron | B.1.1.529 | VOC | A67V, del69-70, T95I, |
| | and BA | | del142-144, Y145D, |
| | lineages | | del211, L212I, |
| | | | ins214EPE, G339D, |
| | | | S371L, S373P, S375F, |
| | | | K417N, N440K, G446S, |
| | | | S477N, T478K, E484A, |
| | | | Q493R, G496S, Q498R, |
| | | | N501Y, Y505H, T547K, |
| | | | D614G, H655Y, N679K, |
| | | | P681H, N764K, D796Y, |
| | | | N856K, Q954H, N969K, |
| | | | L981F |

In some embodiments, the SARS-CoV-2 variant is selected from the group consisting of an Alpha (i.e., B.1.1.7 and Q) variant, a Beta (i.e., B.1.351) variant, a Gamma (i.e., P.1, also known as B.1.1.28.1) variant, an Epsilon (i.e., B.1.427 or B.1.429) variant, an Eta (i.e., B.1.525) variant, an Iota (i.e., B.1.526) variant, a Kappa (i.e., B.1.617.1) variant, a B. 1.617.3 variant, a Zeta (i.e., P.2) variant, a Mu (i.e., B.1.621 or B.1.621.1) variant, a Delta (i.e., B.1.617.2 or AY) variant, and an Omicron (i.e., B.1.1.529 or BA) variant. In some embodiments, the SARS-CoV-2 variant is a Delta variant, such as a B.1.617.2 variant, or an AY variant. In some embodiments, the SARS-CoV-2 variant is an Omicron variant, such as a B.1.529 variant or a BA variant. In some embodiments, the SARS-CoV-2 variant is selected from the group consisting of B.1.1.7, B.1.351, P.1, and B.1.617.2. In some embodiments, the SARS-CoV-2 variant has one or more mutations (e.g., insertion, deletion, and/or substitution) in the spike protein. In some embodiments, the one or more mutations in the spike protein may affect viral fitness, such as transmissibility, virulence, and/or drug resistance (e.g., resistance to neutralizing antibodies and/or resistance to a vaccine). In some embodiments, the one or more mutations in the spike protein do not substantially alter viral fitness. In some embodiments, the SARS-CoV-2 variant does not have a mutation in the spike protein.

### B. 5′UTR Region and 3′UTR Region

The recombinant SARS-CoV-2 constructs described herein comprise 5′UTR and 3′UTR regions derived from SARS-CoV-2 5′UTR and 3′UTR respectively.

In some embodiments, the 5′UTR region comprises stem loop 5 of SARS-CoV-2.

In some embodiments, the 5′UTR region of the recombinant SARS-CoV-2 construct comprises between about 100 and about 500 bp in total length, such as between about 100 and about 200 bp, between about 150 and about 250 bp, between about 200 and about 300 bp, between about 250 and about 350 bp, between about 300 and about 400 bp, between about 350 and about 450 bp, or between about 400 and about 500 bp in total length. In some embodiments, the 5′UTR region comprises greater than about 100 bp in total length, such as greater than any of about 150, 200, 250, 300, 350, 400, 450, 500 bp, or more, in total length. In some embodiments, the 5′UTR region comprises less than about 500 bp in total length, such as less than any of about 450, 400, 350, 300, 250, 200, 150, 100 bp, or fewer, in total length. In some embodiments, the 5′UTR region comprises about 265 bp in total length.

In some embodiments, the 5′UTR region of the recombinant SARS-CoV-2 construct comprises a fragment of SEQ ID NO: 1 or a variant thereof. In some embodiments, the 5′UTR region comprises at least about 30% sequence homology (such as at least any of about 35%, 40%, 45%, 50%, 55%, 60%, 65%, 70%, 75%, 80%, 85%, 90%, 91%, 92%, 93%, 94%, 95%, 96%, 97%, 98%, or 99% sequence homology) to nucleotides 1-265 of SEQ ID NO: 1 or a variant thereof. In some embodiments, the 5′UTR region comprises nucleotides 1-265 of SEQ ID NO: 1 or a variant thereof. In some embodiments, the 5′UTR region comprises nucleotides 1-265 of SEQ ID NO: 1.

In some embodiments, the recombinant SARS-CoV-2 construct comprises a 5′UTR region that comprises more than one copy of a SARS-CoV-2 5′UTR sequence or a variant thereof. For example, in some embodiments, the recombinant SARS-CoV-2 construct comprises any of about two, three, four, five, six, seven, eight, nine, ten, or more, copies of a SARS-CoV-2 5′UTR sequence or a variant thereof. In some embodiments, each 5′UTR sequence of the 5′UTR region of the recombinant SARS-CoV-2 construct comprises at least about 100 nucleotides of a SARS-CoV-2 5′UTR sequence or a variant thereof, such as at least any of about 150, 200, 250, 300, or more, nucleotides of a SARS-CoV-2 5′UTR sequence or a variant thereof. In some embodiments, each 5′UTR sequence of the 5′UTR region of the recombinant SARS-CoV-2 construct comprises less than about 300 nucleotides of a SARS-CoV-2 5′UTR sequence or a variant thereof, such as less than any of about 250, 200, 150, 100, or fewer, nucleotides of a SARS-CoV-2 5′UTR sequence or a variant thereof. In some embodiments, each 5′UTR sequence of the 5′UTR region comprises the same sequence of a SARS-CoV-2 5′UTR sequence or a variant thereof. In some embodiments, each 5′UTR sequence of the 5′UTR region comprises a sequence of different length of a SARS-CoV-2 5′UTR sequence or a variant thereof.

In some embodiments, the 3′UTR region of the recombinant SARS-CoV-2 construct comprises between about 100 and about 500 bp in total length, such as between about 100 and about 200 bp, between about 150 and about 250 bp, between about 200 and about 300 bp, between about 250 and about 350 bp, between about 300 and about 400 bp, between about 350 and about 450 bp, or between about 400 and about 500 bp in total length. In some embodiments, the 3′UTR region comprises greater than about 100 bp in total length, such as greater than any of about 150, 200, 250, 300, 350, 400, 450, 500 bp, or more, in total length. In some embodiments, the 3′UTR region comprises less than about 500 bp in total length, such as less than any of about 450, 400, 350, 300, 250, 200, 150, 100 bp, or fewer, in total length. In some embodiments, the 3′UTR region comprises about 228 bp in total length. In some embodiments, the 3′UTR region comprises about 196 bp in total length.

In some embodiments, the 3′UTR region of the recombinant SARS-CoV-2 construct comprises a fragment of SEQ ID NO: 1 or a variant thereof. In some embodiments, the 3′UTR region comprises at least about 30% sequence homology (such as at least any of about 35%, 40%, 45%, 50%, 55%, 60%, 65%, 70%, 75%, 80%, 85%, 90%, 91%, 92%, 93%, 94%, 95%, 96%, 97%, 98%, or 99% sequence homology) to nucleotides 29675-29870 of SEQ ID NO: 1 or a variant thereof. In some embodiments, the 5′UTR region comprises nucleotides 29675-29870 of SEQ ID NO: 1 or a variant thereof. In some embodiments, the 5′UTR region comprises nucleotides 29675-29870 of SEQ ID NO: 1. In some embodiments, the 3′UTR region comprises at least about 30% sequence homology (such as at least any of about 35%, 40%, 45%, 50%, 55%, 60%, 65%, 70%, 75%, 80%, 85%, 90%, 91%, 92%, 93%, 94%, 95%, 96%, 97%, 98%, or 99% sequence homology) to nucleotides 29675-29903 of SEQ ID NO: 1 or a variant thereof. In some embodiments, the 3′UTR region comprises nucleotides 29675-29903 of SEQ ID NO: 1 or a variant thereof. In some embodiments, the 3′UTR region comprises nucleotides 29675-29903 of SEQ ID NO: 1.

In some embodiments, the recombinant SARS-CoV-2 construct comprises a 3′UTR region that comprises more than one copy of a SARS-CoV-2 3′UTR sequence or a variant thereof. For example, in some embodiments, the recombinant SARS-CoV-2 construct comprises any of about two, three, four, five, six, seven, eight, nine, ten, or more, copies of a SARS-CoV-2 3′UTR sequence or a variant thereof. In some embodiments, each 5′UTR sequence of the 3′UTR region of the recombinant SARS-CoV-2 construct comprises at least about 100 nucleotides of a SARS-CoV-2 3′UTR sequence or a variant thereof, such as at least any of about 150, 200, 250, 300, or more, nucleotides of a SARS-CoV-2 3′UTR sequence or a variant thereof. In some embodiments, each 3′UTR sequence of the 3′UTR region of the recombinant SARS-CoV-2 construct comprises less than about 300 nucleotides of a SARS-CoV-2 3′UTR sequence or a variant thereof, such as less than any of about 250, 200, 150, 100, or fewer, nucleotides of a SARS-CoV-2 3′UTR sequence or a variant thereof. In some embodiments, each 3′UTR sequence of the 3′UTR region comprises the same sequence of a SARS-CoV-2 3′UTR sequence or a variant thereof. In some embodiments, each 3′UTR sequence of the 3′UTR region comprises a sequence of different length of a SARS-CoV-2 3′UTR sequence or a variant thereof.

### C. Intervening Sequence

In some aspects, the recombinant SARS-CoV-2 constructs described herein comprise an intervening sequence. In some embodiments, the intervening sequence comprises a SARS-CoV-2 sequence, a heterologous sequence, or a combination thereof. In some embodiments, the recombinant SARS-CoV-2 construct does not comprise an intervening sequence.

The intervening sequence is placed between the 5′UTR region and the 3′UTR region in the recombinant SARS-CoV-2 construct.

The recombinant SARS-CoV-2 construct may comprise an intervening sequence that is about 1 bp to about 29,000 bp in total length. In some embodiments, the intervening sequence is between about 1 and about 29,000 bp, such as between any of about 1 and about 100 bp, about 50 and about 250 bp, about 200 and about 500 bp, about 250 and about 750 bp, about 500 and about 1,000 bp, about 1,000 and about 10,000 bp, about 1,000 and about 5,000 bp, about 2,000 and about 3,500 bp, about 5,000 and about 15,000 bp, about 10,000 and about 20,000 bp, about 15,000 and about 25,000 bp, about 20,000 and about 29,000 bp. In some embodiments, the intervening sequence comprises greater than about 1 bp in total length, such as greater than any of about 10, 50, 100, 150, 200, 250, 500, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, or more, in total length. In some embodiments, the intervening sequence comprises less than about 29,000 bp in total length, such as less than any of about 28,500, 28,000, 27,500, 27,000, 26,500, 26,000, 25,500, 25,000, 24,500, 24,000, 23,500, 23,000, 22,500, 22,000, 21,500, 21,000, 19,500, 19,000, 18,500, 18,000, 17,500, 17,000, 16,500, 16,000, 15,500, 15,000, 14,500, 14,000, 13,500, 13,000, 12,500, 12,000, 11,500, 11,000, 10,500, 10,000, 9,500, 9,000, 8,500, 8,000, 7,500, 7,00, 6,500, 6,000, 5,500, 5,000, 4,500, 4,000, 3,500, 3,000, 2,500, 2,000, 1,500, 1,000, 500, 250, 200, 150, 100, 50, 10 bp, or fewer in total length.

### (i) SARS-CoV-2 Sequence

In some aspects, a recombinant SARS-CoV-2 construct comprises an intervening sequence comprising SARS-CoV-2 sequence or a variant thereof. As used herein, the term “SARS-CoV-2 sequence” includes any sequence derived from the SARS-CoV-2 viral genome, or a variant thereof. In some embodiments, the SARS-CoV-2 viral genome is from a wild-type SARS-CoV-2 strain. In some embodiments, the SARS-CoV-2 viral genome is from a SARS-CoV-2 strain selected from B.1.1.7 (alpha variant), B.1.351 (beta variant), P.1 (gamma variant), or B.1.617.2 (delta variant). It should be understood that the recombinant SARS-CoV-2 constructs of the present disclosure may comprise an intervening sequence comprising any known SARS-CoV-2 sequence, or variant thereof, or any currently unknown, future SARS-CoV-2 sequence, or variant thereof, and that such recombinant SARS-CoV-2 constructs are within the scope of the present disclosure.

In some embodiments, the SARS-CoV-2 sequence in the intervening sequence does not encode a gene product. In some embodiments, the SARS-CoV-2 sequence does not encode functional viral protein. In some embodiments, the SARS-CoV-2 sequence does not encode a functional viral RNA. A recombinant SARS-CoV-2s construct can include SARS-CoV-2 cis-acting sequence elements; and can include an alteration in the SARS-CoV-2 sequence such that alteration renders one or more encoded SARS-CoV-2 trans-acting polypeptides non-functional. By “non-functional” it is meant that the SARS-CoV-2 trans-activating polypeptide does not carry out its normal function, for example, due to truncation of or internal deletion within the encoded polypeptide, or due to lack of the polypeptide altogether. “Alteration” of a SARS-CoV-2 sequence includes deletion of one or more nucleotides and/or substitution of one or more nucleotides.

In some embodiments, the SARS-CoV-2 sequence comprises a portion of an ORF from the SARS-CoV-2 viral genome. In some embodiments, the SARS-CoV-2 sequence comprises a complete ORF from the SARS-CoV-2 viral genome, and one or more stop codons interspersed with the ORF resulting in no translation of the ORF or a non-functional viral protein. In some embodiments, the SARS-CoV-2 sequence comprises a mutation in the ORF that results in no translation of the ORF. In some embodiments, the SARS-CoV-2 sequence comprises a mutation in the ORF that results in a non-functional translated viral protein. For example, in some embodiments, the SARS-CoV-2 sequence comprises a frameshift mutation, a deletion, an insertion, a non-sense, or a missense mutation that results in no translation of the ORF or a non-functional viral protein.

In some embodiments, the recombinant SARS-CoV-2 construct does not include any intervening sequences not derived from the SARS-CoV-2 viral genome or a variant thereof. In some embodiments, the SARS-CoV-2 sequence is derived from any one of SEQ ID NOs: 1-22 or the corresponding coding sequence. In some embodiments, the SARS-CoV-2 sequence comprises the sequence of polyprotein ORF1ab (SEQ ID NO: 2), or a portion thereof. In some embodiments, the portion of polyprotein ORF1ab (SEQ ID NO: 2) does not encode a functional viral protein.

In some aspects, a recombinant SARS-CoV-2 construct comprises nucleotides 1-450 of SEQ ID NO: 1, or a variant thereof. In some embodiments, the recombinant SARS-CoV-2 construct comprises nucleotides 29543-29903 of SEQ ID NO: 1, or a variant thereof. In some embodiments, the recombinant SARS-CoV-2 construct comprises nucleotides 29543-29870 of SEQ ID NO: 1, or a variant thereof. In some embodiments, the recombinant SARS-CoV-2 construct comprises nucleotides 1-450 and nucleotides 29543-29903 of SEQ ID NO: 1, or a variant thereof. In some embodiments, the recombinant SARS-CoV-2 construct comprises nucleotides 1-450 and nucleotides 29543-29870 of SEQ ID NO: 1, or a variant thereof. In some embodiments, the recombinant SARS-CoV-2 construct comprises a 5′UTR region comprising the sequence of SEQ ID NO: 28, or a sequence comprising at least about 90% sequence identity (such as at least any of about 91%, 92%, 93%, 94%, 95%, 96%, 97%, 98%, or 99% sequence identity) to the sequence of SEQ ID NO: 28. In some embodiments, the recombinant SARS-CoV-2 construct comprises a 3′UTR region comprising the sequence of SEQ ID NO: 29, or a sequence comprising at least about 90% sequence identity (such as at least any of about 91%, 92%, 93%, 94%, 95%, 96%, 97%, 98%, or 99% sequence identity) to the sequence of SEQ ID NO: 29. In some embodiments, the recombinant SARS-CoV-2 construct comprises a 5′UTR region comprising the sequence of SEQ ID NO: 28, or a sequence comprising at least about 90% sequence identity (such as at least any of about 91%, 92%, 93%, 94%, 95%, 96%, 97%, 98%, or 99% sequence identity) to the sequence of SEQ ID NO: 28, and a 3′UTR region comprising the sequence of SEQ ID NO: 29, or a sequence comprising at least about 90% sequence identity (such as at least any of about 91%, 92%, 93%, 94%, 95%, 96%, 97%, 98%, or 99% sequence identity) to the sequence of SEQ ID NO: 29. In some embodiments, the total length of the 5′UTR region, the optional intervening sequence, and the 3′UTR region in the recombinant SARS-CoV-2 construct is about 2000 bp to about 3500 bp, such as about 2100 bp.

In some aspects, a recombinant SARS-CoV-2 construct comprises nucleotides 1-450 of SEQ ID NO: 1, or a variant thereof, wherein nucleotide 241 is mutated from a cytosine (C) to a thymine (T) (e.g., a C-241-T mutation within the 5′UTR). In some embodiments, the recombinant SARS-CoV-2 construct comprises nucleotides 29543-29903 of SEQ ID NO: 1, or a variant thereof. In some embodiments, the recombinant SARS-CoV-2 construct comprises nucleotides 29543-29870 of SEQ ID NO: 1, or a variant thereof. In some embodiments, the recombinant SARS-CoV-2 construct comprises nucleotides 1-450 and nucleotides 29543-29903 of SEQ ID NO: 1, or a variant thereof, wherein nucleotide 241 is mutated from a C to a T. In some embodiments, the recombinant SARS-CoV-2 construct comprises nucleotides 1-450 and nucleotides 29543-29870 of SEQ ID NO: 1, or a variant thereof, wherein nucleotide 241 is mutated from a C to a T. In some embodiments, the recombinant SARS-CoV-2 construct comprises a 5′UTR region comprising the sequence of SEQ ID NO: 32, or a sequence comprising at least about 90% sequence identity (such as at least any of about 91%, 92%, 93%, 94%, 95%, 96%, 97%, 98%, or 99% sequence identity) to the sequence of SEQ ID NO: 32. In some embodiments, the recombinant SARS-CoV-2 construct comprises a 3′UTR region comprising the sequence of SEQ ID NO: 29, or a sequence comprising at least about 90% sequence identity (such as at least any of about 91%, 92%, 93%, 94%, 95%, 96%, 97%, 98%, or 99% sequence identity) to the sequence of SEQ ID NO: 29. In some embodiments, the recombinant SARS-CoV-2 construct comprises a 5′UTR region comprising the sequence of SEQ ID NO: 32, or a sequence comprising at least about 90% sequence identity (such as at least any of about 91%, 92%, 93%, 94%, 95%, 96%, 97%, 98%, or 99% sequence identity) to the sequence of SEQ ID NO: 32, and a 3′UTR region comprising the sequence of SEQ ID NO: 29, or a sequence comprising at least about 90% sequence identity (such as at least any of about 91%, 92%, 93%, 94%, 95%, 96%, 97%, 98%, or 99% sequence identity) to the sequence of SEQ ID NO: 29. In some embodiments, the total length of the 5′UTR region, the optional intervening sequence, and the 3′UTR region in the recombinant SARS-CoV-2 construct is about 2000 bp to about 3500 bp, such as about 2100 bp.

In some aspects, a recombinant SARS-CoV-2 construct comprises nucleotides 1-1540 of SEQ ID NO: 1, or a variant thereof. In some embodiments, the recombinant SARS-CoV-2 construct comprises nucleotides 29191-29903 of SEQ ID NO: 1, or a variant thereof. In some embodiments, the recombinant SARS-CoV-2 construct comprises nucleotides 29191-29870 of SEQ ID NO: 1, or a variant thereof. In some embodiments, the recombinant SAR-CoV-2 construct comprises nucleotides 1-1540 and nucleotides 29191-29903 of SEQ ID NO: 1, or a variant thereof. In some embodiments, the recombinant SAR-CoV-2 construct comprises nucleotides 1-1540 and nucleotides 29191-29870 of SEQ ID NO: 1, or a variant thereof. In some embodiments, the recombinant SARS-CoV-2 construct comprises a 5′UTR region comprising the sequence of SEQ ID NO: 30, or a sequence comprising at least about 90% sequence identity (such as at least any of about 91%, 92%, 93%, 94%, 95%, 96%, 97%, 98%, or 99% sequence identity) to the sequence of SEQ ID NO: 30. In some embodiments, the recombinant SARS-CoV-2 construct comprises a 3′UTR region comprising the sequence of SEQ ID NO: 31, or a sequence comprising at least about 90% sequence identity (such as at least any of about 91%, 92%, 93%, 94%, 95%, 96%, 97%, 98%, or 99% sequence identity) to the sequence of SEQ ID NO: 31. In some embodiments, the recombinant SARS-CoV-2 construct comprises a 5′UTR region comprising the sequence of SEQ ID NO: 30, or a sequence comprising at least about 90% sequence identity (such as at least any of about 91%, 92%, 93%, 94%, 95%, 96%, 97%, 98%, or 99% sequence identity) to the sequence of SEQ ID NO: 30, and a 3′UTR region comprising the sequence of SEQ ID NO: 31, or a sequence comprising at least about 90% sequence identity (such as at least any of about 91%, 92%, 93%, 94%, 95%, 96%, 97%, 98%, or 99% sequence identity) to the sequence of SEQ ID NO: 31. In some embodiments, the total length of the 5′UTR region, the optional intervening sequence, and the 3′UTR region in the recombinant SARS-CoV-2 construct is about 2000 bp to about 3500 bp, such as about 3500 bp.

In some aspects, a recombinant SARS-CoV-2 construct comprises nucleotides 1-1540 of SEQ ID NO: 1, or a variant thereof, wherein nucleotide 241 is mutated from a C to a T (e.g., a C-241-T mutation within the 5′UTR). In some embodiments, the recombinant SAR-CoV-2 construct comprises nucleotides 29191-29903 of SEQ ID NO: 1 or a variant thereof. In some embodiments, the recombinant SARS-CoV-2 construct comprises nucleotides 29191-29870 of SEQ ID NO: 1, or a variant thereof. In some embodiments, the recombinant SAR-CoV-2 construct comprises nucleotides 1-1540 and nucleotides 29191-29903 of SEQ ID NO: 1, or a variant thereof, wherein nucleotide 241 is mutated from a C to a T. In some embodiments, the recombinant SAR-CoV-2 construct comprises nucleotides 1-1540 and nucleotides 29191-29870 of SEQ ID NO: 1, or a variant thereof, wherein nucleotide 241 is mutated from a C to a T. In some embodiments, the recombinant SARS-CoV-2 construct comprises a 5′UTR region comprising the sequence of SEQ ID NO: 33, or a sequence comprising at least about 90% sequence identity (such as at least any of about 91%, 92%, 93%, 94%, 95%, 96%, 97%, 98%, or 99% sequence identity) to the sequence of SEQ ID NO: 33. In some embodiments, the recombinant SARS-CoV-2 construct comprises a 3′UTR region comprising the sequence of SEQ ID NO: 31, or a sequence comprising at least about 90% sequence identity (such as at least any of about 91%, 92%, 93%, 94%, 95%, 96%, 97%, 98%, or 99% sequence identity) to the sequence of SEQ ID NO: 31. In some embodiments, the recombinant SARS-CoV-2 construct comprises a 5′UTR region comprising the sequence of SEQ ID NO: 33, or a sequence comprising at least about 90% sequence identity (such as at least any of about 91%, 92%, 93%, 94%, 95%, 96%, 97%, 98%, or 99% sequence identity) to the sequence of SEQ ID NO: 33, and a 3′UTR region comprising the sequence of SEQ ID NO: 31, or a sequence comprising at least about 90% sequence identity (such as at least any of about 91%, 92%, 93%, 94%, 95%, 96%, 97%, 98%, or 99% sequence identity) to the sequence of SEQ ID NO: 31. In some embodiments, the total length of the 5′UTR region, the optional intervening sequence, and the 3′UTR region in the recombinant SARS-CoV-2 construct is about 2000 bp to about 3500 bp, such as about 2100 bp.

### (ii) Heterologous Sequence

In some aspects, a recombinant SARS-CoV-2 construct comprises an intervening sequence comprising a heterologous sequence. In some embodiments, the heterologous sequence is a heterologous nucleotide sequence. “Heterologous” refers to a sequence that is not normally present in a wild-type SARS-CoV-2 genome, or a variant thereof, in nature. For example, in some cases, a recombinant SARS-CoV-2 construct does not include a heterologous sequence that encodes a gene product. In some embodiments, the heterologous sequence does not encode a functional protein. In some embodiments, the heterologous sequence does not encode a functional RNA.

In some embodiments, the recombinant SARS-CoV-2 construct comprises a heterologous sequence not derived from a SARS-CoV-2 sequence. In some embodiments, the heterologous sequence encodes one or more functional proteins or sequences. For example, a recombinant SARS-CoV-2 construct can include one or more marker sequences (such as barcode sequences or a unique molecular identifier sequence (UMI)), one or more nucleic acids encoding a detectable marker, a reporter protein, one or more promoters, one or more RNA transcription or translation initiation sites, one or more termination signals, or a combination thereof. The constructs can also include an origin of replication.

In some embodiments, the recombinant SARS-CoV-2 construct comprises a marker sequence that allows one to determine the presence or absence (or identity) of the recombinant SARS-Cov-2 construct, e.g., by PCR or nucleic acid sequencing. In some embodiments, the recombinant SARS-CoV-2 construct comprises a barcode sequence, such as a UMI sequence. As used herein, terms “barcode” and “UMI” are used interchangeably, and refer to a stretch of nucleotides having a sequence that uniquely tags the recombinant SARS-CoV-2 construct for future identification. For example, in some cases, a barcode cassette (from a pool of random barcode cassettes) can be added to the recombinant SARS-CoV-2 construct and the recombinant SARS-CoV-2 construct sequenced so that it is known which barcode sequence is associated with which particular construct. In this way, recombinant SARS-CoV-2 constructs can be tracked and accounted for by virtue of presence of the barcode. Identifying the presence of a short stretch of nucleotides using any convenient assay can easily be accomplished. Use of such barcodes is easier than isolating and sequencing recombinant SARS-CoV-2 constructs, e.g., using high throughput sequencing, a microarray, PCR, qPCR, or any other method that can detect the presence/absence of a barcode sequence.

In some cases, a barcode is added to the recombinant SARS-CoV-2 construct as a cassette. A barcode cassette is a stretch of nucleotides that have at least one constant region (a region shared by all members receiving the cassette) and a barcode region (i.e., a barcode sequence—a region unique to the members that receive the barcode such that the barcode uniquely marks the members of the library). For example, a barcode cassette can include (i) a constant region that is a primer site, which site is in common among the barcode cassettes used, and (ii) a barcode sequence that is a unique tag, e.g., can be a stretch of random sequence. In some cases, a barcode cassette includes a barcode region flanked by two constant regions (e.g., two different primer sites). As an illustrative example, in some cases a barcode cassette is a 60 bp cassette that includes a 20 bp random barcode flanked by 20 bp primer binding sites (e.g., see FIG. 4).

A barcode sequence can have any convenient length and is preferably long enough so that it uniquely marks the recombinant SARS-CoV-2 construct. In some cases, the barcode sequence has a length of from 15 bp to 40 bp (e.g., from 15-35 bp, 15-30 bp, 15-25 bp, 17-40 bp, 17-35 bp, 17-30 bp, or 17-25 bp). In some cases, the barcode sequence has a length of 20 bp. Likewise, a barcode cassette can have any convenient length, and this length depends on the length of the barcode sequence plus the length of the constant region(s). In some cases, the barcode cassette has a length of from 40 bp to 100 bp (e.g., from 40-80 bp, 45-100 bp, 45-80 bp, 45-70 bp, 50-100 bp, 50-80 bp, or 50-70 bp). In some cases, the barcode cassette has a length of 60 bp.

### D. Additional Construct Features

In some aspects, a recombinant SARS-CoV-2 construct provided herein comprises additional features that may be useful for interfering with SARS-CoV-2 replication. For examples, the recombinant SARS-CoV-2 construct may comprise sequences that confer increased construct stability, viral packing ability, etc.

In some embodiments, the recombinant SARS-CoV-2 construct comprises a packaging signal for SAR-CoV-2 or a variant thereof. During viral assembly, the viral RNA segments are incorporated into virons in a selective manner. Each viral RNA segment comprises a specific structure that mediates the packaging of the RNa into virons. The packaging signals play important roles in determining the virus replication, genome incorporation, and genetic reassortment of SARS-CoV-2 viruses. In some embodiments, the packaging signal comprises stem loop 5 in the SARS-CoV-2 5′UTR. Stem loop 5 in the SARS-CoV-2 5′UTR encodes a predicted packaging signal (Chen and Olsthoorn, 2010; Rangan et al., 2020) for packaging of SARS-CoV-2 viral RNA.

A recombinant SARS-CoV-2 construct described herein (for example a recombinant SARS-CoV-2 RNA construct) may comprise a modification that protects the construct. For example, in some embodiments, the recombinant SARS-CoV-2 construct comprises a 3′ modification (e.g., a modification added to the 3′ end of the nucleotide sequence of the recombinant SARS-CoV-2 construct) or a 5′ modification (e.g., a modification added to the 5′ end of the nucleotide sequence of the recombinant SARS-CoV-2 construct). In some embodiments, the recombinant SARS-CoV-2 construct comprises both a 3′ modification and a 5′ modification. A 3′ and/or a 5′ modification as described herein may facilitate the processing of an mRNA recombinant SARS-CoV-2 construct or a DNA recombinant SARS-CoV-2 construct.

In some embodiments, the recombinant SARS-CoV-2construct (such as a recombinant SARS-CoV-2 RNA construct) comprises a modification at any position in the construct, such as in the middle of the construct. Such modifications may block a 5′ or 3′ hydroxyl (—OH) group from reacting, confer resistance to 3′ exonuclease activity (e.g., nuclease resistance), stabilize the construct, and/or allow for further covalent modifications using amine or thiol groups. In some embodiments, the modifications are added to the recombinant SARS-CoV-2 construct after transcription of the recombinant SARS-CoV-2 construct (e.g., a post-transcriptional modification). In some embodiments, modifications are added to the recombinant SARS-CoV-2 construct before transcription of the recombinant SARS-CoV-2 construct. In some embodiments, the modifications are added to the recombinant SARS-CoV-2 construct before translation of the recombinant SARS-CoV-2 construct.

In some embodiments, the recombinant SARS-CoV-2 construct (such as a recombinant SARS-CoV-2 RNA construct) comprises a 3′ modification. In some embodiments, the recombinant SARS-CoV-2 construct comprises a 3′ extended sequence. In some embodiments, the 3′ extended sequence protects the 3′ end of the recombinant SARS-CoV-2 construct. In some embodiments, the 3′ extended sequence is an extended polyA sequence. In some embodiments, the recombinant SARS-CoV-2 construct comprises a signaling sequence for the addition of an extended polyA sequence. In some embodiments, 3′ extended sequence comprises a signaling sequence for the addition of an extended polyA sequence. In some embodiments, the extended polyA sequence comprises at least about 100 adenine nucleotides, such as at least any of about 150, 200, 250, 300, 350, 400, or more adenine nucleotides. The extended polyA sequence may stabilize the recombinant SARS-CoV-2 construct and/or allow the construct to be exported from the nucleus and translated into a protein by ribosomes in the cytoplasm.

In some embodiments, the recombinant SARS-CoV-2 construct (such as a recombinant SARS-CoV-2 RNA construct) comprises a 5′ modification. In some embodiments, the 5′ modification is a 5′ cap. The 5′ cap may allow for the creation of stable mRNA recombinant SARS-CoV-2 constructs, and allow for the translation of such constructs. In some embodiments, the 5′ cap regulates nuclear export of the recombinant SARS-CoV-2 construct, prevents degradation of the recombinant SARS-CoV-2 construct by exonucleases, promotes translation of the recombinant SARS-CoV-2 construct, and/or promotes 5′ proximal intron excision. In some embodiments, the 5′ cap is a 5′ methyl cap. In some embodiments, the 5′ methyl cap is a 7-methylguanylate cap.

In some embodiments, the recombinant SARS-CoV-2 construct comprises a modification that is not at the 3′ or 5′ end of the construct. In some embodiments, any of the nucleotides in the recombinant SARS-CoV-2 construct may modified. In some embodiments, the nucleotides may be synthetic and/or modified nucleic acid molecules, (e.g., including modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. Nucleotides suitable for use in the recombinant SARS-CoV-2 constructs of the present invention include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages.

In some embodiments, the recombinant SARS-CoV-2 construct is a vector. In some embodiments, the vector is a DNA vector. In some embodiments, the vector comprises a promoter. In some embodiments, the promoter is upstream of the 5′UTR region. In some embodiments, the promoter is a T7 promoter. In some embodiments, the vector comprises a 3′ extended polyA sequence. In some embodiments, the vector comprises a 3′ signal for polyA addition.

### E. Construct Properties

The present disclosure provides an interfering, recombinant SARS-CoV-2 construct. The interfering, recombinant SARS-CoV-2 constructs may be referred to as “TIPs”, such as when the recombinant SARS-CoV-2 construct is comprised in suitable vehicles for delivery, such as lipid nanoparticles or viral-like particles. Unlike SARS-CoV-2, recombinant SARS-CoV-2 construct is not replication competent by itself, but can replicate in the presence of SARS-CoV-2, which is a replication competent virus. For example, a subject recombinant SARS-CoV-2 construct, when present in a mammalian host, cannot, in the absence of SARS-CoV-2 (i.e., replication competent SARS-CoV-2), form infectious particles containing copies of itself. A subject recombinant SARS-CoV-2 construct can be packaged into an infectious particle inside a host cell when the appropriate polypeptides required for packaging are provided. The infectious particle can then infect other cells. In some embodiments, the recombinant SARS-CoV-2 construct can replicate more efficiently than SARS-CoV-2, thereby outcompeting the SARS-CoV-2. As a result, the SARS-CoV-2 viral load can be reduced in an individual infected with SARS-CoV-2.

A recombinant SARS-CoV-2 construct can be an RNA construct, an mRNA construct, or a DNA construct (e.g., a DNA copy of an RNA).

In some cases, a recombinant SARS-CoV-2 construct or a SARS-CoV-2 TIP, when present in a host cell (e.g., in a host cell in an individual) that is infected with SARS-CoV-2, replicates at a rate that is at least about 10%, at least about 20%, at least about 30%, at least about 40%, at least about 50%, at least about 75%, at least about 2-fold, at least about 2.5-fold, at least about 5-fold, at least about 10-fold, or greater than 10-fold (such as 20, 30, 40, or 50 fold), higher than the rate of replication of the wild-type SARS-CoV-2 in a host cell of the same type that does not comprise a subject recombinant SARS-CoV-2 construct or SARS-CoV-2 TIP.

In some cases, a recombinant SARS-CoV-2 construct or a SARS-CoV-2 TIP, when present in a host cell (e.g., in a host cell in an individual) that is infected with SARS-CoV-2, reduces the amount of SARS-CoV-2 transcripts in the cell by at least about 20%, at least about 30%, at least about 40%, at least about 50%, at least about 60%, at least about 70%, at least about 80%, or at least about 90%, compared to the amount of SARS-CoV-2 transcripts in a host cell that is infected with SARS-CoV-2, but does not comprise a subject recombinant SARS-CoV-2 construct or SARS-CoV-2 TIP.

In some embodiments, the recombinant SARS-CoV-2 construct genomic RNA is produced at a higher rate than SARS-CoV-2 genomic RNA (e.g., RNA from a replication competent SARS-CoV-2 that has infected the host cell) when present in a host cell infected with SARS-CoV-2, such that the ratio of the recombinant SARS-CoV-2 genomic RNA to the SARS-CoV-2 genomic RNA is greater than 1 in the cell. In some cases, a recombinant SARS-CoV-2 construct, when present in a host cell (e.g., in a host cell in an individual) that is infected with SARS-CoV-2, results in production of recombinant SARS-CoV-2 construct-encoded RNA such that the ratio (by weight, e.g., μg:pg) of recombinant SARS-CoV-2 construct-encoded RNA to SARS-CoV-2-encoded genomic RNA in the cytoplasm of the host cell is from at least about 1.5:1 to at least about 2:1 or greater than 2:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

In some cases, a recombinant SARS-CoV-2 construct, when present in a host cell (e.g., in a host cell in an individual) that is infected with SARS-CoV-2, results in production of recombinant SARS-CoV-2 construct-encoded RNA such that the ratio (e.g., molar ratio) of recombinant SARS-CoV-2 construct-encoded RNA to SARS-CoV-2-encoded genomic RNA in the cytoplasm of the host cell is greater than 1. In some cases, a recombinant SARS-CoV-2 construct, when present in a host cell (e.g., in a host cell in an individual) that is infected with a SARS-CoV-2, results in production of recombinant SARS-CoV-2 construct-encoded RNA such that the ratio (e.g., molar ratio) of recombinant SARS-CoV-2 construct-encoded RNA to SARS-CoV-2-encoded genomic RNA in the cytoplasm of the host cell is from at least about 1.5:1 to at least about 2:1 or greater than 2:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

A subject recombinant SARS-CoV-2 construct can exhibit a basic reproductive ratio (R₀) (also referred to as the “basic reproductive number”) that is greater than 1. R₀ is the number of daughter cells resulting from one infected parent cell (e.g., the number of cases one case generates on average over the course of its infectious period), usually characterized by an average of statistically significant number of repeated experiments. When R₀ is >1, the infection will be able to spread in a population (of cells or individuals). Thus, a subject recombinant SARS-CoV-2 construct has the capacity to spread from one cell to another or from one individual to another in a population. In some cases, the subject recombinant SARS-CoV-2 construct (or a subject recombinant SARS-CoV-2 particle) has an R₀ from about 2 to about 5, from about 5 to about 7, from about 7 to about 10, from about 10 to about 15, or greater than 15.

Any convenient method can be used to measure the ratio of recombinant SARS-CoV-2 construct-encoded RNA to SARS-CoV-2-encoded genomic RNA in the cytoplasm of the host cell. Suitable methods can include, for example, measuring transcript number directly via qRT-PCR (e.g., single-cell qRT-PCR) of both a recombinant SARS-CoV-2 construct-encoded RNA and a wild-type SARS-CoV-2-encoded RNA; measuring levels of a protein encoded by the interfering construct-encoded RNA and the SARS-CoV-2-encoded genomic RNA (e.g., via western blot, ELISA, mass spectrometry, etc.); and measuring levels of a detectable label associated with the recombinant SARS-CoV-2 construct-encoded RNA and the SARS-CoV-2-encoded genomic RNA (e.g., fluorescence of a fluorescent protein that is encoded by the RNA and is fused to a protein that is translated from the RNA). Such measurements can be performed, for example, after co-transfection, using any convenient cell type.

A recombinant SARS-CoV-2 construct as described herein, such as a recombinant SARS-CoV-2 construct comprised in a delivery vehicle (e.g., SARS-CoV-2 TIP), may have different transmission frequency as compared to SARS-CoV-2 (e.g., an infectious SARS-CoV-2, such as a replication competent SARS-CoV-2). SARS-CoV-2 is transmitted by exposure to infectious respiratory fluids. The principal mode by which people are infected with SARS-CoV-2 is through exposure to respiratory fluids carrying infectious virus. The term “transmission frequency” as used herein refers to the frequency of which a SARS-CoV-2 infection is passed from cell to cell in vitro, or the frequency of which a SARS-CoV-2 infection is passed from one person to another person or one animal to another animal in vivo. In some embodiments, the recombinant SARS-CoV-2 construct has the same transmission frequency as SARS-CoV-2. In some embodiments, the recombinant SARS-CoV-2 construct has lower (e.g., at least 1×. 2×. 3×. 4×, 5×, 6×, 7×, 8×, 9×, or 10× lower) transmission frequency than SARS-CoV-2.

In some embodiments, the recombinant SARS-CoV-2 construct has a higher transmission frequency than SARS-CoV-2.

In some embodiments, the recombinant SARS-CoV-2 construct-encoded RNA is packaged. In some embodiments, the recombinant SARS-CoV-2 construct-encoded RNA is unpackaged. In some cases, the recombinant SARS-CoV-2 construct-encoded RNA includes both packaged and unpackaged RNA.

In some embodiments, the recombinant SARS-CoV-2 construct is packaged with the same efficiency as SARS-CoV-2 when present in a host cell infected with SARS-CoV-2. In some embodiments, the recombinant SARS-CoV-2 construct is packaged with higher efficiency than SARS-CoV-2 when present in a host cell infected with SARS-CoV-2.

### III. Inhibitor of Transcription Regulating Sequences (TRSs)

In some aspects, provided herein is an inhibitor of SARS-CoV-2 transcription regulating sequences (TRSs). The inhibitor of SARS-CoV-2 TRSs may be an antisense oligonucleotide. In some embodiments, the inhibitor of SARS-CoV-2 TRSs is an antisense RNA. In some embodiments, the inhibitor of SARS-CoV-2 TRSs intervenes or interferes with SARS-CoV-2 infection. In some embodiments, the inhibitor of SARS-CoV-2 TRSs prevents progression of SARS-CoV-2 infection.

Transcription initiation is regulated in coronaviruses, such as SARS-CoV-2, by several types of consensus TRSs. These TRSs may comprise nucleic acid sequences that are capable of increasing or decreasing the expression of specific genes within the virus that are responsible for the initiation of transcription. The inhibition of such TRSs may therefore compromise the ability of SARS-CoV-2 to be transcribed, thereby intervening or interfering with SARS-CoV-2 infection.

In some embodiments, the TRS comprises the sequence of any one of SEQ ID NOs: 36-38, or variants thereof comprising at least about 90% sequence identity (such as at least any of about 91%, 92%, 93%, 94%, 95%, 96%, 97%, 98%, or 99% sequence identity) to the sequence of any one of SEQ ID NOs: 36-38. In some embodiments, the TRS comprises the sequence of TRS1-L: 5′-cuaaac-3′ (SEQ ID NO: 36), or a variant thereof comprising at least about 90% sequence identity (such as at least any of about 91%, 92%, 93%, 94%, 95%, 96%, 97%, 98%, or 99% sequence identity) to the sequence of SEQ ID NO: 36. In some embodiments, the TRS comprises the sequence of TRS2-L: 5′-acgaac-3′ (SEQ ID NO: 37), or a variant thereof comprising at least about 90% sequence identity (such as at least any of about 91%, 92%, 93%, 94%, 95%, 96%, 97%, 98%, or 99% sequence identity) to the sequence of SEQ ID NO: 37. In some embodiments, the TRS comprises the sequence of TRS3-L, 5′-cuaaacgaac-3′ (SEQ ID NO: 38), or a variant thereof comprising at least about 90% sequence identity (such as at least any of about 91%, 92%, 93%, 94%, 95%, 96%, 97%, 98%, or 99% sequence identity) to the sequence of SEQ ID NO: 38.

In some embodiments, the inhibitor of SARS-CoV-2 TRSs can bind to any one of SEQ ID NOs: 36-38, or a combination thereof. In some embodiments, the inhibitor of SARS-CoV-2 TRSs can bind to the sequence of SEQ ID NO: 36, or a variant thereof comprising at least about 90% sequence identity (such as at least any of about 91%, 92%, 93%, 94%, 95%, 96%, 97%, 98%, or 99% sequence identity) to the sequence of SEQ ID NO: 36. In some embodiments, the inhibitor of SARS-CoV-2 TRSs can bind to the sequence of SEQ ID NO: 37, or a variant thereof comprising at least about 90% sequence identity (such as at least any of about 91%, 92%, 93%, 94%, 95%, 96%, 97%, 98%, or 99% sequence identity) to the sequence of SEQ ID NO: 37. In some embodiments, the inhibitor of SARS-CoV-2 TRSs can bind to the sequence of SEQ ID NO: 38, or a variant thereof comprising at least about 90% sequence identity (such as at least any of about 91%, 92%, 93%, 94%, 95%, 96%, 97%, 98%, or 99% sequence identity) to the sequence of SEQ ID NO: 38. In some embodiments, the inhibitor of SARS-CoV-2 TRSs can bind to both the sequences of SEQ ID NO: 36 and 37. In some embodiments, the inhibitor of SARS-CoV-2 TRSs can bind to both the sequences of SEQ ID NO: 36 and 38. In some embodiments, the inhibitor of SARS-CoV-2 TRSs can bind to both the sequences of SEQ ID NO: 38 and 37. In some embodiments, the inhibitor of SARS-CoV-2 TRSs can bind to each of the sequences of SEQ ID NOs: 36-38.

In some embodiments, the inhibitor of SARS-CoV-2 TRSs comprises a sequence comprising ACGAACCUAAACACGAACCUAAAC (TRS1; SEQ ID NO: 25), or a variant thereof comprising at least about 90% sequence identity (such as at least any of about 91%, 92%, 93%, 94%, 95%, 96%, 97%, 98%, or 99% sequence identity) to SEQ ID NO: 25, ACGAACACGAACACGAACACGAAC (TRS2; SEQ ID NO: 26), or a variant thereof comprising at least about 90% sequence identity (such as at least any of about 91%, 92%, 93%, 94%, 95%, 96%, 97%, 98%, or 99% sequence identity) to SEQ ID NO: 26, CUAAACCUAAACCUAAACCUAAAC (TRS3; SEQ ID NO: 27), or a variant thereof comprising at least about 90% sequence identity (such as at least any of about 91%, 92%, 93%, 94%, 95%, 96%, 97%, 98%, or 99% sequence identity) to SEQ ID NO: 27, or a combination thereof. In some embodiments, the inhibitor of SARS-CoV-2 TRSs comprises a sequence consisting essentially of any of SEQ ID NOs: 25-27, or a variant thereof comprising at least about 90% sequence identity (such as at least any of about 91%, 92%, 93%, 94%, 95%, 96%, 97%, 98%, or 99% sequence identity) to any of SEQ ID NOs: 25-27, or a combination thereof. In some embodiments, the inhibitor of SARS-CoV-2 TRSs comprises each of SEQ ID NOs: 25-27. In some embodiments, the inhibitor of SARS-CoV-2 TRSs consists essentially of SEQ ID NOs: 25-27.

### IV. Packaged Viral-Like Particles, Vectors, and Cells

The present application also provides viral-like particles comprising the recombinant SARS-CoV-2 constructs described herein and a viral envelop protein. These viral-like particles are generated in the presence of SARS-CoV, and are packaged with the help of SARS-CoV-2, thereby resulting in SARS-CoV-2 TIPs.

The viral envelope protein may be a small, integral membrane protein that mediates several aspects of the virus, including assembly, budding, envelope formation, and pathogenies. In some embodiments, the viral envelope protein is a coronavirus envelope protein. In some embodiments, the viral envelope protein is a SARS-CoV-2 envelope protein.

Vectors comprising the recombinant SARS-CoV-2 constructs described herein are also provided. In some embodiments, the vectors comprise the nucleic acid sequence of the recombinant SARS-CoV-2 constructs described herein. Such vectors include, but are not limited to, DNA vectors, phage vectors, viral vectors, retroviral vectors, etc.

Cells (such as isolated cells) comprising the recombinant SARS-CoV-2 constructs and SARS-CoV-2 TIPs described herein are also contemplated. In some aspects, provided herein is an isolated cell comprising any of the recombinant SARS-CoV-2 constructs or SARS-CoV-2 TIPs provided herein.

In some embodiments, the recombinant SARS-CoV-2 constructs described herein may be comprised in prokaryotic cells, such as bacterial cells. In some embodiments, the recombinant SARS-CoV-2 constructs described herein may be comprised in eukaryotic cells, such as fungal cells (such as yeast cells), plant cells, insect cells, and mammalian cells. Exemplary eukaryotic cells include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The invention also provides host cells (such as isolated cells) comprising any of the recombinant SARS-CoV-2 constructs, SARS-CoV-2 TIPs, VLPs, or vectors described herein. In some embodiments, provided herein is a cell (such as an isolated cell) comprising a recombinant SARS-CoV-2 construct and/or SARS-CoV-2 TIP described herein. In some embodiments, provided herein is a cell (such as an isolated cell) comprising a nucleic acid sequence of any of the recombinant SARS-CoV-2 construct and/or SARS-CoV-2 TIP described herein. In some embodiments, the provided herein is a cell comprising a vector that contains the nucleic acid sequence of any of the recombinant SARS-CoV-2 construct and/or SARS-CoV-2 TIP described herein. Non-limiting examples of mammalian host cells include but not limited to COS, HeLa, and CHO cells. Suitable non-mammalian host cells include prokaryotes (such as E. coli or B. subtilis) and yeast (such as S. cerevisae, S. pombe; or K. lactis).

### V. Methods of Treatment

The present disclosure provides a method of reducing SARS-CoV-2 viral load in an individual. The method generally involves administering to the individual an effective amount of a recombinant SARS-CoV-2 construct, a recombinant SARS-CoV-2 construct comprised in a suitable delivery vehicle (e.g., a SARS-CoV-2 TIP), and/or a pharmaceutical formulation (also referred to herein as a “pharmaceutical composition”) comprising a recombinant SARS-CoV-2 construct or a recombinant SARS-CoV-2 construct comprised in a suitable delivery vehicle (e.g., a SARS-CoV-2 TIP). The terms “recombinant SARS-CoV-2 construct” and “TIP” may be used interchangeably herein, and refer to an interfering recombinant SARS-CoV-2 construct that is capable of interfering with SARS-CoV-2.

In some aspects, provided herein is a method of treating or preventing SARS-CoV-2 infection in an individual, comprising administering to the individual an effective amount of a pharmaceutical composition, such as any of the pharmaceutical compositions described herein. In some embodiments, the pharmaceutical composition is administered prior to (e.g., at least about 1, 2, 3, 4, 5, or 6 days prior to) the individual being infected with SARS-CoV-2. In some embodiments, the pharmaceutical composition is administered after (e.g., at least about 1, 2, 3, 4, 5, or 6 days after) the individual being infected with SARS-CoV-2. In some embodiments, the pharmaceutical composition is administered prior to (e.g., at least about 1, 2, 3, 4, 5, or 6 days prior to) the individual being tested positive with SARS-CoV-2 infection. In some embodiments, the pharmaceutical composition is administered after (e.g., at least about 1, 2, 3, 4, 5, or 6 days after) the individual being tested positive with SARS-CoV-2 infection. In some embodiments, the pharmaceutical composition is administered prior to (e.g., at least about 1, 2, 3, 4, 5, or 6 days prior to) the individual being in close contact with someone tested positive with SARS-CoV-2 infection. In some embodiments, the pharmaceutical composition is administered after (e.g., at least about 1, 2, 3, 4, 5, or 6 days after) the individual being in close contact with someone tested positive with SARS-CoV-2 infection. In some embodiments, the SARS-CoV-2 is from a SARS-CoV-2 strain selected from B.1.1.7, B.1.351, P.1, or B.1.617.2. In some embodiments, the pharmaceutical composition is administered as a single dose. In some embodiments, the pharmaceutical composition is administered as multiple doses. In some embodiments, the pharmaceutical composition is administered intranasally.

In some cases, a subject method involves administering to an individual in need thereof an effective amount of a recombinant SARS-CoV-2 construct or a SARS-CoV-2 interfering particle (e.g., SARS-CoV-2 TIP), or a pharmaceutical formulation comprising a subject recombinant SARS-CoV-2 construct or a subject SARS-CoV-2 interfering particle (e.g., SARS-CoV-2 TIP). In some cases, an effective amount of a subject interfering particle is an amount that, when administered to an individual in one or more doses, in monotherapy or in combination therapy, is effective to reduce SARS-CoV-2 virus load in the individual by at least about 10%, at least about 20%, at least about 25%, at least about 30%, at least about 40%, at least about 50%, at least about 60%, at least about 70%, at least about 80%, or greater than 80%, compared to the SARS-CoV-2 virus load in the individual in the absence of treatment with the interfering particle.

In some cases, a subject method involves administering to an individual in need thereof an effective amount of a recombinant SARS-CoV-2 construct and/or a SARS-CoV-2 interfering particle (e.g., SARS-CoV-2 TIP). In some embodiments, an “effective amount” of a subject interfering particle is an amount that, when administered to an individual in one or more doses, in monotherapy or in combination therapy, is effective to reduce symptoms of SARS-CoV-2 in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the individual in the absence of treatment with the interfering particle.

Any of a variety of methods can be used to determine whether a treatment method is effective. For example, determining whether the methods are effective can include evaluating whether the wild type SARS-CoV-2 viral load is reduced, determining whether the infected subject is producing antibodies against SARS-CoV-2, determining whether the infected subject is breathing without assistance, and/or determining whether the temperature of the infected subject is returning to normal. Measuring viral load can be by measuring the amount of SARS-CoV-2 in a biological sample, for example, using a polymerase chain reaction (PCR) with primers specific SARS-CoV-2 polynucleotide sequence; detecting and/or measuring a polypeptide encoded by SARS-CoV-2; using an immunological assay such as an enzyme-linked immunosorbent assay (ELISA) with an antibody specific for a SARS-CoV-2 polypeptide; or a combination thereof.

### A. Subjects to be Treated

The methods of the present disclosure are suitable for treating individuals who are suspected of having SARS-CoV-2 infection, and individuals who have SARS-CoV-2 infection, e.g., who have been diagnosed as having SARS-CoV-2 infection. The methods of the present disclosure are also suitable for use in individuals who have not been diagnosed as having SARS-CoV-2 infection (e.g., individuals who have been tested for SARS-CoV-2 and who have tested negative for SARS-CoV-2; and individuals who have not been tested), and who are considered at greater risk than the general population of contracting an SARS-CoV-2 infection (e.g., “at risk” individuals).

The methods of the present disclosure are suitable for treating individuals who are suspected of having SARS-CoV-2 infection, individuals who have SARS-CoV-2 infection (e.g., who have been diagnosed as having SARS-CoV-2 infection), and individuals who are considered at greater risk than the general population of contracting SARS-CoV-2 infection. Such individuals include, but are not limited to, individuals with healthy, intact immune systems, but who are at risk for becoming SARS-CoV-2 infected (“at-risk” individuals). In addition, such individuals include, but are not limited to, individuals that do not appear to have SARS-CoV-2 infection, but who may have reduced immune responses, heart disease, reduced lung capacity or a combination thereof (“at-risk” individuals). At-risk individuals include, but are not limited to, individuals who have a greater likelihood than the general population of becoming SARS-CoV-2 infection infected. Individuals at risk for becoming SARS-CoV-2 infected include, but are not limited to, essential services personnel such as medical personnel, emergency medical personnel, law enforcement, ambulance drivers, and public service drivers. Individuals at risk for becoming SARS-CoV-2 infected include, but are not limited to, older individuals (e.g., older than 65), immunocompromised individuals, individuals with heart disease, obese individuals, and individuals with other viral or bacterial infections. Individuals suitable for treatment therefore include individuals infected with, or at risk of becoming infected with SARS-CoV-2 or any variant thereof.

In some embodiments, the individual has a medical condition, a pre-existing condition, or a condition that reduces heart, lung, brain, or immune system function. In some embodiments, the individual is immunocompromised. In some embodiments, the individual is a human.

### VI. Formulations, Dosages, and Routes of Administration

Prior to introduction into a host, the recombinant SARS-CoV-2 construct or an interfering particle (e.g., SARS-CoV-2 TIP) can be formulated into various compositions for use in therapeutic and prophylactic treatment methods. In particular, the interfering construct or interfering particle can be made into a pharmaceutical composition by combination with appropriate pharmaceutically acceptable carriers or diluents and can be formulated to be appropriate for either human or veterinary applications. For simplicity, a subject interfering construct and a subject interfering particle are collectively referred to below as “active agent” or “active ingredient.”

In some aspects, provided herein is a pharmaceutical composition comprising any of the recombinant SARS-CoV-2 constructs described herein, and a pharmaceutically acceptable excipient. In some embodiments, the recombinant SARS-CoV-2 construct is present in a delivery vehicle (also referred to herein as “a pharmaceutically acceptable carrier”), thereby forming a SARS-CoV-2 TIP. As used herein, “a delivery vehicle” refers to a pharmaceutically acceptable substrate, composition, or vehicle used in the process of drug delivery, which may have one or more ingredients including, but not limited to, excipient(s), binder(s), diluent(s), solvent(s), filler(s), and/or stabilizer(s). A delivery vehicle according to the present disclosure may include, but is not limited to, a polymer-based delivery vehicle, a lipid nanoparticle, a nanoparticle, a liposome, a viral vector (such as any of the viral vectors described herein), a viral-like particle (VLP). In some embodiments, the delivery vehicle is a lipid nanoparticle.

A composition for use in a subject treatment method can comprise a SARS-CoV-2 interfering construct (e.g., a recombinant SARS-CoV-2 construct) or SARS-CoV-2 interfering particle (e.g., SARS-CoV-2 TIP) in combination with a pharmaceutically acceptable carrier. A variety of pharmaceutically acceptable carriers can be used that are suitable for administration. The choice of carrier will be determined, in part, by the particular vector, as well as by the particular method used to administer the composition. One skilled in the art will also appreciate that various routes of administering a composition are available, and, although more than one route can be used for administration, a particular route can provide a more immediate and more effective reaction than another route. Accordingly, there are a wide variety of suitable formulations of a subject interfering construct composition or a subject interfering particle composition.

A composition comprising a recombinant SARS-CoV-2 construct or subject interfering particle (e.g., SARS-CoV-2 TIP), alone or in combination with other antiviral compounds, can be made into a formulation suitable for parenteral administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be provided in unit dose or multidose sealed containers, such as ampules and vials, and can be stored in a freeze-dried (lyophilized) condition requiring only the addition of the sterile liquid carrier, for example, water, for injections, immediately prior to use. Injectable solutions and suspensions can be prepared from sterile powders, granules, and tablets, as described herein.

An aerosol formulation suitable for administration via inhalation also can be made. The aerosol formulation can be placed into a pressurized acceptable propellant, such as dichlorodifluoromethane, propane, nitrogen, and the like.

A formulation suitable for oral administration can be a liquid solution, such as an effective amount of a subject interfering construct or a subject interfering particle dissolved in diluents, such as water, saline, or fruit juice; capsules, sachets or tablets, each containing a predetermined amount of the active agent (a subject interfering construct or subject interfering particle), as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Similarly, a formulation suitable for oral administration can include lozenge forms, that can comprise the active ingredient in a flavor, usually sucrose and acacia or tragacanth; pastilles comprising the active ingredient (a subject interfering construct or subject interfering particle) in an inert base, such as gelatin and glycerin, or sucrose and acacia; and mouthwashes comprising the active agent in a suitable liquid carrier, as well as creams, emulsions, gels, and the like containing, in addition to the active agent, such carriers as are available in the art.

A formulation for rectal administration can be presented as a suppository with a suitable base comprising, for example, cocoa butter or a salicylate. A formulation suitable for vaginal administration can be presented as a pessary, tampon, cream, gel, paste, foam, or spray formula containing, in addition to the active ingredient, such carriers as are known in the art to be appropriate. Similarly, the active ingredient can be combined with a lubricant as a coating on a condom.

The dose administered to an animal, particularly a human, in the context of the present invention should be sufficient to effect a therapeutic response in the infected individual over a reasonable time frame. The dose will be determined by the potency of the particular interfering construct or interfering particle employed for treatment, the severity of the disease state, as well as the body weight and age of the infected individual. The size of the dose also will be determined by the existence of any adverse side effects that can accompany the use of the particular interfering construct or interfering particle employed. It is always desirable, whenever possible, to keep adverse side effects to a minimum.

The dosage can be in unit dosage form, such as a tablet, a capsule, a unit volume of a liquid formulation, etc. The term “unit dosage form” as used herein refers to physically discrete units suitable as unitary dosages for human and animal subjects, each unit containing a predetermined quantity of an interfering construct or an interfering particle, alone or in combination with other antiviral agents, calculated in an amount sufficient to produce the desired effect in association with a pharmaceutically acceptable diluent, carrier, or vehicle. The specifications for the unit dosage forms of the present disclosure depend on the particular construct or particle employed and the effect to be achieved, as well as the pharmacodynamics associated with each construct or particle in the host. The dose administered can be an “antiviral effective amount” or an amount necessary to achieve an “effective level” in the individual patient.

Generally, an amount of a subject interfering construct (e.g., recombinant SARS-CoV-2 construct) or a subject interfering particle (e.g., SARS-CoV-2 TIP) sufficient to achieve a tissue concentration of the administered construct or particle of from about 50 mg/kg to about 300 mg/kg of body weight per day can be administered, e.g., an amount of from about 100 mg/kg to about 200 mg/kg of body weight per day. In certain applications, e.g., topical, ocular or vaginal applications, multiple daily doses can be administered. Moreover, the number of doses will vary depending on the means of delivery and the particular interfering construct or interfering particle administered.

In some embodiments, a recombinant SARS-CoV-2 construct or interfering particle (e.g., SARS-CoV-2 TIP) (or composition comprising same) is administered in combination therapy with one or more additional therapeutic agents. Suitable additional therapeutic agents include agents that inhibit one or more functions of SARS-CoV-2 virus; agents that treat or ameliorate a symptom of SARS-CoV-2 virus infection; agents that treat an infection that may occur secondary to SARS-CoV-2 virus infection; and the like.

In some aspects, provided herein is a pharmaceutical composition comprising an inhibitor of SARS-CoV-2 transcription regulating sequences (TRSs), such as any of the inhibitors of SARS-CoV-2 TRSs described herein, and a pharmaceutically acceptable excipient.

In other aspects, provided herein is a pharmaceutical composition comprising a pharmaceutically acceptable excipient, (a) an inhibitor of SARS-CoV-2 TRSs that can bind to one of more of: SEQ ID NO: 36, SEQ ID NO: 37, SEQ ID NO: 38, or a combination thereof; and (b) a recombinant SARS-CoV-2 construct, the construct comprising: at least 100 nucleotides of a SARS-CoV-2 5′UTR, at least 100 nucleotides of a SARS-CoV-2 3′UTR, or a combination thereof. In some embodiments, provided herein is a pharmaceutical composition comprising a pharmaceutically acceptable excipient, (a) an inhibitor of SARS-CoV-2 TRSs comprising or consisting essentially of: SEQ ID NO: 25, SEQ ID NO: 26, SEQ ID NO: 27, or a combination thereof, or a combination thereof, and (b) a recombinant SARS-CoV-2 construct, the construct comprising: at least 100 nucleotides of a SARS-CoV-2 5′UTR, at least 100 nucleotides of a SARS-CoV-2 3′UTR, or a combination thereof.

### VII. Kits, Containers, Devices, Delivery Systems

Kits are described herein that include unit doses of the active agent (interfering recombinant SARS-CoV-2 construct, such as a recombinant SARS-CoV-2 construct and/or a SARS-CoV-2 TIP). The unit doses can be formulated for nasal, oral, transdermal, or injectable (e.g., for intramuscular, intravenous, or subcutaneous injection) administration. In such kits, in addition to the containers containing the unit doses will be an informational package insert describing the use and attendant benefits of the drugs in treating SARS-CoV-2 infection. Suitable active agents (a subject interfering construct or a subject interfering particle) and unit doses are those described herein above.

In some aspects, provided herein is a kit for treating or treating or preventing SARS-CoV-2 viral infection in an individual, comprising any of the pharmaceutical compositions described herein, and an instruction for carrying out any of the methods of treating or preventing SARS-CoV-2 infection in an individual described herein.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a subject active agent. For example, a subject delivery device comprises an injection device pre-loaded with a single dose of a subject active agent. A subject injection device can be re-usable or disposable.

Pen injectors are available. Exemplary devices which can be adapted for use in the present methods are any of a variety of pen injectors from Becton Dickinson, e.g., BD™ Pen, BD™ Pen II, BD™ Auto-Injector, a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

In some embodiments, a subject delivery system comprises a device for delivery to nasal passages or lungs. For example, the compositions described herein can be formulated for delivery by a nebulizer, an inhaler device, or the like.

Bioadhesive microparticles constitute still another drug delivery system suitable for use in the context of the present disclosure. This system is a multi-phase liquid or semi-solid preparation that preferably does not seep from the nasal passages. The substances can cling to the nasal wall and release the drug over a period of time. Many of these systems were designed for nasal use (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into liquids, creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance-free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### VIII. Methods of Generating Recombinant SARS-CoV-2 Constructs

The recombinant SARS-CoV-2 constructs (e.g., SARS-CoV-2 TIPs) described herein can be generated by molecular cloning methods known in the art. Non-limiting, exemplary methods are described herein.

a. Generating a Library of Cleaved (Linearized) SARS-CoV-2 DNAs

The methods described herein include generating a library of cleaved (linearized) SARS-CoV-2 DNAs from a population of circular SARS-CoV-2 DNAs. In some cases, the position of cleavage of the SARS-CoV-2 DNA population is random. For example, a transposon cassette can be inserted at random positions into a population of SARS-CoV-2 DNAs, where the transposon cassette includes a target sequence (recognition sequence) for a sequence specific DNA endonuclease. In such a case, the transposon cassette is being used as a vehicle for inserting a recognition sequence into the population of SARS-CoV-2 DNAs (at random positions). A sequence specific DNA endonuclease (one that recognizes the recognition sequence) can then be used to cleave the SARS-CoV-2 DNAs, thereby generating a library of cleaved (linearized) SARS-CoV-2 DNAs where members of the library are cut at different locations.

The term “transposon cassette” is used herein to mean a nucleic acid molecule that includes a “sequence of interest” flanked by sequences that can be used by a transposon to insert the sequence of interest into a SARS-CoV-2 DNA. Thus, in some cases, the “sequence of interest” is flanked by transposon compatible inverted terminal repeats (ITRs), i.e., ITRs that are recognized and utilized by a transposon. In cases where a transposon cassette is used as a vehicle for inserting one or more target sequences (for one or more sequence specific DNA endonucleases) into SARS-CoV-2 DNAs, the sequence of interest can include the one or more recognition sequences.

In some cases, the sequence of interest includes a selectable marker gene, for example, a nucleotide sequence encoding a selectable marker such as a gene encoding a protein that provides for drug resistance, for example, antibiotic resistance. In some cases, a sequence of interest includes a first copy and a second copy of a recognition sequence for a first sequence specific DNA endonuclease (e.g., a first meganuclease). In some cases, a sequence of interest includes a selectable marker gene flanked by a first and second recognition sequence for a sequence specific DNA endonuclease (e.g., meganuclease). In some such cases, the first recognition sequence and the second recognition sequence are identical and can be considered a first copy and a second copy of a recognition sequence. In some such cases, the first recognition sequence is different than the second recognition sequence. In some cases, the first recognition sequence and second recognition sequence (e.g., first and second copies of a recognition sequence) flank a selectable marker gene, for example, one that encodes a drug resistance protein such as an antibiotic resistance protein. In some embodiments, a subject transposon cassette includes a first copy and a second copy of a recognition sequence for a first meganuclease; and a first copy and a second copy of a recognition sequence for a second meganuclease.

As noted above, a subject transposon cassette includes a sequence of interest flanked by transposase compatible inverted terminal repeats (ITRs). The ITRs can be compatible with any desired transposase, for example, a bacterial transposase such as Tn3, Tn5, Tn7, Tn9, Tn10, Tn903, Tnl681, and the like; and eukaryotic transposases such as Tcl/mariner super family transposases, piggyBac superfamily transposases, hAT superfamily transposases, Sleeping Beauty, Frog Prince, Minos, Himari, and the like. In some cases, the transposase compatible ITRs are compatible with (i.e., can be recognized and utilized by) a Tn5 transposase. Some of the methods provided herein include a step of inserting a transposase cassette into a SARS-CoV-2 DNA. Such a step includes contacting the SARS-CoV-2 DNA and the transposon cassette with a transposase. In some cases, this contacting occurs inside of a cell such as a bacterial cell, and in some cases this contacting occurs in vitro outside of a cell. As the transposase compatible ITRs listed above are suitable for compositions and methods disclosed herein, so too are the transposases. As such, suitable transposases include but are not limited to bacterial transposases such as Tn3, Tn5, Tn7, Tn9, Tn10, Tn903, Tnl681, and the like; and eukaryotic transposases such as Tcl/mariner super family transposases, piggyBac superfamily transposases, hAT superfamily transposases, Sleeping Beauty, Frog Prince, Minos, Himarl, and the like. In some cases, the transposase is a Tn5 transposase.

In some embodiments, a subject method includes a step of inserting a target sequence (e.g., one or more target sequences) for a sequence specific DNA endonuclease (e.g., one or more sequence specific DNA endonucleases) into a population of circular SARS-CoV-2 DNAs, thereby generating a population of sequence-inserted circular SARS-CoV-2 DNAs. In some cases, the inserting step is carried out by inserting a transposon cassette that includes the target sequence (e.g., the one or more target sequences), thereby generating a population of transposon-inserted circular SARS-CoV-2 DNAs. In some cases, the transposon cassette includes a single recognition sequence (e.g., in the middle or near one end of the transposon cassette) and can therefore be used to introduce a single recognition sequence into the population of SARS-CoV-2 DNAs. In some cases, the transposon cassette includes more than one recognition sequences (e.g., a first and a second recognition sequence). In some such cases, the first and second recognition sequences are positioned at or near the ends of the transposon cassette (e.g., within 20 bases, 30 bases, 50 bases, 60 bases, 75 bases, or 100 bases of the end) such that cleavage of the first and second recognition sequences effectively removes the transposon cassette (or most of the transposon cassette) from the SARS-CoV-2 DNA, while simultaneously generating a linearized SARS-CoV-2 DNA, and therefore generating the desired library of cleaved (linearized) SARS-CoV-2 DNAs where members of the library are cut at different locations.

In some cases when the transposon cassette include first and second recognition sequences, the first and second recognition sequences are the same, and are therefore first and second copies of a given recognition sequence. In some such cases, the same sequence specific DNA endonuclease (e.g., restriction enzyme, meganuclease, programmable genome editing nuclease) can then be used to cleave at both sites. In some embodiments, the transposon cassette includes a first and a second recognition sequence where the first and second recognition sequences are not the same. In some such cases, a different sequence specific DNA endonuclease (e.g., restriction enzyme, meganuclease, programmable genome editing nuclease) is used to cleave the two sites (e.g., the library of transposon-inserted SARS-CoV-2 DNAs can be contacted with two sequence specific DNA endonucleases). However, in some cases one sequence specific DNA endonuclease can still be used. For example, in some cases two different guide RNAs can be used with the same CRISPR/Cas protein. As another example, in some cases a given sequence specific DNA endonuclease can recognize both recognition sequences.

In some cases, the population of circular SARS-CoV-2 DNAs (e.g., plasmids) are present inside of host cells (e.g., bacterial host cells such as E. coli) and the step of inserting a transposon cassette takes place inside of the host cell. For example, the methods can include introducing a transposase and/or a nucleic acid encoding a transposase into a selected cell or expression of a transposase within the cell from an existing expression cassette that encodes the transposase, and the like. In some such cases, a subject method can include a selection/growth step in the host cell. For example, if the transposon cassette includes a drug resistance marker, the host cells can be grown in the presence of drug to select for those cells harboring a transposon-inserted circular target DNA.

Once a population of transposon-inserted circular SARS-CoV-2 DNAs is generated (and in some cases after a selection/growth step in the host cells), the population can be isolated/purified from the host cells prior to the next step (e.g., prior to contacting them with a sequence specific DNA endonuclease).

Because the circular SARS-CoV-2 DNAs can be small circular DNAs (e.g., less than 50 kb), a selection and growth step in bacteria can in some cases be avoided through the use of in vitro rolling circle amplification (RCA). For example, after repair of nicked target DNA post-transposition, a highly-processive and strand-displacing polymerase (e.g., phi29 DNA polymerase), along with primers specific to the inserted transposon cassette, can be used to selectively amplify insertion mutants from the pool of circular plasmids. In other words, such a step can circumvent amplifying DNA through bacterial transformation. Use of RCA can decrease the time required for growth/selection of bacteria and can avoid biasing the library towards clones that do not impede bacterial growth. Non-random cleavage

As noted above, in some cases the position of cleavage of the SARS-CoV-2 DNA population is random, however in some cases the position of cleavage is not random. For example, a population of SARS-CoV-2 DNAs can be distributed (e.g., aliquoted) into different vessels (e.g., different tubes, different wells of a multi-well plate etc.). If a specific sequence of interest is selected within the SARS-CoV-2 genomic sequence, then that sequence of interest can be cleaved within the circular SARS-CoV-2 DNAs. Separate aliquots of circular SARS-CoV-2 DNAs can be placed within different vessels (e.g., wells of the multi-well plate) and the different aliquots of circular SARS-CoV-2 DNAs can be cleaved at different pre-determined locations by using a programmable sequence specific endonuclease. For example, if a CRISPR/Cas endonuclease (e.g., Cas9, Cpfl, and the like) is used, guide RNAs can readily be designed to target any desired sequence within the SARS-CoV-2 genome (e.g., while taking protospacer adj acent motif (PAM) sequence requirements into account in some cases). For example, guide RNAs can be tiled at any desired spacing along the circular SARS-CoV-2 DNAs (e.g., every 5 nucleotides (nt), every 10 nt, every 20 nt, every 50 nt—overlapping, non-overlapping, and the like). The circular SARS-CoV-2 DNAs in each vessel (e.g., each well) can be contacted with one of the guide RNAs in addition to the CRISPR/Cas endonuclease. In this way, a library of cleaved SARS-CoV-2 DNAs can be generated where members of the library are separated from one another because they are in separate vessels. As would be understood by one of ordinary skill in the art, in some cases, one would take PAM sequences into account when designing guide RNAs, and therefore the spacing between guide RNA target sites can be a function of PAM sequence constraints, and consistent spacing across a given target sequence would not necessarily be possible in some cases. However, different CRISPR/Cas endonucleases (e.g., even the same protein, such as Cas9, isolated from different species) can have different PAM requirements, and thus, the use of more than one CRISPR/Cas endonuclease can in some cases relieve at least some of the constraints imposed by PAM requirements on available target sites. Further steps of the method can then be carried out separately (e.g., in separate vessels, in separate wells of a multi-well plate), or at any step, members can be pooled and treated together in one vessel. As an illustrative but non-limiting example, one could use 96 different guide RNAs (or 384 different guide RNAs) to cleave aliquots of circular SARS-CoV-2 DNAs in 96 different wells of a 96-well plate (or 384 different wells of a 384 well plate), to generate 96 members (or 384 members) of a library where each member is cleaved at a different site. The cleavage sites can be designed by the user prior to starting the method. The exonuclease step (chew back) can then be performed in separate wells (e.g., by aliquoting exonuclease to each well), or two more wells can be pooled prior to adding exonuclease to the pool. Circular SARS-CoV-2 DNAs

A circular SARS-CoV-2 DNA of a population of circular SARS-CoV-2 DNAs can be any circular SARS-CoV-2 DNA and can be generated from any isolate of SARS-CoV-2. In some cases, the circular SARS-CoV-2 DNAs are plasmid DNAs.

For example, in some cases, the circular SARS-CoV-2 DNAs include an origin of replication (ORI). In some cases, the circular SARS-CoV-2 DNAs include a drug resistance marker (e.g., a nucleotide sequence encoding a protein that provides for drug resistance). In some embodiments, a population of circular SARS-CoV-2 DNAs are generated from a population of linear DNA molecules (e.g., via intramolecular ligation). For example, a subject method can include a step of circularizing a population of linear SARS-CoV-2 DNA molecules (e.g., a population of PCR products, a population of linear viral SARS-CoV-2 genomes, a population of products from a restriction digest, etc.) to generate a population of circular SARS-CoV-2 DNAs. In some cases, members of such a population are identical (e.g., many copies of a PCR product or restriction digest can be used to generate a population of SARS-CoV-2 DNAs, where each circular DNA is identical). In some cases, members of such a population of circular SARS-CoV-2 DNAs can be different from one another. For example, the population of circular SARS-CoV-2 DNAs can be generated from two or more different SARS-CoV-2 isolates or be generated from different SARS-CoV-2 PCR products or be generated from different restriction digest products of SARS-CoV-2.

In some cases, the population of circular SARS-CoV-2 DNAs can itself be a deletion library. For example, the population of circular SARS-CoV-2 DNAs can be a library of known deletion mutants (e.g., known viral deletion mutants). As another example, if two rounds of a subject method are performed, the starting population of SARS-CoV-2 DNAs for the second round can be a deletion library (e.g., generated during a first round of deletion) where members of the library include deletions of different sections of DNA relative to other members of the library. Such a library can serve as a population of circular SARS-CoV-2 DNAs, e.g., a transposon cassette can still be introduced into the population. Performing a second round of deletion in this manner can therefore generate constructs with deletions at multiple different entry points. As an illustrative example, for a SARS-CoV-2 DNA of about 29-30 kb (kilobases) in length, the first round of deletion might have deleted bases 2000 through 2650 for a one member (of the library that was generated), of which multiple copies would likely be present. A second round of deletion might generate two new members, both of which are generated from copies of the same deletion member. Thus, for example, one new member might be generated with bases 3500 through 3650 deleted (in addition to bases 2000 through 2650), while a second new member might be generated with bases 1500 through 1580 deleted (in addition to bases 2000 through 2650). Thus, multiple rounds of deletion (e.g., 2, 3, 4, 5, etc.) can produce complex deletion libraries. In some cases, more than one round of library generation is performed where the second round includes the insertion of a transposon cassette, e.g., as described above.

For example, in some cases, a first round of deletion is performed using a CRISPR/Cas endonuclease to generate the cleaved linear SARS-CoV-2 DNAs by targeting the CRISPR/Cas endonuclease to pre-selected sites within the population of circular SARS-CoV-2 DNAs (e.g., by designing guide RNAs, e.g., at pre-selected spacing, to target one or more SARS-CoV-2 sequences of interest). After exonuclease treatment and circularization to generate a first library of circularized deletion DNAs, the library of circularized deletion DNAs is used as input (as a population of circular SARS-CoV-2 DNAs) for a second round of deletion. Thus, one or more target sequences for one or more sequence specific DNA endonucleases (e.g., one or more meganucleases) is inserted (e.g., at random positions via a transposon cassette) into the library of circularized SARS-CoV-2 deletion DNAs to generate a population of transposon-inserted circular SARS-CoV-2 DNAs, and the method is continued. In some such cases, the first round of deletion might only target a small number of locations of interest for deletion (one location, e.g., using only one guide RNA that targets a particular location; or a small number of locations, e.g., using a small number of guide RNAs to target a small number of locations), while the second round is used to generate deletion constructs that include the first deletion plus a second deletion.

In some cases, the circular SARS-CoV-2 DNAs include the whole viral genome. In other cases, the circular SARS-CoV-2 DNAs include a partial SARS-CoV-2 viral genome. Thus, in some cases the subject methods are used to generate a library of viral deletion mutants. In some such cases, a library of generated viral deletion mutants can be considered a library of potential defective interfering particles (DIPs). DIPs are mutant versions of SARS-CoV-2 viruses that include genomic deletions such that they are unable to replicate except when complemented by wild-type virus replicating within the same cell. Defective interfering particles (DIPs) can arise naturally because viral genomes encode both cis-acting and trans-acting elements. Trans-acting elements (trans-elements) code for gene products, such as capsid proteins or transcription factors, and cis-acting elements (cis-elements) are regions of the viral genome that interact with trans-element products to achieve productive viral replication including viral genome amplification, encapsidation, and viral egress. In other words, the SARS-CoV-2 viral genome of a DIP can still be copied and packaged into viral particles if the missing (deleted) trans-elements are provided in trans (e.g., by a co-infecting virus). In some cases, a DIP can be used therapeutically to reduce viral infectivity of a co-infecting virus, e.g., by competing for and therefore diluting out the available trans-elements. In such cases, where a SARS-CoV-2 DIP can be used as a therapeutic (e.g., as a treatment for Covid-19 infections), that SARS-CoV-2 DIP can be referred to as a therapeutic interfering particle (TIP).

While DIPs may arise naturally, methods of this disclosure can be used to generate useful types of SARS-CoV-2 DIPs, for example, by generating a deletion library of viral SARS-CoV-2 genomes. DIPs can then be identified from such a deletion library by sequencing the library members to identify those predicted to be DIPs. Alternatively, or additionally, a generated deletion library can be screened. For example, a library of SARS-CoV-2 DIPs can be introduced into cells, to identify those members with viral genomes having the desired function. Additional description of DIPs and TIPs and uses thereof is provided in U.S. Patent Application Publication No. 20160015759, the disclosure of which is incorporated by reference herein in its entirety. Thus, in some cases a subject method includes introducing members of a library of generated SARS-CoV-2 deletion constructs into a target cell (e.g., a eukaryotic cell, such as a mammalian cell, such as a human cell) and assaying for infectivity. In some such cases, the assaying step also includes complementation of the library members with a co-infecting SARS-CoV-2 virus.

Such introducing is meant herein to encompass any form of introduction of nucleic acids into cells (e.g., electroporation, transfection, lipofection, nanoparticle delivery, viral delivery, and the like). For example, such ‘introduction’ encompasses infecting mammalian cells in culture (e.g., with members of a generated library of circularized SARS-CoV-2 deletion viral DNAs that can be encapsulated as viral particles that contain viral genomes encoded by the members of the generated library of circularized deletion viral DNAs).

In some cases, a method includes generating from a library of SARS-CoV-2 deletion DNAs, at least one of: linear double stranded DNA (dsDNA) products, linear single stranded DNA (ssDNA) products, linear single stranded RNA (ssRNA) products, and linear double stranded RNA (dsRNA) products. Thus in some such cases, a subject method includes introducing such linear dsDNA products, linear ssDNA products, linear ssRNA products, and/or linear dsRNA products into mammalian cells (e.g., via any convenient method for introducing nucleic acids into cells, including but not limited to electroporation, transfection, lipofection, nanoparticle delivery, viral delivery, and the like).

Such methods can also include assaying for viral infectivity. Assaying for viral infectivity can be performed using any convenient method. Assaying for viral infectivity can be performed on the cells into which the members of the library of circularized SARS-CoV-2 deletion DNAs (and/or at least one of: linear double stranded DNA (dsDNA) products, linear single stranded DNA (ssDNA) products, linear single stranded RNA (ssRNA) products, and linear double stranded RNA (dsRNA) products generated from the library of circularized deletion DNAs) are introduced. For example, in some cases the members and/or products are introduced as encapsulated particles. In some cases, members of the library of circularized

SARS-CoV-2 deletion DNAs (and/or at least one of: linear dsDNA products, linear ssDNA products, linear ssRNA products, and linear dsRNA products generated from the library of circularized SARS-CoV-2 deletion DNAs) are introduced into a first population of cells (e.g., mammalian cells) in order to generate viral particles, and the viral particles are then used to contact a second population of cells (e.g., mammalian cells). Thus, as used herein, unless otherwise explicitly described, the phrase “assaying for viral infectivity” encompasses both of the above scenarios (e.g., encompasses assaying for infectivity in the cells into which the members and/or products were introduced, and also encompasses assaying the second population of cells as described above).

In some embodiments a subject method (e.g., a method of generating and identifying a DIP) includes, after generating a deletion library (e.g., a library of circularized SARS-CoV-2 deletion DNAs), a high multiplicity of infection (MOI) screen (e.g., utilizing a MOI of >2). As used herein, a “high MOI” is a MOI of 2 or more (e.g., 2.5 or more, 3 or more, 5 or more, etc.). In some cases, a subject method uses a high MOI. Thus, in some cases, a subject method uses a MOI (a high MOI) of 2 or more, 3 or more, or 5 or more. In some cases, a subject method uses a MOI (a high MOI) in a range of from 2-150 (e.g., from 2-100, 2-80, 2-50, 2-30, 3-150, 3-100, 3-80, 3-50, 3-30, 5-150, 5-100, 5-80, 5-50, or 5-30). In some cases, a subject method uses a MOI (a high MOI) in a range of from 3-100 (e.g., 5-100). At high MOI, many (if not all) cells are infected by more than one virus, which allows for complementation of defective viruses by wild-type counterparts. Repeated passaging of deletion mutant libraries at high-MOI can select for mutants that can be mobilized effectively by a wild type SARS-CoV-2. For example, in some cases the method includes infecting mammalian cells in culture with members of the library of circularized SARS-CoV-2 deletion viral DNAs at a high multiplicity of infection (MOI), culturing the infected cells for a period of time ranging from 12 hours to 2 days (e.g., from 12 hours to 36 hours or 12 hours to 24 hours), adding naive cells to the to the culture, and harvesting virus from the cells in culture. However, this screening step can in some cases select for DIPs/TIPs which can be mobilized effectively by the wild-type virus but are cytopathic in the absence of the wild-type coinfection.

Thus, in some embodiments a subject method (e.g., a method of generating and identifying a DIP) includes a more stringent screen (referred to herein as a “low multiplicity of infection (MOI) screen”). As used herein, a “low MOI” includes use of a MOI of less than 1 (e.g., less than 0.8, less than 0.6, etc.). In some cases, a subject method uses a low MOI. Thus, in some cases, a subject method uses a MOI (a low MOI) of less than 1 (e.g., less than 0.8, less than 0.6). In some cases, a subject method uses a MOI (a low MOI) in a range of from 0.001-0.8 (e.g., from 0.001-0.6, 0.001-0.5, 0.005-0.8, 0.005-0.6, 0.01-0.8, or 0.01-0.5). In some cases, a subject method uses a MOI (a low MOI) in a range of from 0.01-0.5. For example, a low-MOI infection of target cells with a deletion library (e.g., utilizing a MOI of <1) can be alternated with a high-MOI infection of the transduced population with wild-type virus (e.g., SARS-CoV-2) to mobilize DIPs to naive cells.

In some cases, cells with one or more SARS-CoV-2 or one or more SARS-CoV-2 deletion DNAs can be propagated in the presence of a drug to test whether further rounds of replication occur. During the recovery period, cells infected with wild type virus (e.g., SARS-CoV-2 infected cells) will be killed, but cells transduced by well-behaving mutants (which do not produce cell-killing trans-factors) will be maintained. In this fashion, mutants can be selected that do not kill their transduced host-cell but that can mobilize during wild-type virus coinfection. Thus, in some cases a subject method includes infecting mammalian cells in culture with members of the library of circularized deletion SARS-CoV-2 viral DNAs at a low multiplicity of infection (MOI), culturing the infected cells in the presence of an inhibitor of viral replication for a period of time ranging from 1 day to 6 days (e.g., from 1 day to 5 days, from 1 day to 4 days, from 1 day to 3 days, or from 1 day to 2 days), infecting the cultured cells with functional SARS-CoV-2 virus at a high MOI, culturing the infected cells for a period of time ranging from 12 hours to 4 days (e.g., 12 hours to 72 hours, 12 hours to 48 hours, or 12 hours to 24 hours), and harvesting virus from the cultured cells.

In some embodiments, a subject method includes (a) inserting a target sequence for a sequence specific DNA endonuclease into a population of circular SARS-CoV-2 viral DNAs, to generate a population of sequence-inserted SARS-CoV-2 DNAs; (b) contacting the population of sequence-inserted SARS-CoV-2 DNAs with the sequence specific DNA endonuclease to generate a population of cleaved linear SARS-CoV-2 DNAs; (c) contacting the population of cleaved linear viral DNAs with an exonuclease to generate a population of SARS-CoV-2 deletion DNAs; (d) circularizing (e.g., via ligation) the SARS-CoV-2 deletion DNAs to generate a library of circularized SARS-CoV-2 deletion DNAs; and (e) sequencing members of the library of circularized SARS-CoV-2 deletion DNAs to identify deletion interfering particles (DIPs). In some cases, the method includes inserting a barcode sequence prior to or simultaneous with step (d).In some cases, the inserting of step (a) includes inserting a transposon cassette into the population of circular SARS-CoV-2 viral DNAs, wherein the transposon cassette includes the target sequence for the sequence specific DNA endonuclease, and where the generated population of sequence-inserted SARS-CoV-2 DNAs is a population of transposon-inserted viral DNAs. In some cases (e.g., in some cases when using a CRISPR/Cas endonuclease), a subject method does not include step (a), and the first step of the method is instead cleaving members of the library in different locations relative to one another, which step can be followed by the exonuclease step.

### B. Target Sequence and Sequence Specific DNA Endonucleases

In some cases, a target sequence for a sequence specific DNA endonuclease is inserted into a SARS-CoV-2 DNA, for example, using a transposon cassette. The ‘target sequence’ is also referred to herein as a recognition sequence or recognition site. The term sequence specific endonuclease is used herein to refer to a DNA endonuclease that binds to and/or recognizes the target sequence in a SARS-CoV-2 DNA and cleaves the SARS-CoV-2 DNA. In other words, a sequence specific DNA endonuclease recognizes a specific sequence (a recognition sequence) within a SARS-CoV-2 DNA molecule and cleaves the molecule based on that recognition. In some cases, the sequence specific DNA endonuclease cleaves the SARS-CoV-2 DNA within the recognition sequence and in some cases it cleaves outside of the recognition sequence (e.g., in the case of type IIS restriction endonucleases).

The term sequence specific DNA endonuclease encompasses can include, for example, restriction enzymes, meganucleases, and programmable genome editing nucleases. Examples of sequence specific endonucleases include but are not limited to: restriction endonucleases such as EcoRI, EcoRV, BamHI, etc.; meganucleases such as LAGLI DADG meganucleases (LMNs), 1-Scel, 1-Ceul, 1-Crel, 1-Dmol, 1-Chul, 1-Dirl, 1-Flmul, 1-Flmull, 1-Anil, 1-ScelV, 1-Csml, 1-Panl, I-Panll, 1-PanMI, 1-Scell, 1-Ppol, 1-Scelll, 1-Ltrl, 1-Gpil, 1-GZel, 1-Onul, 1-HjeMI, 1-Msol, 1-Tevl, I-Tevll, 1-Tevlll, Pi-Miel, P1-Mtul, P1-Pspl, PI-TD I, PI-TD Π, P1-SceV, and the like; and programmable gene editing endonucleases such as Zinc Finger Nucleases (ZFNs), transcription activator like effector nuclease (TALENs), and CRISPR/Cas endonucleases. In some cases, the sequence specific endonuclease of a subject composition and/or method is selected from: a meganuclease and a programmable gene editing endonuclease. In some cases, the sequence specific endonuclease of a subject composition and/or method is selected from: a meganuclease, a ZFN, a TALEN, and a CRISPR/Cas endonuclease (e.g., Cas9, Cpfl, and the like).

In some cases, the sequence specific endonuclease of a subject composition and/or method is a meganuclease. In some cases the meganuclease is selected from: LAGLIDADG meganucleases (LMNs), 1-Scel, 1-Ceul, 1-Crel, 1-Dmol, 1-Chul, 1-Dirl, 1-Flmul, 1-Flmull, 1-Anil, I-ScelV, 1-Csml, 1-Panl, 1-Panll, 1-PanMI, 1-Scell, 1-Ppol, 1-Scelll, 1-Ltrl, 1-Gpil, 1-GZel, 1-Onul, I-HjeMI, 1-Msol, 1-Tevl, 1-Tevll, 1-TevIll, Pl-Mlel, PI-Mtul, PI-Pspl, PI-Tli I, PI-Tli II, and Pl-SceV. In some cases, the meganuclease 1-Scel is used. In some cases, the meganuclease 1-Ceul is used. In some cases, the meganucleases 1-Scel and 1-Ceul are used.

In some cases, the sequence specific DNA endonuclease is a programmable genome editing nuclease. The term “programmable genome editing nuclease” is used herein to refer to endonucleases that can be targeted to different sites (recognition sequences) within a SARS-CoV-2 DNA. Examples of suitable programmable genome editing nucleases include but are not limited to zinc finger nucleases (ZFNs), TAL-effector DNA binding domain-nuclease fusion proteins (transcription activator-like effector nucleases (TALENs)), and CRISPR/Cas endonucleases (e.g., class 2 CRISPR/Cas endonucleases such as a type II, type V, or type VI CRISPR/Cas endonucleases). Thus, in some embodiments, a programmable genome editing nuclease is selected from: a ZFN, a TALEN, and a CRISPR/Cas endonuclease (e.g., a class 2 CRISPR/Cas endonuclease such as a type H, type V, or type VI CRISPR/Cas endonuclease). In some cases, the sequence specific endonuclease of a subject composition and/or method is a CRISPR/Cas endonuclease (e.g., Cas9, Cpfl, and the like). In some cases, the sequence specific endonuclease of a subject composition and/or method is selected from: a meganuclease, a ZFN, and a TALEN.

Information related to class 2 type H CRISPR/Cas endonuclease Cas9 proteins and Cas9 guide RNAs (as well as methods of their delivery) (as well as information regarding requirements related to protospacer adjacent motif (PAM) sequences present in SARS-CoV-2 nucleic acids) can be found, for example, in the following Jinek et al., Science. 2012 Aug. 17; 337(6096):816-21; Chylinski et al., RNA Biol. 2013 May; 10(5): 726-37; Ma et al., Biomed Res Int. 2013; 2013:270805; Hou et al., Proc Natl Acad Sci USA. 2013 Sep. 24; 1 10(39): 15644-9; Jinek et al., Elife. 2013; 2:e00471; Pattanayak et al., Nat Biotechnol. 2013 September; 31 (9):839-43; Qi et al, Cell. 2013 Feb. 28; 152(5): 1173-83; Wang et al., Cell. 2013 May 9; 153(4):910-8; Auer et. al., Genome Res. 2013 Oct. 31; Chen et. al., Nucleic Acids Res. 2013 Nov. 1; 41 (20):e19; Cheng et. al., Cell Res. 2013 October; 23(10): 1 163-71; Cho et. al., Genetics. 2013 November; 195(3): 1 177-80; DiCarlo et al., Nucleic Acids Res. 2013 April; 41 (7):4336-43; Dickinson et. al., Nat Methods. 2013 October; 10(10): 1028-34; Ebina et. al., Sci Rep. 2013; 3:2510; Fujii et. al, Nucleic Acids Res. 2013 Nov. 1; 41 (20):e87; Hu et. al., Cell Res. 2013 November; 23(l 1): 1322-5; Jiang et. al., Nucleic Acids Res. 2013 Nov. 1; 41 (20):e188; Larson et. al., Nat Protoc. 2013 November; 8(11):2180-96; Mali et. at., Nat Methods. 2013 October; 10(10):957-63; Nakayama et. al., Genesis. 2013 December; 51 (12):835-43; Ran et. al., Nat Protoc. 2013 November; 8(1 1):2281-308; Ran et. al., Cell. 2013 Sep. 12; 154(6): 1380-9; Upadhyay et. al., G3 (Bethesda). 2013 Dec. 9; 3(12):2233-8; Walsh et. al., Proc Natl Acad Sci USA. 2013 Sep. 24; 110(39): 15514-5; Xie et. al., Mol Plant. 2013 Oct. 9; Yang et. al., Cell. 2013 Sep. 12; 154(6): 1370-9; Briner et al., Mol Cell. 2014 Oct. 23; 56(2):333-9; and U S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety. Examples and guidance related to type V CRISPR/Cas endonucleases (e.g., Cpfl) or type VI CRISPR/Cas endonucleases and guide RNAs (as well as information regarding requirements related to protospacer adjacent motif (PAM) sequences present in SARS-CoV-2 nucleic acids) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97. Useful designer zinc finger modules include those that recognize various GNN and ANN triplets (Dreier, et al., (2001) J Biol Chem 276:29466-78; Dreier, et al., (2000) J Mol Biol 303:489-502; Liu, et al., (2002) J Biol Chem 277:3850-6), as well as those that recognize various CNN or TNN triplets (Dreier, et al., (2005) J Biol Chem 280:35588-97; Jamieson, et al., (2003) Nature Rev Drug Discov 2:361-8). See also, Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29: 183-212; Segal and Barbas, (2001) Curr Opin Biotechnol 12:632-7; Segal, et al., (2003) Biochemistry 42:2137-48; Beerii and Barbas, (2002) Nat Biotechnol 20: 135-41; Carroll, et al., (2006) Nature Protocols 1: 1329; Ordiz, et al., (2002) Proc Natl Acad Sci USA 99: 13290-5; Guan, et al., (2002) Proc Natl Acad Sci USA 99: 13296-301.

For more information on ZFNs and TALENs (as well as methods of their delivery), refer to Sanjana et al., Nat Protoc. 2012 Jan. 5; 7(1): 171-92 as well as international patent applications WO2002099084; WO00/42219; WO02/42459; WO2003062455; WO03/080809; WO05/014791; WO05/084190; WO08/021207; WO09/042186; WO09/054985; WO10/079430; and WO10/065123; U.S. Pat. Nos. 8,685,737; 6,140,466; 6,511,808; and 6,453,242; and US Patent Application Nos. 2011/0145940, 2003/0059767, and 2003/0108880; all of which are hereby incorporated by reference in their entirety.

In some cases (e.g., in the case of restriction enzymes), the recognition sequence is a constant (does not change) for the given protein (e.g., the recognition sequence for the BamHI restriction enzyme is). In some cases, the sequence specific DNA endonuclease is ‘programmable’ in the sense that the protein (or its associated RNA in the case of CRISPR/Cas endonucleases) can be modified/engineered to recognize a desired recognition sequence. In some cases (e.g., in cases where the sequence specific DNA endonuclease is a meganuclease and/or in cases where the sequence specific DNA endonuclease is a CRISPR/Cas endonuclease), the recognition sequence has a length of 14 or more nucleotides (nt) (e.g., 15 or more, 16 or more, 17 or more, 18 or more, 19 or more, or 20 or more nt). In some cases, the recognition sequence has a length in a range of from 14-40 nt (e.g., 14-35, 14-30, 14-25, 15-40, 15-35, 15-30, 15-25, 16-40, 16-35, 16-30, 16-25, 17-40, 17-35, 17-30, or 17-25 nt). In some cases, the recognition sequence has a length of 14 or more base pairs (bp) (e g., 15 or more, 16 or more, 17 or more, 18 or more, 19 or more, or 20 or more bp). In some cases, the recognition sequence has a length in a range of from 14-40 bp (e.g., 14-35, 14-30, 14-25, 15-40, 15-35, 15-30, 15-25, 16-40, 16-35, 16-30, 16-25, 17-40, 17-35, 17-30, or 17-25 bp).

When referring above to the lengths of a recognition sequence, the double-stranded helix and the recognition sequence can be thought of in terms of base pairs (bp), while in some cases (e.g., in the case of CRISPR/Cas endonucleases) the recognition sequence is recognized in single stranded form (e.g., a guide RNA of a CRISPR/Cas endonuclease can hybridize to the SARS-CoV-2 DNA) and the recognition sequence can be thought of in terms of nucleotides (nt). However, when using ‘bp’ or ‘nt’ herein when referring to a recognition sequence, this terminology is not intended to be limiting. As an example, if a particular method or composition described herein encompasses both types of sequence specific DNA endonuclease (those that recognize ‘bp’ and those that recognize ‘nt’), either of the terms ‘nt’ or ‘bp’ can be used without limiting the scope of the sequence specific DNA endonuclease, because one of ordinary skill in the art would readily understand which term (‘nt’ or ‘bp’) would appropriately apply, and would understand that this depends on which protein is chosen. In the case of a length limitation of the recognition sequence, one of ordinary skill in the art would understand that the length limitation being discussed equally applies regardless of whether the term ‘nt’ or ‘bp’ is used.

### C. Chew Back (Exonuclease Digestion)

After the circular SARS-CoV-2 DNAs are cleaved, generating a population of cleaved linear SARS-CoV-2 DNAs, the open ends of the linear SARS-CoV-2 DNAs are digested (chewed back) by exonucleases. Many different exonucleases will be known to one of ordinary skill in the art and any convenient exonuclease can be used. In some cases, a 5′ to 3′ exonuclease is used. In some cases, a 3′ to 5′ exonuclease is used. In some cases, an exonuclease is used that has both 5′ to 3′ and 3′ to 5′ exonuclease activity. In some cases, more than one exonuclease is used (e.g., 2 exonucleases). In some cases, the population of cleaved linear SARS-CoV-2 DNAs is contacted with a 5′ to 3′ exonuclease and a 3′ to 5′ exonuclease (e.g., simultaneously or one before the other).

In some cases, a T4 DNA polymerase is used as a 3′ to 5′ exonuclease (in the absence of dNTPs, T4 DNA polymerase has 3′ to 5′ exonuclease activity). In some cases, Reej is used as a 5′ to 3′ exonuclease. In some cases, T4 DNA polymerase (in the absence of dNTPs) and Reej are used. Examples of exonucleases include but are not limited to: DNA polymerase (e.g., T4 DNA polymerase) (in the absence of dNTPs), lambda exonuclease (5′->3′), T5 exonuclease (5′->3′), exonuclease II (3->5′), exonuclease V (5′->3′ and 3′->5′), T7 exonuclease (5′->3′), exonuclease T, exonuclease VII (truncated) (5′->3′), and Reej exonuclease (5′->3′).

The rate of DNA digestion (chew back) is sensitive to temperature, thus the size of the desired deletion can be controlled by regulating the temperature during exonuclease digestion. For example, in the examples section below when using T4 DNA polymerase (in the absence of dNTPs) and Reej as the exonucleases, the double-end digestion rate (chew back rate) proceeded at a rate of 50 bp/min at 37° C. and at a reduced rate at lower temperatures (e.g., as discussed in the examples section below). Thus, temperature can be decreased or increased and/or digestion time can be decreased or increased to control the size of deletion (i.e., the amount of exonuclease digestion). For example, in some cases, the temperature and time are adjusted so that exonuclease digestion causes a deletion in a desired size range. As an illustrative example, if a deletion in a range of from 500-1000 base pairs (bp) is desired, the time and temperature of digestion can be adjusted so that 250-500 nucleotides are removed from each end of the linearized (cut) SARS-CoV-2 DNA, i.e., the size of the deletion is the sum of the number of nucleotides removed from each end of the linearized SARS-CoV-2 DNA. In some cases, the temperature and time are adjusted so that exonuclease digestion causes a deletion having a size in a range of from 20-1000 bp (e.g., from 20-50, 40-80, 20-100, 40-100, 20-200, 40-200, 60-100, 60-200, 80-150, 80-250, 100-250, 150-350, 100-500, 200-500, 200-700, 300-800, 400-800, 500-1000, 700-1000, 20-800, 50-1000, 100-1000, 250-1000, 50-1000, 50-750, 100-1000, or 100-750 bp).

In some cases, contacting with an exonuclease (one or more exonucleases) is performed at a temperature in a range of from room temperature (e.g., 25° C.) to 40° C. (e.g., from 25-37° C., 30-37° C., 32-40° C., or 30-40° C.). In some cases, contacting with an exonuclease is performed at 37° C. In some cases, contacting with an exonuclease is performed at 32° C. In some cases, contacting with an exonuclease is performed at 30° C. In some cases, contacting with an exonuclease is performed at 25° C. In some cases, contacting with an exonuclease is performed at room temperature. In some cases, the SARS-CoV-2 DNA is contacted with an exonuclease (one or more exonucleases) for a period of time in a range of from 10 seconds to 40 minutes (e.g., from 10 seconds to 30 minutes, 10 seconds to 20 minutes, 10 seconds to 15 minutes, 10 seconds to 10 minutes, 30 seconds to 30 minutes, 30 seconds to 20 minutes, 30 seconds to 15 minutes, 30 seconds to 12 minutes, 30 seconds to 10 minutes, 1 to 40 minutes, 1 to 30 minutes, 1 to 20 minutes, 1 to 15 minutes, 1 to 10 minutes, 3 to 40 minutes, 3 to 30 minutes, 3 to 20 minutes, 3 to 15 minutes, 3 to 12 minutes, or 3 to 10 minutes). In some cases, the contacting is for a period of time in a range of from 20 seconds to 15 minutes.

After DNA digestion (chew back), the remaining overhanging DNA ends can be repaired (e.g., using T4 DNA Polymerase plus dNTPs) or in some cases the single stranded overhangs can be removed (e.g., using a nuclease such as mung bean nuclease that cleaves single stranded DNA but not double stranded DNA). For example, if only a 5′ to 3′ or 3′ to 5′ exonuclease is used, a nuclease specific for single stranded DNA (i.e., that does not cut double stranded DNA) (e.g., mung bean nuclease) can be used to remove the overhang.

The step of contacting with one or more exonucleases (i.e., chew back) can be carried out in the presence or absence of a single strand binding protein (SSB protein). An SSB is a protein that binds to exposed single stranded DNA ends, which can achieve numerous results, including but not limited to: (i) helping stabilize the DNA by preventing nucleases from accessing the DNA, and (ii) preventing hairpin formation within the single stranded DNA. Examples of SSB proteins include but are not limited to a eukaryotic SSB protein (e.g., replication protein A (RPA)); bacterial SSB protein; and viral SSB proteins. In some cases, the step of contacting with one or more exonucleases is performed in the presence of an SSB. In some cases, the step of contacting with one or more exonucleases is performed in the absence of an SSB.

### D. Barcode

In some embodiments, the members of a library are ‘tagged’ by adding a barcode to the SARS-CoV-2 DNAs after exonuclease digestion (and after remaining overhanging DNA ends are repaired/removed). The addition of a barcode can be performed prior to or simultaneously with re-circularizing (ligation). As used herein, term “barcode” is used to mean a stretch of nucleotides having a sequence that uniquely tags members of the library for future identification. For example, in some cases, a barcode cassette (from a pool of random barcode cassettes) can be added and the library sequenced so that it is known which barcode sequence is associated with which particular member, i.e., with which particular deletion (e.g., a lookup table can be created such that each member of a deletion library has a unique barcode). In this way, members of a deletion library can be tracked and accounted for by virtue of presence of the barcode (instead of having to identify the members by determining the location of deletion). Identifying the presence of a short stretch of nucleotides using any convenient assay can easily be accomplished. Use of such barcodes is easier than isolating and sequencing individual members (in order to determine location of deletion) each time the library is used for a given experiment. For example, one can readily determine which library members are present before an experiment (e.g., before introducing library members into cells to assay for viral infectivity), and compare this to which members are present after the experiment by simply assaying for the presence of the barcode before and after, e.g., using high throughput sequencing, a microarray, PCR, qPCR, or any other method that can detect the presence/absence of a barcode sequence.

In some cases, a barcode is added as a cassette. A barcode cassette is a stretch of nucleotides that have at least one constant region (a region shared by all members receiving the cassette) and a barcode region (i.e., a barcode sequence—a region unique to the members that receive the barcode such that the barcode uniquely marks the members of the library). For example, a barcode cassette can include (i) a constant region that is a primer site, which site is in common among the barcode cassettes used, and (ii) a barcode sequence that is a unique tag, e.g., can be a stretch of random sequence. In some cases, a barcode cassette includes a barcode region flanked by two constant regions (e.g., two different primer sites). As an illustrative example, in some cases a barcode cassette is a 60 bp cassette that includes a 20 bp random barcode flanked by 20 bp primer binding sites (e.g., see FIG. 4).

A barcode sequence can have any convenient length and is preferably long enough so that it uniquely marks the members of a given library of interest. In some cases, the barcode sequence has a length of from 15 bp to 40 bp (e.g., from 15-35 bp, 15-30 bp, 15-25 bp, 17-40 bp, 17-35 bp, 17-30 bp, or 17-25 bp). In some cases, the barcode sequence has a length of 20 bp. Likewise, a barcode cassette can have any convenient length, and this length depends on the length of the barcode sequence plus the length of the constant region(s). In some cases, the barcode cassette has a length of from 40 bp to 100 bp (e.g., from 40-80 bp, 45-100 bp, 45-80 bp, 45-70 bp, 50-100 bp, 50-80 bp, or 50-70 bp). In some cases, the barcode cassette has a length of 60 bp.

A barcode or barcode cassette can be added using any convenient method. For example, a linear SARS-CoV-2 DNA can be recircularized by ligation to a 3′-dT-tailed barcode cassette drawn from a pool of random barcode cassettes. The nicked hemiligation product can then be sealed and transformed into a host cell, e.g., a bacterial cell.

### E. Generating a Product

In some cases, a subject method includes a step of generating (e.g., from a generated library of circularized SARS-CoV-2 deletion DNAs) at least one of: linear double stranded DNA (dsDNA) products (e.g., via cleavage of the circular DNA, via PCR, etc.), linear single stranded DNA (ssDNA) products (e.g. , via transcription and reverse transcription), linear single stranded RNA (ssRNA) products (e.g., via transcription), and linear double stranded RNA (dsRNA) products. If so desired, the linear SARS-CoV-2 products can then be introduced into a cell (e.g., mammalian cell). For example, a common technique for RNA viruses is to perform in vitro transcription from a dsDNA template (circular or linear) to make RNA, and then to introduce this RNA into cells (e.g., via electroporation, chemical methods, etc.) to generate viral stocks.

Also, within the scope of the disclosure are kits. For example, in some cases a subject kit can include one or more of (in any combination): (i) a population of circular SARS-CoV-2 DNAs as described herein, (ii) a transposon cassette as described herein, (iii) a sequence specific DNA endonuclease as described herein, (iv) one or more guide RNAs for a CRISPR/Cas endonuclease as described herein, (v) a population of barcodes and/or barcode cassettes as described herein, and (vi) a population of host cells, e.g., for propagation of the library, for assaying for viral infectivity, etc., as described herein. In some cases, a subject kit can include instructions for use. Kits typically include a label indicating the intended use of the contents of the kit. The term label includes any writing, or recorded material supplied on or with the kit, or which otherwise accompanies the kit.

### F. Optimization and Further Development of SARS-CoV-2 TIPs

Viral load reduction of SARS-CoV-2 TIPs, in some embodiments, may be enhanced by engineering to optimize TIP transmission (ρ) and interference (ψ) parameters. The parameter “ρ” helps reduce the viral load by spreading the TIP to more cells in the tissue. Since the TIP requires wild-type virus to mobilize, if ψ is too large (generating too much inhibition), less virus is available to mobilize the TIP. Therefore, ρ and ψ generate a type of synergistic effect at the whole tissue scale.

In some embodiments, the SARS-CoV-2 TIP is evaluated for therapeutic efficacy based on ρ and ψ values. In some embodiments, mathematically modeled ρ and ψ values are used to determine whether a candidate TIP will successfully compete with SARS-CoV-2. In some embodiments, the TIP is optimized by enhancing ρ. In some embodiments, ρ is enhanced via addition of viral packaging signals. In some embodiments, the SARS-CoV-2 TIP is optimized by enhancing ψ.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Generating SARS-CoV-2 Random Deletion Libraries (RDLs)

To systematically identify regions of SARS-CoV-2 required for efficient mobilization, a randomized deletion screen was utilized similar to that described by Weinberger and Notion (2017), which created and index random-deletion libraries of HIVNL4-3.

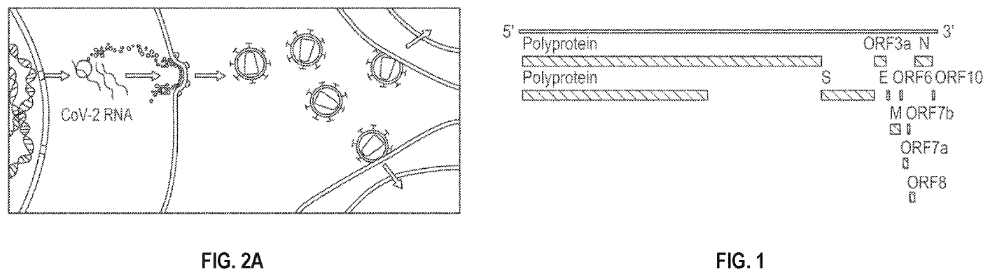

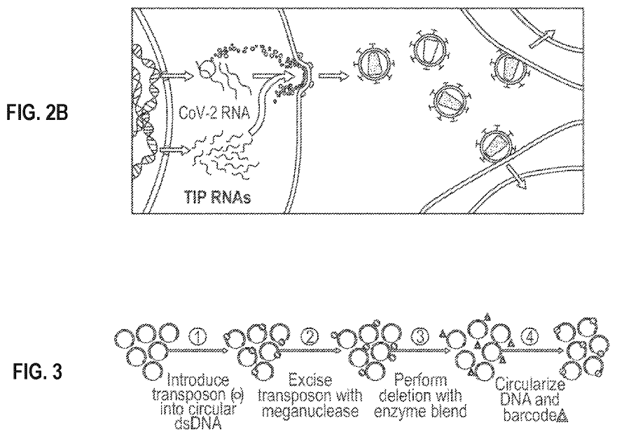

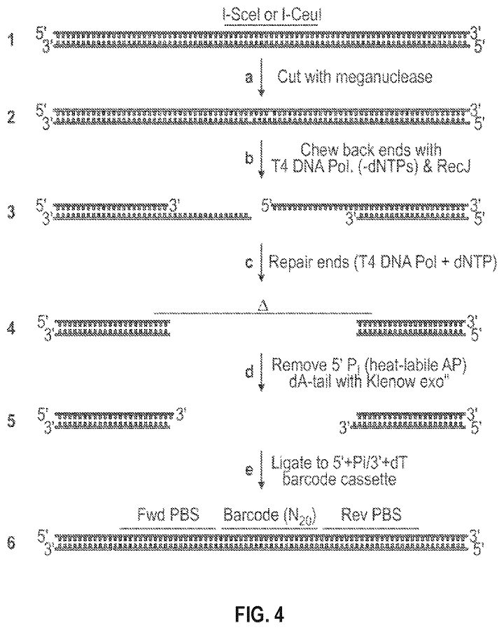

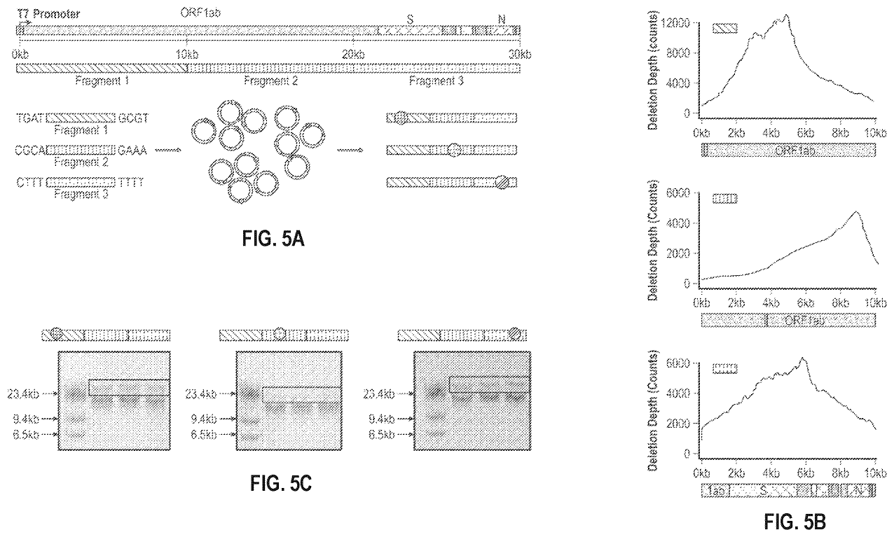

Briefly, plasmid DNA was subjected to transposon-mediated random insertion, followed by excision of the transposon and exonuclease-mediated digestion of the exposed ends to create deletions centered at a random genetic position, each of variable size. The plasmid was then re-ligated together with a cassette containing a 20-nucleotide random DNA barcode to ‘index’ the deletion. Indexing allows a deleted region to be easily identified (by the junction of genomic sequence and the barcode) and tracked/quantified by deep sequencing. This process is schematically illustrated in FIGS. 1-4. FIG. 5A further illustrates this process.

The deletion sites in the members of the libraries were sequenced. Deletion depth plots illustrated in FIG. 5B show that the sub-libraries contained over 587,000 deletions. The sub-libraries were ligated to form full-length libraries, the SARS-CoV-2 inserts were in vitro transcribed into RNA and the RNA was transfected into VeroE6 cells. The transfected cells were then infected with wild-type SARS-CoV-2 virus to test for mobilization of the deletion mutants. After three vims passages, RNA was extracted from cells and the presence of deletion barcodes was analyzed.

### SARS-CoV-2 Viroreactor

A SARS-CoV-2 viroreactor was set up using VeroE6 cells growing on silicone beads in suspension that can be infected with the SARS-CoV-2 deletion mutants, thereby creating a dynamic system to improve infection and ultimately evolution of SARS-CoV-2 therapeutic interfering particles (TIPs). The conditions used for the SARS-CoV-2 viroreactor were adapted from the protocol used to isolate an HIV TIP (described by Weinberger and Notion (2017)).

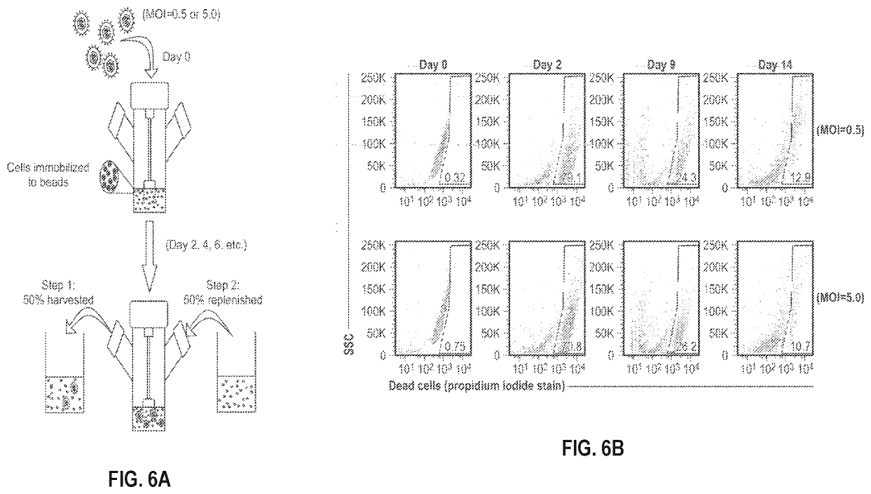

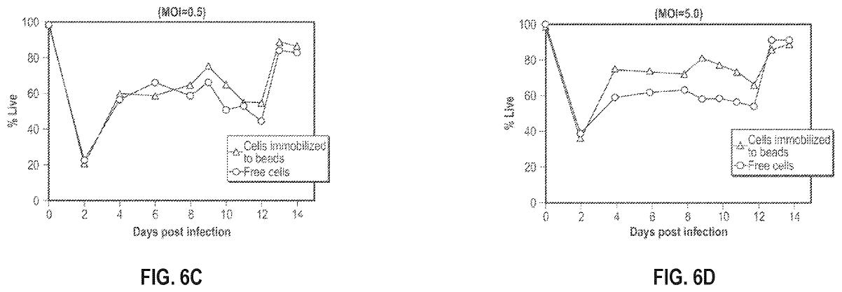

As illustrated in FIG. 6A, when the VeroE6 cells reached steady-state density, they were infected with the SARS-CoV-2 deletion mutants at a MOI of either 0.5 or 5, under gentle agitation. Half of the culture was removed from the reactor every day and replaced with fresh cells and media. Samples removed from the reactor were centrifugated, supernatants were frozen for later analysis and cell viability was measured by flow cytometry using a propidium iodine staining protocol (FIG. 6B). Cell viability was low (35-60%) at 2 days post infection (dpi) (FIGS. 6C-6D) but started recovering as soon as 4 days post-infection (dpi) and stayed stable (60-805) until 12 dpi. At day 13, the cultures recovered to over 90% of cell viability.

### Example 2: SARS-CoV-2 Therapeutic Interfering Particles (TIPs)

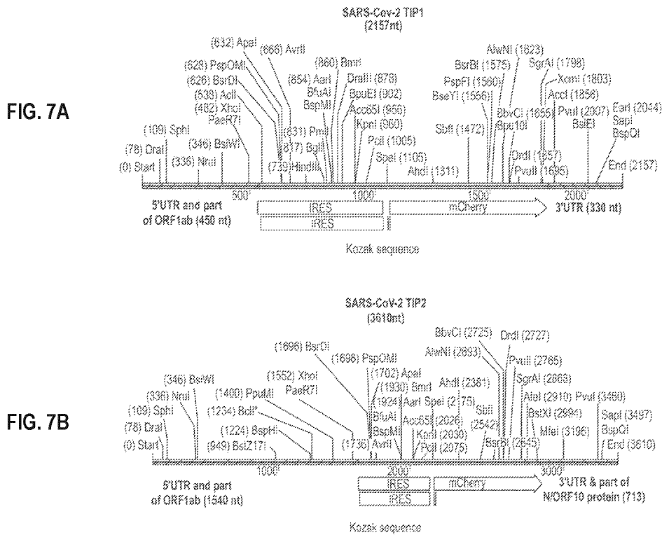

Minimal TIP sub-genomic synthetic constructs, TIP1 and TIP2, with the structures shown in FIGS. 7A-7B were designed and cloned. The TIP1 and TIP2 constructs encode varying portions of the 5′ and 3′UTRs of SARS-CoV-2 and express an mCherry reporter protein driven from an IRES. The plasmid constructs were sequence verified.

More specifically, the TIPs encompass stem loop 5 in the 5′UTR which encodes a predicted packaging signal, as well as the entirety of the 3′UTR and a 1280 nucleotide (nt) reporter cassette encoding an internal ribosome entry sequence (IRES) driving expression of a fluorescent reporter protein (mCherry). TIP1 (˜2.1 kb) encodes the first 450 nts of the 5′UTR plus part of polyprotein ORF1ab and the last 328 nts of the 3′UTR plus the reporter cassette, whereas TIP2 (˜3.5 kb) encodes 1540 nts encompassing the 5′UTR and part of ORF1ab and the last 713 nts of the genome containing part of N protein, ORF 10, and the 3′UTR, along with the reporter cassette. All TIP and control mRNAs were in vitro transcribed and a 5′ methyl cap and ˜100-nt 3′ polyA tail were added following in vitro transcription.

The 5′ SARS-CoV-2 sequences in TIP1 are as shown below (SEQ ID NO: 28).

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | CGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 411 | GCGTTTTGCC |

The 3′ SARS-CoV-2 sequences in TIP1 are shown below as SEQ ID NO: 29.

| 1 | GACCACACAA GGCAGATGGG CTATATAAAC GTTTTCGCTT |
|---|---|
| 41 | TTCCGTTTAC GATATATAGT CTACTCTTGT GCAGAATGAA |
| 81 | TTCTCGTAAC TACATAGCAC AAGTAGATGT AGTTAACTTT |
| 121 | AATCTCACAT AGCAATCTTT AATCAGTGTG TAACATTAGG |
| 161 | GAGGACTTGA AAGAGCCACC ACATTTTCAC CGAGGCCACG |
| 201 | CGGAGTACGA TCGAGTGTAC AGTGAACAAT GCTAGGGAGA |
| 241 | GCTGCCTATA TGGAAGAGCC CTAATGTGTA AAATTAATTT |
| 281 | TACTAGTGCT ATCCCCATGT GATTTTAATA GCTTCTTAGG |
| 321 | AGAATGACAA AAAAAAAAAA AAAAAAAAAA AAAAAAAAAA |
| 361 | A |
| The 5′ SARS-CoV-2 sequences in TIP2 are as shown below (SEQ ID NO: 30). | |
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTACGTTT |
| 241 | CGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 441 | GCGTTTTGCC TCAACTTGAA CAGCCCTATG TGTTCATCAA |
| 481 | ACGTTCGGAT GCTCGAACTG CACCTCATGG TCATGTTATG |
| 521 | GTTGAGCTGG TAGCAGAACT CGAAGGCATT CAGTACGGTC |
| 561 | GTAGTGGTGA GACACTTGGT GTCCTTGTCC CTCATGTGGG |
| 601 | CGAAATACCA GTGGCTTACC GCAAGGTTCT TCTTCGTAAG |
| 641 | AACGGTAATA AAGGAGCTGG TGGCCATAGT TACGGCGCCG |
| 681 | ATCTAAAGTC ATTTGACTTA GGCGACGAGC TTGGCACTGA |
| 721 | TCCTTATGAA GATTTTCAAG AAAACTGGAA CACTAAACAT |
| 761 | AGCAGTGGTG TTACCCGTGA ACTCATGCGT GAGCTTAACG |
| 801 | GAGGGGCATA CACTCGCTAT GTCGATAACA ACTTCTGTGG |
| 841 | CCCTGATGGC TACCCTCTTG AGTGCATTAA AGACCTTCTA |
| 881 | GCACGTGCTG GTAAAGCTTC ATGCACTTTG TCCGAACAAC |
| 921 | TGGACTTTAT TGACACTAAG AGGGGTGTAT ACTGCTGCCG |
| 961 | TGAACATGAG CATGAAATTG CTTGGTACAC GGAACGTTCT |
| 1001 | GAAAAGAGCT ATGAATTGCA GACACCTTTT GAAATTAAAT |
| 1041 | TGGCAAAGAA ATTTGACACC TTCAATGGGG AATGTCCAAA |
| 1081 | TTTTGTATTT CCCTTAAATT CCATAATCAA GACTATTCAA |
| 1121 | CCAAGGGTTG AAAAGAAAAA GCTTGATGGC TTTATGGGTA |
| 1161 | GAATTCGATC TGTCTATCCA GTTGCGTCAC CAAATGAATG |
| 1201 | CAACCAAATG TGCCTTTCAA CTCTCATGAA GTGTGATCAT |
| 1241 | TGTGGTGAAA CTTCATGGCA GACGGGCGAT TTTGTTAAAG |
| 1281 | CCACTTGCGA ATTTTGTGGC ACTGAGAATT TGACTAAAGA |
| 1321 | AGGTGCCACT ACTTGTGGTT ACTTACCCCA AAATGCTGTT |
| 1361 | GTTAAAATTT ATTGTCCAGC ATGTCACAAT TCAGAAGTAG |
| 1401 | GACCTGAGCA TACTCTTGCC GAATACCATA ATGAATCTCC |
| 1441 | CTTGAAAACC ATTCTTCGTA AGGGTGGTCG CACTATTGCC |
| 1481 | TTTGGAGGCT GTGTGTTCTC TTATGTTGGT TGCCATAACA |
| 1521 | AGTGTGCCTA TTGGGTTCCA gaattagatc tctcgaggtt |
| 1561 | aacgaattct gctatacgaa gttatccatc |

The 3′ SARS-CoV-2 sequences in TIP2 are as shown below (SEQ ID NO: 31).

| | |
|---|---|
| 1 | ATTTGCCCCC AGCGCTTCAG CGTTCTTCGG AATGTCGCGG |
| 41 | ATTGGCATGG AAGTCACACC TTCGGGAACG TGGTTGACCT |
| 81 | ACACAGGTGC CATCAAATTG GATGACAAAG ATCCAAATTT |
| 121 | CAAAGATCAA GTCATTTTGC TGAATAAGCA TATTGACGCA |
| 161 | TACAAAACAT TCCCACCAAC AGAGCCTAAA AAGGACAAAA |
| 201 | AGAAGAAGGC TGATGAAACT CAAGCCTTAC CGCAGAGACA |
| 241 | GAAGAAACAG CAAACTGTGA CTCTTCTTCC TGCTGCAGAT |
| 281 | TTGGATGATT TCTCCAAACA ATTGCAACAA TCCATGAGCA |
| 321 | GTGCTGACTC AACTCAGGCC TAAACTCATG CAGACCACAC |
| 361 | AAGGCAGATG GGCTATATAA ACGTTTTCGC TTTTCCGTTT |
| 401 | ACGATATATA GTCTACTCTT GTGCAGAATG AATTCTCGTA |
| 441 | ACTACATAGC ACAAGTAGAT GTAGTTAACT TTAATCTCAC |
| 481 | ATAGCAATCT TTAATCAGTG TGTAACATTA GGGAGGACTT |
| 521 | GAAAGAGCCA CCACATTTTC ACCGAGGCCA CGCGGAGTAC |
| 561 | GATCGAGTGT ACAGTGAACA ATGCTAGGGA GAGCTGCCTA |
| 601 | TATGGAAGAG CCCTAATGTG TAAAATTAAT TTTAGTAGTG |
| 641 | CTATCCCCAT GTGATTTTAA TAGCTTCTTA GGAGAATGAC |
| 681 | AAAAAAAAAA AAAAAAAAAA AAAAAAAAAA AAA |

Two additional TIP variants were also cloned TIP1* and TIP2*, these contain the common C-241-T mutation within the 5′UTR. This C241T UTR mutation co-transmits across populations together with the spike protein D614G mutation.

Hence, the 5′ SARS-CoV-2 sequences in TIP1* are as shown below (SEQ ID NO: 32).

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | TGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 411 | GCGTTTTGCC |

Similarly, the 5′ SARS-CoV-2 sequences in TIP2* are as shown below (SEQ ID NO: 33).

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | TGTCCSGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 441 | GCGTTTTGCC TCAACTTGAA CAGCCCTATG TGTTCATCAA |
| 481 | ACGTTCGGAT GCTCGAACTG CACCTCATGG TCATGTTATG |
| 521 | GTTGAGCTGG TAGCAGAACT CGAAGGCATT CAGTACGGTC |
| 561 | GTAGTGGTGA GACACTTGGT GTCCTTGTCC CTCATGTGGG |
| 601 | CGAAATACCA GTGGCTTACC GCAAGGTTCT TCTTCGTAAG |
| 641 | AACGGTAATA AAGGAGCTGG TGGCCATAGT TACGGCGCCG |
| 681 | ATCTAAAGTC ATTTGACTTA GGCGACGAGC TTGGCACTGA |
| 721 | TCCTTATGAA GATTTTCAAG AAAACTGGAA CACTAAACAT |
| 761 | AGCAGTGGTG TTACCCGTGA ACTCATGCGT GAGCTTAACG |
| 801 | GAGGGGCATA CACTCGCTAT GTCGATAACA ACTTCTGTGG |
| 841 | CCCTGATGGC TACCCTCTTG AGTGCATTAA AGACCTTCTA |
| 881 | GCACGTGCTG GTAAAGCTTC ATGCACTTTG TCCGAACAAC |
| 921 | TGGACTTTAT TGACACTAAG AGGGGTGTAT ACTGCTGCCG |
| 961 | TGAACATGAG CATGAAATTG CTTGGTACAC GGAACGTTCT |
| 1001 | GAAAAGAGCT ATGAATTGCA GACACCTTTT GAAATTAAAT |
| 1041 | TGGCAAAGAA ATTTGACACC TTCAATGGGG AATGTCCAAA |
| 1081 | TTTTGTATTT CCCTTAAATT CCATAATCAA GACTATTCAA |
| 1121 | CCAAGGGTTG AAAAGAAAAA GCTTGATGGC TTTATGGGTA |
| 1161 | GAATTCGATC TGTCTATCCA GTTGCGTCAC CAAATGAATG |
| 1201 | CAACCAAATG TGCCTTTCAA CTCTCATGAA GTGTGATCAT |
| 1241 | TGTGGTGAAA CTTCATGGCA GACGGGCGAT TTTGTTAAAG |
| 1281 | CCACTTGCGA ATTTTGTGGC ACTGAGAATT TGACTAAAGA |
| 1321 | AGGTGCCACT ACTTGTGGTT ACTTACCCCA AAATGCTGTT |
| 1361 | GTTAAAATTT ATTGTCCAGC ATGTCACAAT TCAGAAGTAG |
| 1401 | GACCTGAGCA TAGTCTTGCC GAATACCATA ATGAATCTGG |
| 1441 | CTTGAAAACC ATTCTTCGTA AGGGGGTCG CACTATTGCC |
| 1481 | TTTGGAGGCT GTGTGTTCTC TTATGTTGGT TGCCATAACA |
| 1521 | AGTGTGCCTA TTGGGTTCCA gaattagatc tctcgaggtt |
| 1561 | aacgaattct gctatacgaa gttatccctc |

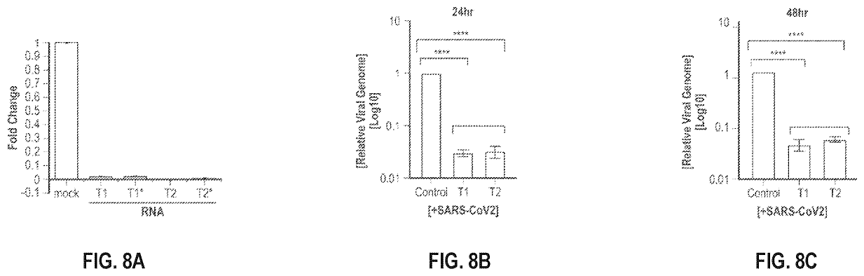

To test whether TIP constructs can reduce SARS-CoV-2 replication, mRNA from the four TIP constructs was generated by in vitro transcription from a T7 promoter operably linked upstream of the TIP in each plasmid. The different preparations of in vitro transcribed TIP mRNA were transfected into Vero E6 cells (TIP1, TIP1*, TIP2, or TIP2*), and the cells were infected with SARS-CoV-2 (WA strain) at an MOI=0.005. At 48 hrs post-infection samples were harvested and a yield-reduction assay was conducted (see FIG. 8). Yield-reduction assays were measured by fold-reduction in SARS-CoV-2 mRNA (E gene) at 48 hrs post infection because the SARS-CoV-2 E (envelope) gene does not occur in the TIP sequences. As shown in FIG. 8, all of the TIP constructs reduced SARS-CoV-2 viral replication, but the TIP2 construct exhibited the greatest interference with SARS-CoV-2.

### Example 3: SARS-CoV-2 TIPs are Mobilized by SARS-CoV-2 and Transmit Together with SARS-CoV-2

Supernatant transfer experiments were performed to test the ability of the candidate TIPs to be mobilized by SARS-CoV-2 and transmitted together with SARS-CoV-2.

SARS-CoV-2-infected Vero E6 cells were transfected with various TIP candidates having the structures shown in FIGS. 7A-7B. Analysis for mCherry expression could therefore be used as a measure of TIP replication. Supernatant was collected from this first population of cells at 96 hours post-infection and the supernatant was transferred to a second population of fresh Vero cells. As a first control, supernatant was transferred from naive uninfected cells to Vero cells, and as a second control supernatant was transferred from SARS-CoV-2 infected cells that were not transfected with TIPs. Flow cytometry was performed to analyze mCherry expression of the second population of cells at 48 hours after supernatant transfer.

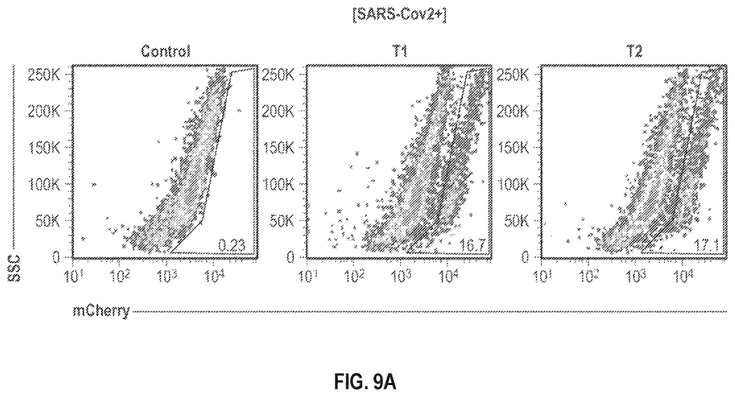

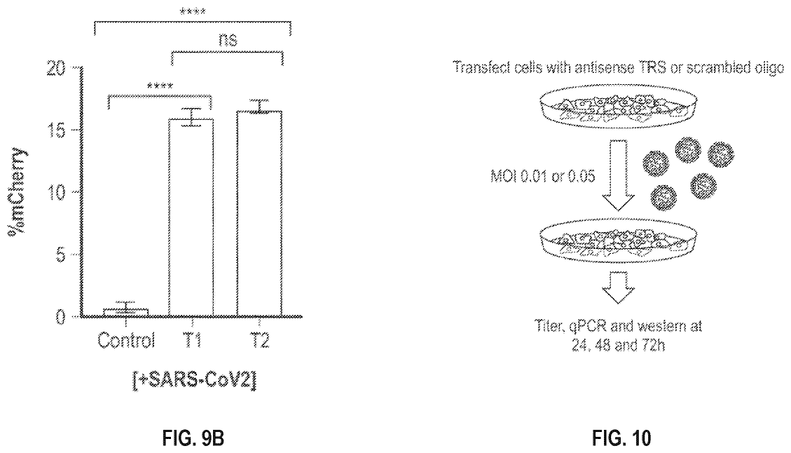

As shown in FIG. 9, the first and second controls showed no mCherry expression (FIGS. 9A-9B). However, the supernatant from cells transfected with TIP candidate mRNA and infected with SARS-CoV-2 did generate mCherry producing cells, indicating that functional viral-like particles (VLPs) were being generated by SARS-CoV-2 helper virus (FIGS. 9C-9I). In general, we found that mRNA transfection yielded better mobilization (FIGS. 9G-9H) than DNA transfection (FIGS. 9C-9F). This was consistent with results from the yield reduction assay by RT-qPCR where mRNA transfection also yielded better interference with SARS-CoV-2 than did DNA transfection (not shown).

### Example 4: Transcription Regulating Sequences (TRS) for Antiviral Intervention Against SARS-CoV-2

This Example describes use of antisense RNAs to intervene or interfere with SARS-CoV-2 infection.

Transcription initiation is regulated in coronaviruses by several types of consensus transcription regulating sequences (TRSs): TRS1-L: 5′-cuaaac-3′ (SEQ ID NO: 36), TRS2-L: 5′-acgaac-3′ (SEQ ID NO: 37), and TRS3-L, 5′-cuaaacgaac-3′ (SEQ ID NO: 38).

To evaluate whether transcription can be inhibited from these transcriptional initiation sites, the following antisense TRS RNAs were developed:

TRS1-
(SEQ ID NO: 25)
ACGAACCUAAACACGAACCUAAAC;
TRS2-
(SEQ ID NO: 26)
(ACGAACACGAACACGAACACGAAC;
and
TRS3-
(SEQ ID NO: 27)
CUAAACCUAAACCUAAACCUAAAC.

Vero cells were transfected with the antisense TRS RNAs and then infected with SARS-CoV-2 (MOI 0.01 or 0.05). As controls, cells were transfected with a scrambled RNA (instead of a TRS RNA) and then infected with SARS-CoV-2 (MOI 0.01 or 0.05).

The titers of SARS-CoV-2 were determined by quantitative PCR and western blots were prepared at 24, 48, and 72 hours.

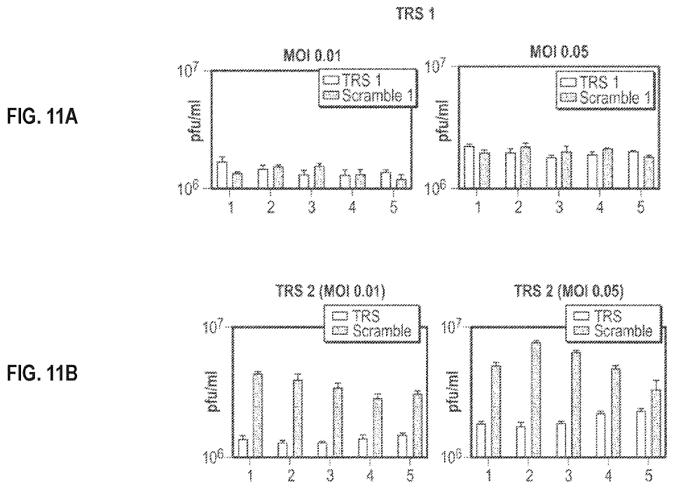

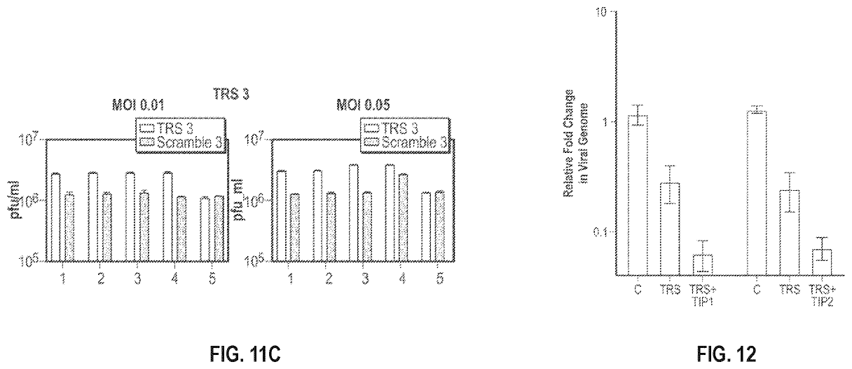

As shown in FIGS. 11A-11C, use of the TRS2 antisense reduced SARS-CoV-2 titers to the greatest extent (FIG. 11B).

Vero cells were then incubated with combination of a TRS2 antisense with either TIP1 or TIP2, and then the cells were infected with SARS-CoV-2. The fold changes in SARS-CoV-2 genome numbers were then determined.

As shown in FIG. 12, the combination of the TRS2 antisense with either the TIP1 or TIP2 significantly reduced the SARS-CoV-2 genome numbers compared to the TRS alone.

### Example 5: SARS-CoV-2 TIPs Reduce Replication of Different SARS-CoV-2 Strains

This Example describes use of therapeutic interfering particles (TIP1 and TIP2) to intervene or interfere with different SARS-CoV-2 strains.

Vero cells were pretreated with lipid nanoparticles encapsulating therapeutic interfering particles (TIP1 or TIP2 at 0.3 ng/μL or 0.003 ng/μL), or a control RNA. At two hours post-treatment the cells were infected (MOI 0.005) with one of the following SARS-CoV-2 strains:
The 501Y.V2.HV variant of SARS-CoV-2, colloquially known as a South African variant;
The 501 Y.V2.HV delta variant of SARS-CoV-2, colloquially known as a South African variant; and
The B.1.1.7 variant, colloquially known as a U.K. variant.

Supernatant from the infected cultures was harvested at 48 hours post-infection and the SARS-CoV-2 viral titer was quantified.

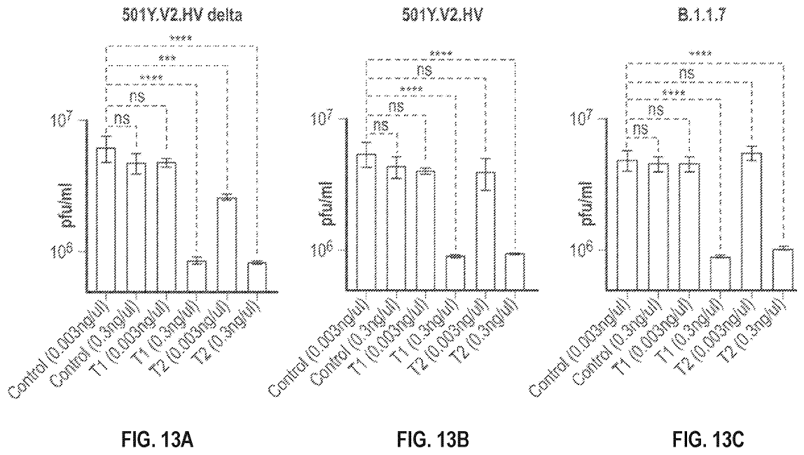

FIGS. 13A-13C illustrate that TIP and TIP2 significantly reduce the replication of SARS-CoV-2 in a dose-dependent manner.

### Example 6: SARS-CoV-2 TIPs Inhibit SARS-CoV-2 in Primary Human Lung Organoids

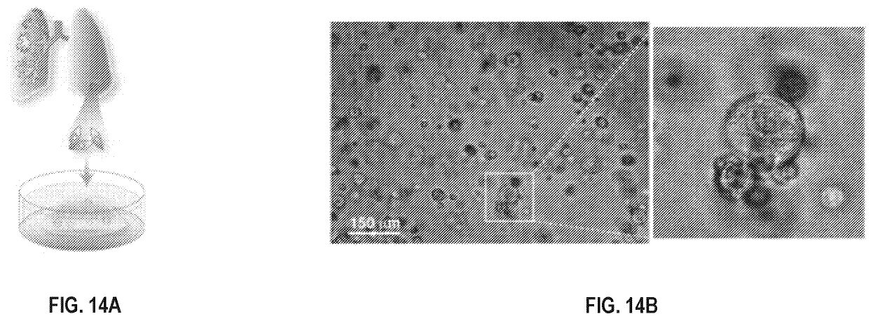

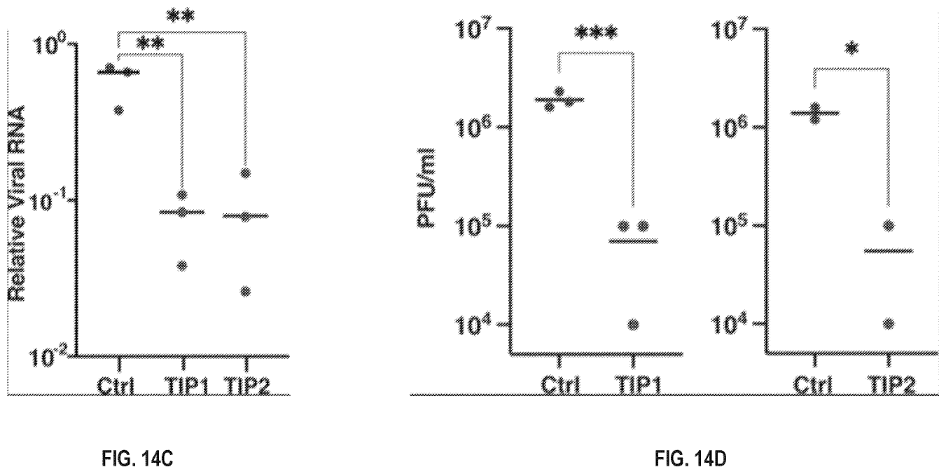

To test if TIPs interfered with SARS-CoV-2 in a more physiological setting, a human lung organoid model as shown in FIG. 14A was employed. Organoids were established and characterized using primary human small-airway epithelial cells (FIG. 14B), obtained from three donors. The organoids were transfected with either TIP1, TIP2, or an RNA control, and then infected with SARS-CoV-2 virus at MOI=0.5 24 hrs later.

Viral titers in lung organoids were assayed by RT-qPCR 24 hrs post infection. Briefly, at indicated time points, SARS-CoV-2 infected cells were lysed in TRIzol LS (Invitrogen) solution. RNA was extracted using the Direct-zol™ RNA extraction kit (Zymo Research Inc.), and treated with DNase. 1 μg of RNA was used for each reverse transcriptase reaction, and cDNA was analyzed by quantitative real-time polymerase chain reaction (qRT-PCR) analysis using SYBR green PCR master mix (Thermofisher Scientific) with sequence specific primers. All the qRT-PCR measurements were normalized to GAPDH or β-actin.

Viral titers in lung organoids were additionally assayed by plaque forming unit (PFU) analysis. Briefly, cells were prepared by plating as a confluent monolayer 24 hrs before performing the plaque assay. On the day of the plaque assay, media was aspirated, cells were washed with PBS, and 250 μL of diluted virus in modified DMEM media (DMEM, 2% FBS, L-glut, P/S) was added to the confluent monolayer, followed by incubation at 37° C. for 1 hr with gentle rocking every 15 mins. After one hr of incubation, 2 mL of overlay media (1.2% Avicel in 1×MEM) was added to each well. At 72 hrs post infection, overlay media was aspirated, monolayer was washed with PBS, and fixed with 10% formalin for 1 hr. Plaques were stained with 0.1% crystal violet for 10 ms and washed with cell culture grade water three times, followed by enumeration of plaques using ImageJ and viral titer calculation to pfu/mL.

Both RT-qPCR (FIG. 14C) and PFU analysis (FIG. 14D) confirmed that TIPs reduced SARS-CoV-2 by ˜1-Log compared to Ctrl RNA.

### Example 7: TIPs Generate Virus-Like Particles (VLPs), Compete for Viral Trans Elements, and Mobilize with R₀>1

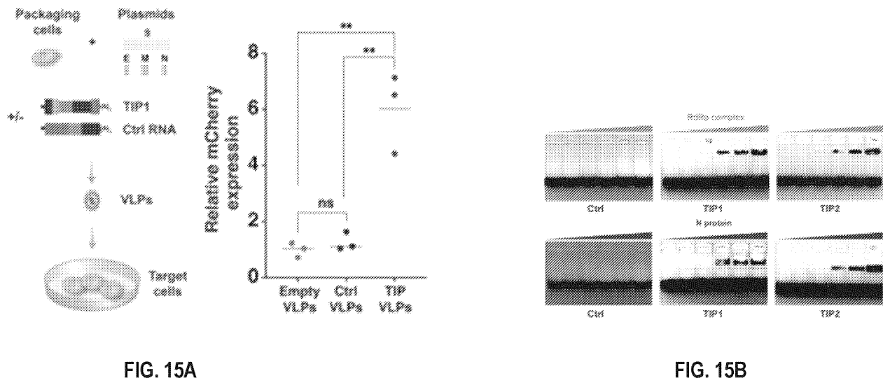

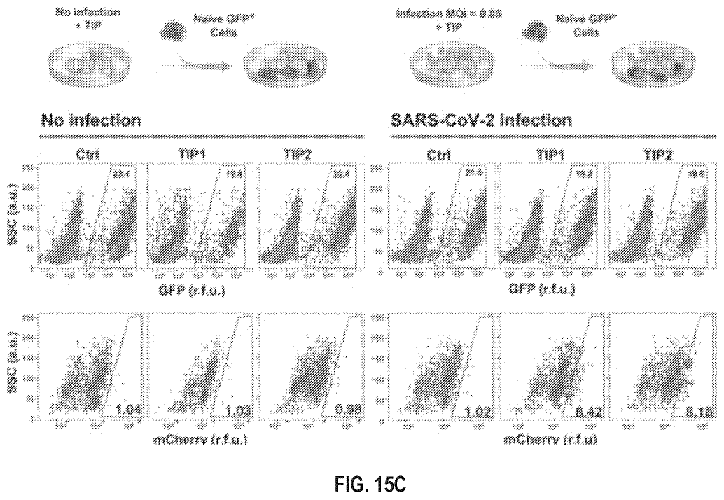

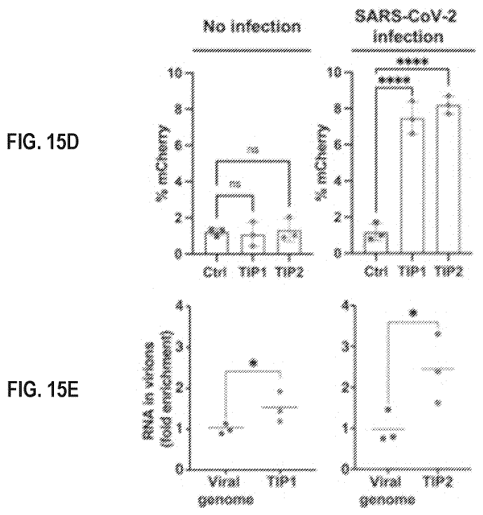

To determine if TIP RNAs are packaged into VLPs, reconstitution assays were performed (FIG. 15A). Cells were co-transfected with expression vectors each encoding a cDNA for the matrix (M), envelope (E), spike (S), or nucleocapsid (N) protein of SARS-CoV-2 together with TIP RNA, Ctrl RNA, or no RNA. Supernatant was concentrated (ultracentrifuged) and imaged for presence of VLPs by transmission electron microscopy (EM) and, in parallel, analyzed for functional VLP transduction of naive cells. EM analysis showed the presence of abundant ˜100 nm-diameter VLPs. RT-qPCR for mCherry showed substantial TIP transduction of naive cells when VLPs where reconstituted using TIP RNA but not Ctrl RNA (FIG. 15A).

To test if TIP mRNAs directly bind and compete for SARS-CoV-2 viral proteins, electromobility shift assays (EMSA) were performed on TIP mRNA and viral proteins. EMSA analysis of cell extracts expressing either RdRp complex or N protein, incubated with purified TIP1 or TIP2 RNA, showed that TIP RNAs bind both RdRp complex and N proteins, whereas Ctrl RNA does not bind either of these proteins (FIG. 15B).

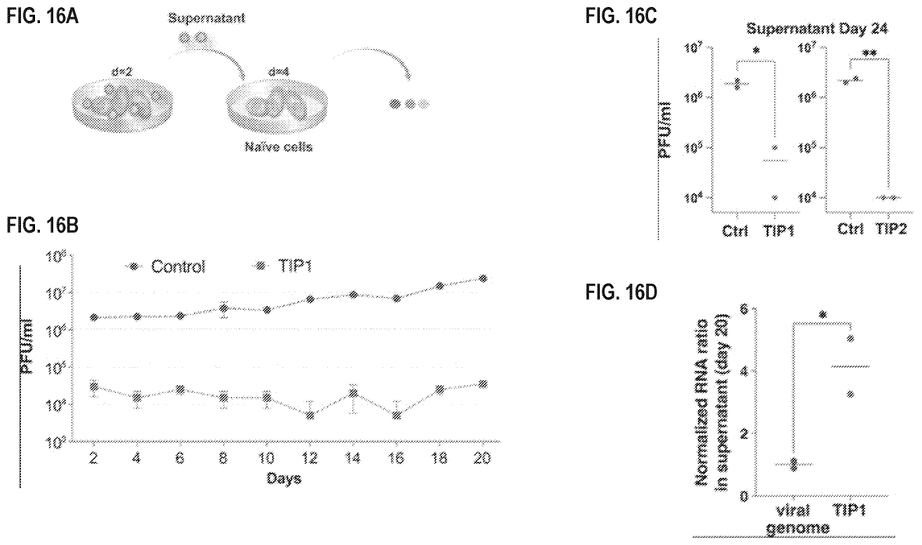

To quantify the R₀ of TIPs in the context of SARS-CoV-2 infection, the supernatant-transfer assay was modified into a ‘1^{st} round supernatant transfer assay’. TIP-transfected cells were infected at a low MOI (MOI=0.05), washed to remove virus, and at two hours post infection GFP+ reporter cells were introduced to the culture (at ˜20% of total cells). TIP mobilization into reporter cells was quantified using the percentage of mCherry+ cells within the GFP+ population at 12 hrs post infection. Infection-dependent mobilization was confirmed by comparing to uninfected samples for all RNAs (FIGS. 15C-15D), and the control RNAs did not mobilize either in the absence or presence of virus, with the exception of 5′UTR which carries the putative packaging signal. The fraction of TIP+ cells, approximately 8%, was corrected for background autofluorescence, to yield 6.3% TIP+ cells (as compared to approximately 5% infected cells for the original SARS-CoV-2 infection at MOI=0.05) and this translated to 4% infected cells after accounting for the addition of 20% GFP+ cells in the assay. TIPs propagating into 6.3% of new cells from the initial wild-type infection of 4% of cells represents a roughly 50% increase or roughly an R₀=1.57; for comparison, R₀=2 would require a doubling, from 4% to 8%, of cells being mCherry+. This R₀>1 finding for TIPs is further verified in the below Examples using a continuous serial-passage approach (see FIGS. 16A-16D).

To verify that TIP RNA was packaged into virions at a high level, the relative fraction of TIP RNA versus SARS-CoV-2 genomic RNA was quantified in virions isolated from supernatant by RT-qPCR (FIG. 15E). Analysis showed that the TIP RNA was significantly enriched (1.5-2 fold) compared to SARS-CoV-2 viral genomes.

Overall, these data indicate the TIPs do not restrict viral entry or early viral expression (i.e., via induction of a cellular response), that TIP RNA generates functional TIP VLPs in the presence of M, N, E, and S, that TIP RNAs bind to, and may compete for SARS-CoV-2 proteins in cells, and that competition for packaging and replication resources is sufficient to quantitatively account for the measured TIP-mediated yield reduction.

### Example 8: SARS-CoV-2 TIPs Exhibit a High Barrier to Evolution of Resistance

Long-term virus cultures were established, where viral supernatant was continually serially passaged to new naive cells to sustain high-level viral infection and selected for viral escape mutants (FIG. 16A). The continuous viral cultures were initiated using cells transfected with either Ctrl RNA or TIP1 RNA, and cells were then infected and viral supernatant serially passaged onto naive, non-transfected cells every 48 hours for ˜3 weeks, with virus titered at each passage.

SARS-CoV-2 replicative fitness was enhanced by ˜1-Log over 3 weeks in the Ctrl RNA continuous culture (FIG. 16B). In contrast, the continuous cultures initiated in the presence of TIP RNA exhibited an immediate ˜2-Log decrease in viral titer by PFU (FIG. 16B), consistent with single-round yield reduction data (FIGS. 14A-14E). This reduction in viral titer was sustained over the course of the 20-day culture.

To verify that this viral load reduction in the continuous culture was due to TIP interference and not a cellular peculiarity, supernatant from a parallel control culture after day 20 was used to infect cells in the presence of TIP RNA, and the 2-Log decrease in viral titer was recapitulated (FIG. 16C). RT-qPCR analysis of the culture supernatants indicated that TIP RNA exhibited a 4-fold increase relative to SARS-CoV-2 RNA on day 20 (FIG. 16D). These continuous culture data indicate conditional amplification and sustained transmission of the TIP, i.e., R₀>1, since the TIP RNA was only added to the infected culture once (i.e., a single administration on day 0).

### Example 9: Intranasal SARS-CoV-2 TIP Delivery Inhibits SARS-CoV-2, Reduces Pro-Inflammatory Cytokines, and Prevents Pulmonary Edema in Hamsters

A Syrian Golden Hamster model of SARS-CoV-2 infection (Sia et al., 2020) was used to assay the in vivo efficacy of SARS-CoV-2 TIPs.

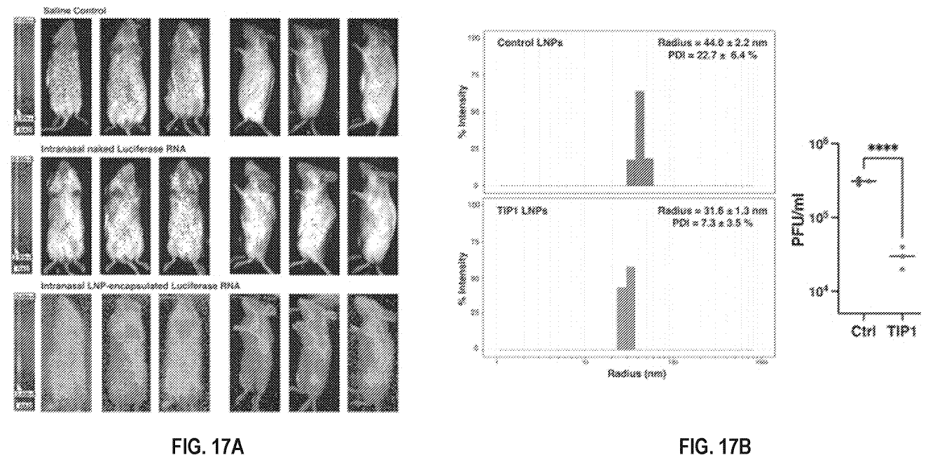

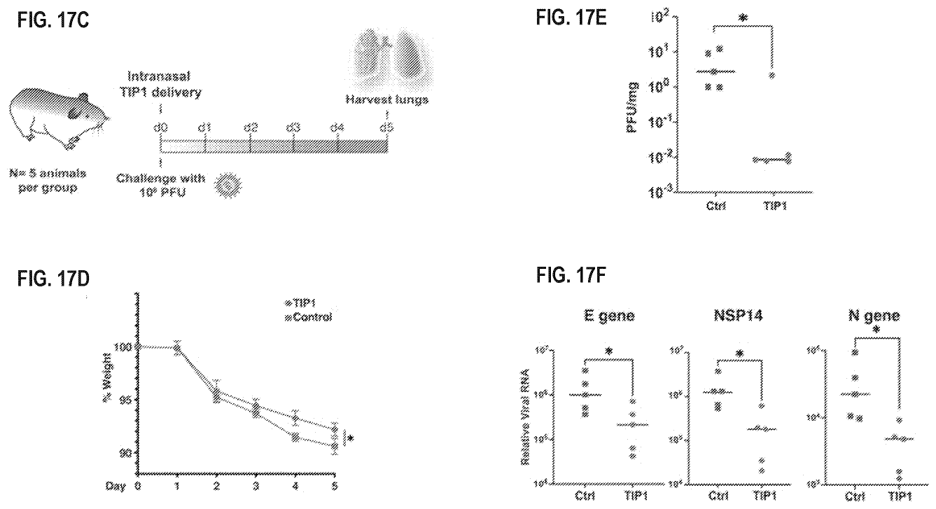

Intranasal administration of various RNA delivery approaches were tested for their ability to efficiently deliver RNA to the respiratory tract of rodents. Using an in vitro transcribed luciferase-expressing RNA, purified RNA alone (‘naked RNA’), RNA encapsulated into cationic polymer nanocarriers (i.e., polyethylenimine), and RNA encapsulated in lipid nanoparticles (LNPs), were tested. LNPs exhibited efficient in vivo RNA delivery to the lungs after intranasal administration (FIG. 17A). LNPs containing either TIP1 RNA or Ctrl RNA were generated and characterized. LNP-encapsulated TIP RNA retained antiviral efficacy using yield-reduction assays in Vero cells (FIG. 17B).

Next, the TIP or Ctrl RNA LNPs were administered intranasally to Syrian Golden hamsters and were challenged with SARS-CoV-2 (10⁶ PFUs) (FIG. 17C). Control-treated hamsters showed weight loss following infection, but this was significantly ameliorated by TIP treatment (FIG. 17D). Analysis of infectious virus in lung tissue harvested on day 5 from hamsters confirmed a significant ˜2-Log reduction in SARS-CoV-2 viral load in TIP-treated animals (FIG. 17E). One animal did not exhibit a reduction in viral load which may be consistent with inefficient TIP dosing/delivery. RT-qPCR analysis of viral transcripts in the lung exhibited a correlated, but lesser, 1-Log reduction in viral load for TIP-treated animals (FIG. 17F).

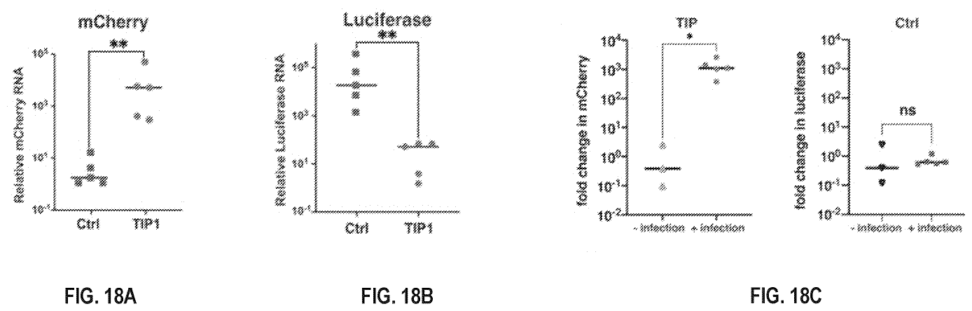

To determine if conditional propagation of TIPs correlated with SARS-CoV-2 inhibition in vivo, TIP expression was analyzed in the lungs on day 5 by RT-qPCR. High levels of TIP RNA were observed (FIG. 18A), whereas Ctrl RNA on day 5 was present at substantially lower levels (FIG. 18B). Moreover, to confirm that presence of SARS-CoV-2 infection is obligatory for conditional propagation of TIPs, the amount of TIP or Ctrl RNA in the presence vs. absence of virus on day 5 in hamster lungs was determined. Ctrl RNA levels in the lungs were unaffected by SARS-CoV-2 infection, in contrast with TIP RNA that was significantly amplified by 4 Logs in the presence of SARS-CoV-2 infection (FIG. 18C). All RT-qPCR threshold cycle (Ct) values for luciferase in the TIP-treated animals and mCherry in the control animals were >30, indicating negligible non-specific amplification.

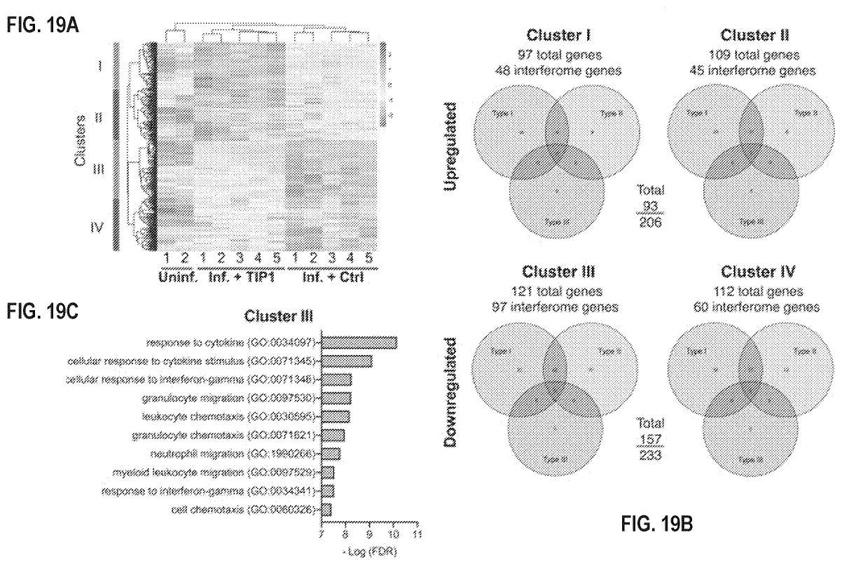

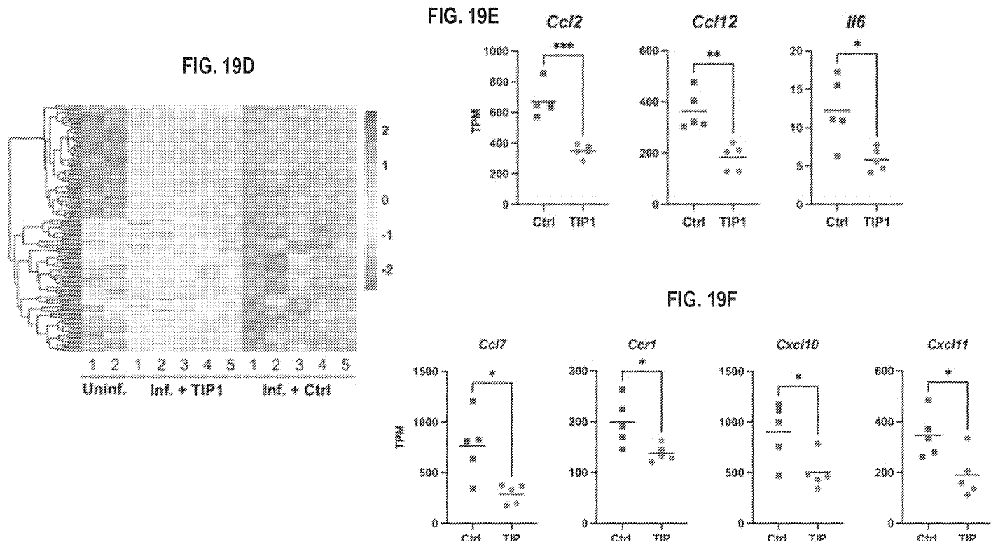

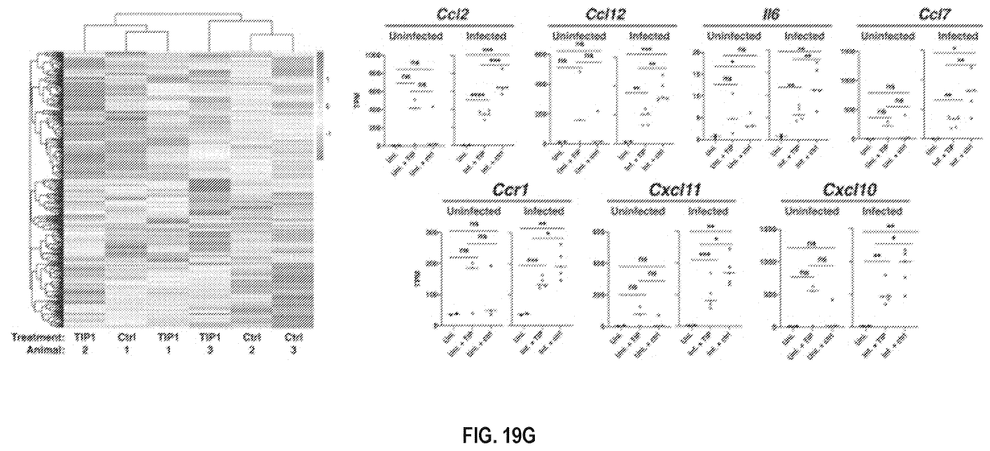

Cytokine and interferon responses in the lungs of infected animals was analyzed by performing RNA sequencing (RNAseq). Analysis of hamster lung samples showed that TIP-treated animals could be clearly differentiated from control-treated animals, with 206 upregulated genes and 233 downregulated genes (FIG. 19A). These differentially expressed genes (DEGs) form four clusters when analyzed together with uninfected hamster lung samples (FIG. 19A). The majority of downregulated genes in TIP-treated animals were interferon-stimulated genes (ISGs) (157 out of 233; FIG. 19B), especially for genes in cluster III (97 out of 121; FIG. 19B). Gene ontology (GO) analysis showed that TIP treatment significantly downregulated pro-inflammatory immune response pathways, which are significantly enriched in cluster III (FIG. 19C). The reduced expression of cluster III genes in TIP-treated samples (FIG. 19D) suggested alleviated immune responses. Specifically, expression levels of proinflammatory cytokines and receptors previously reported to be upregulated in COVID-19 patients—including 116, Ccl2, Ccl7, Cxcl10, Ccr1—were significantly reduced in TIP-treated animals (FIG. 19E and FIG. 19F). Importantly, DEGs that can distinguish TIP-treated from Ctrl-treated in infected animals cannot separate TIP from control in uninfected animals (FIG. 19A vs. FIG. 19G), indicating the alleviated proinflammatory immune response is infection-dependent and not solely due to TIP RNAs.

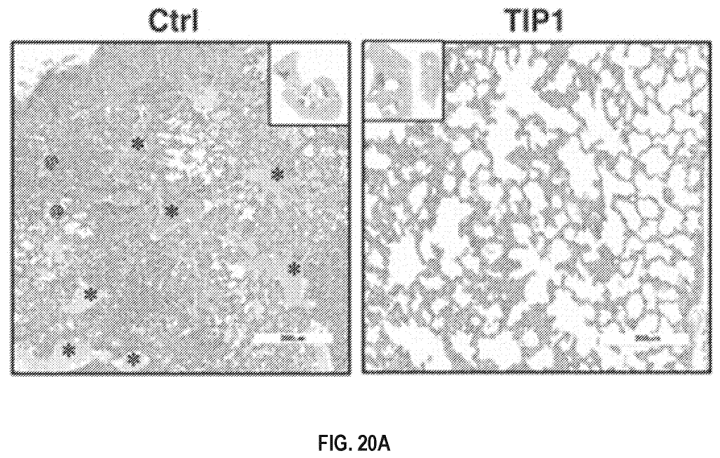

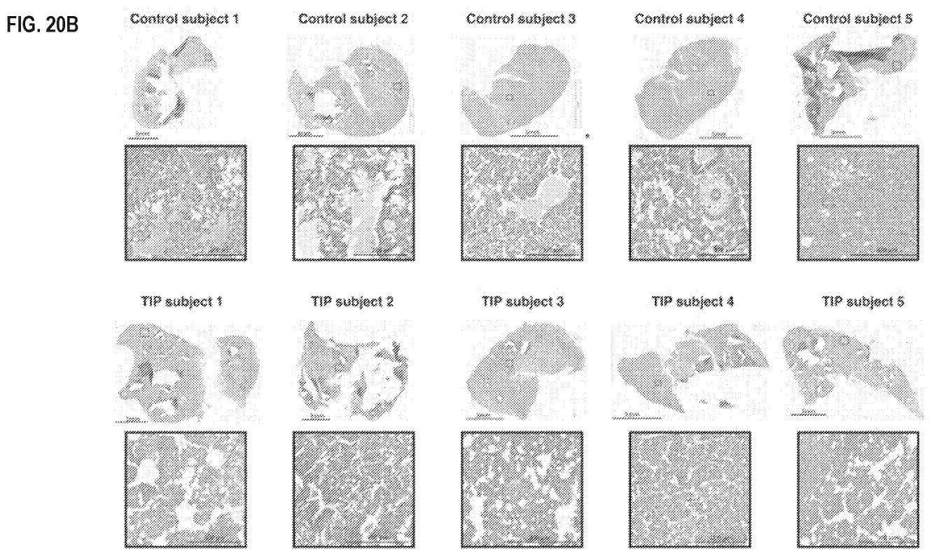

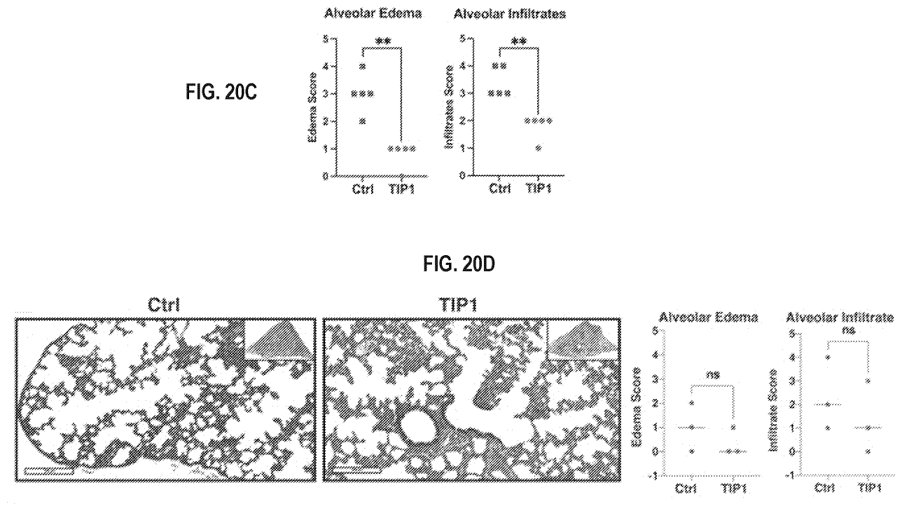

A histological analysis of day 5 hamster lung tissue samples was performed. Control animals exhibiting signs of severe pulmonary edema not present in TIP-treated animals (FIG. 20A). Specifically, despite all animals exhibiting some signs of inflammation consistent with infection, control animals evidenced pronounced alveolar edema and conspicuous cell infiltrates in alveolar spaces (FIG. 20A), indicating vascular leakage. Lungs of TIP-treated animals showed substantially less edema and cell infiltration. Histopathological scoring of the images (FIG. 20B) indicated significant reductions in alveolar edema and cell infiltrates in the TIP-treated hamsters (FIG. 20C). Uninfected hamsters treated with either TIP or Ctrl RNA LNPs were used as controls, and showed non-significant difference in the alveolar edema and infiltrates, confirming the severe vascular leakage is due to viral infection (FIG. 20D).

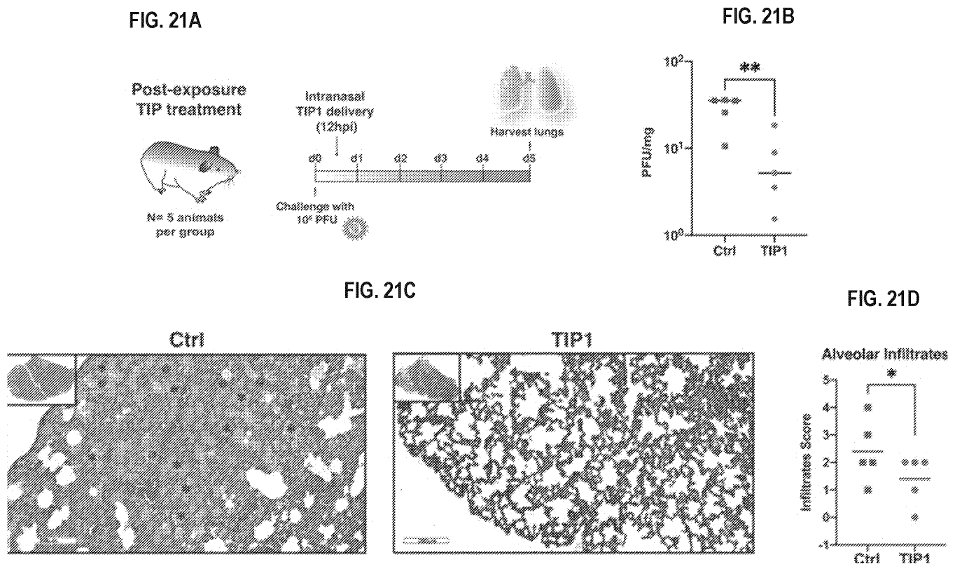

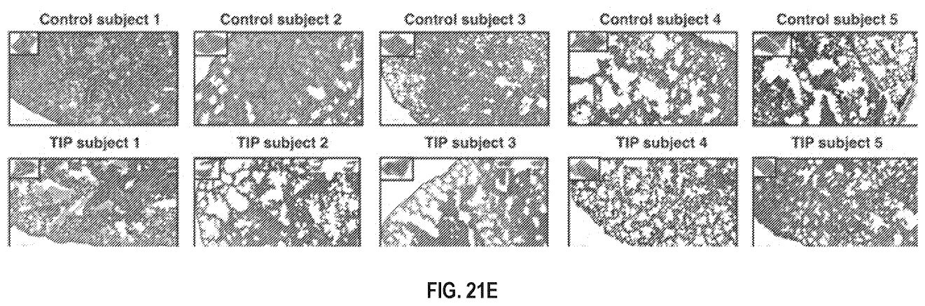

To test the efficacy of TIPs in a post-exposure therapeutic setting, hamsters were inoculated with SARS-CoV-2 (10⁶ PFUs) and then given a single intranasal administration of LNP TIP or LNP Ctrl RNA at 12 hrs post infection (FIG. 21A). In agreement with the above results, a significant reduction in SARS-CoV-2 viral load (FIG. 21B) as well as reduced pathogenesis in the lungs of animals at day 5 (FIGS. 21C-21E) was observed.

1. A recombinant SARS-CoV-2 construct capable of interfering with SARS-CoV-2 replication, comprising:
(a) a 5′ untranslated region (UTR) region comprising at least 100 nucleotides of a SARS-CoV-2 5′UTR or a variant thereof,
(b) an intervening sequence, and
(c) a 3′UTR region comprising at least 100 nucleotides of a SARS-CoV-2 3′UTR or a variant thereof,
wherein the recombinant SARS-CoV-2 construct cannot replicate by itself,
wherein the recombinant SARS-CoV-2 construct can replicate in the presence of SARS-CoV-2, and
wherein the intervening sequence is about 1 base pair (bpl to about 29000 bp in length.

2. The recombinant SARS-CoV-2 construct of claim 1, wherein the total length of the 5′UTR region, the intervening sequence, and the 3′UTR region in the recombinant SARS-CoV-2 construct is about 1000 bp to about 10000 bp.

3. (canceled)

4. The recombinant SARS-CoV-2 construct of claim 1, wherein the 5′UTR region comprises nucleotides 1-265 of SEQ ID NO:1, or a variant thereof.

5. The recombinant SARS-CoV-2 construct claim 1, wherein:
(i) the 5′UTR region comprises two or more copies of 5′UTR sequences, each comprising at least 100 nucleotides of a SARS-CoV-2 5′UTR or a variant thereof; and/or
(ii) the 3′UTR region comprises two or more copies of 3′UTR sequences, each comprising at least 100 nucleotides of a SARS-CoV-2 3′UTR or a variant thereof.

6. The recombinant SARS-CoV-2 construct of claim 1, wherein the 3′UTR region comprises nucleotides 29675-29870 of SEQ ID NO:1, or a variant thereof.

7. The recombinant SARS-CoV-2 construct of claim 6, wherein the 3′UTR region comprises nucleotides 29675-29903 of SEQ ID NO:1, or a variant thereof.

8. (canceled)

9. The recombinant SARS-CoV-2 construct of claim 1, further comprising a packaging signal for SAR-CoV-2.

10. (canceled)

11. The recombinant SARS-CoV-2 construct of claim 1, wherein the intervening sequence comprises a SARS-CoV-2 sequence, a heterologous sequence, or a combination thereof.

12-13. (canceled)

14. The recombinant SARS-CoV-2 construct of claim 1, wherein the recombinant SARS-CoV-2 construct comprises;
(i) nucleotides 1-450 of SEQ ID NO:1, or a variant thereof;
(ii) nucleotides 1-1540 of SEQ ID NO:1, or a variant thereof;
(iii) nucleotides 29543-29903 of SEQ ID NO:1, or a variant thereof; or
(iv) nucleotides 29191-29903 of SEQ ID NO:1, or a variant thereof.

15-18. (canceled)

19. The recombinant SARS-CoV-2 construct of claim 11, wherein the intervening sequence comprises a heterologous sequence, and wherein the heterologous sequence does not encode a functional protein.

20. The recombinant SARS-CoV-2 construct of claim 11, wherein the intervening sequence comprises a heterologous sequence, and wherein the heterologous sequence encodes one or more functional proteins.

21-23. (canceled)

24. The recombinant SARS-CoV-2 construct of claim 1, wherein the recombinant SARS-CoV-2 construct is an mRNA or a DNA.

25-35. (canceled)

36. The recombinant SARS-Cov-2 construct of claim 1, wherein the recombinant SARS-CoV-2 construct genomic RNA is produced at a higher rate than SARS-CoV-2 genomic RNA when present in a host cell infected with SARS-CoV-2, such that the ratio of the construct SAR-CoV-2 genomic RNA to the SARS-CoV-2 genomic RNA is greater than 1 in the cell.

37-39. (canceled)

40. The recombinant SARS-CoV-2 construct of claim 1, wherein the recombinant SARS-CoV-2 construct has a basic reproduction ratio (R₀)>1.

41. A viral-like particle comprising the recombinant SARS-CoV-2 construct of claim 1 and a viral envelope protein.

42. An isolated cell comprising the recombinant SARS-CoV-2 construct of claim 1.

43. A pharmaceutical composition comprising the recombinant SARS-CoV-2 construct of claim 1 and a pharmaceutically acceptable excipient.

44-46. (canceled)

47. A method of treating or preventing SARS-CoV-2 infection in an individual, comprising administering to the individual an effective amount of the pharmaceutical composition of claim 43.

48-57. (canceled)

58. An inhibitor of SARS-CoV-2 transcription regulating sequences (TRSs) that can bind to one or more of: TRS1-L: 5′-cuaaac-3′ (SEQ ID NO:36), TRS2-L: 5′-acgaac-3′ (SEQ ID NO:37), TRS3-L: 5′-cuaaacgaac-3′ (SEQ ID NO:38), or a combination thereof.

59-60. (canceled)

61. A pharmaceutical composition comprising a pharmaceutically acceptable excipient, (a) an inhibitor of SARS-CoV-2 transcription regulating sequences (TRSs) that can bind to one of more of: TRS1-L: 5′-cuaaac-3′ (SEQ ID NO:36), TRS2-L: 5′—acgaac-3′ (SEQ ID NO:37), TRS3-L, 5′-cuaaacgaac-3′ (SEQ ID NO:38), or a combination thereof; and (b) a recombinant SARS-CoV-2 construct, the construct comprising: at least 100 nucleotides of a SARS-CoV-2 5′ untranslated region (5′UTR), at least 100 nucleotides of a SARS-CoV-2 3′ untranslated region (3′UTR), or a combination thereof.


SEQUENCE LISTING (38 sequences)

SEQ ID NO: 1 - DNA, 29903 bp, Artificial Sequence
  Synthetic Construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 4405 aa, Artificial Sequence [sequence omitted]

SEQ ID NO: 3 - protein, 932 aa, Artificial Sequence
  Synthetic Construct
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 4 - protein, 601 aa, Artificial Sequence
  Synthetic Construct
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 5 - protein, 1273 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 254 aa, Artificial Sequence
  Synthetic Construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 7 - protein, 610 aa, Artificial Sequence
  Synthetic Construct
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLH

SEQ ID NO: 8 - protein, 1945 aa, Artificial Sequence
  Synthetic Construct
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 9 - protein, 500 aa, Artificial Sequence
  Synthetic Construct
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 10 - protein, 275 aa, Artificial Sequence
  Synthetic Construct
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 11 - protein, 75 aa, Artificial Sequence
  Synthetic Construct
MYSFVSEETGTLIVNSVLLFLAFVVFLLVTLAILTALRLCAYCCNIVNVSLVKPSFYVYSRVKNLNSSRVPDLLV

SEQ ID NO: 12 - protein, 222 aa, Artificial Sequence
  Synthetic Construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 13 - protein, 61 aa, Artificial Sequence
  Synthetic Construct
MFHLVDFQVTIAEILLIIMRTFKVSIWNLDYIINLIIKNLSKSLTENKYSQLDEEQPMEID

SEQ ID NO: 14 - protein, 121 aa, Artificial Sequence
  Synthetic Construct
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 15 - protein, 43 aa, Artificial Sequence
  Synthetic Construct
MIELSLIDFYLCFLAFLLFLVLIMLIIFWFSLELQDHNETCHA

SEQ ID NO: 16 - protein, 121 aa, Artificial Sequence
  Synthetic Construct
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 17 - protein, 419 aa, Artificial Sequence
  Synthetic Construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 18 - DNA, 12 bp, Artificial Sequence
  Synthetic Construct
aaatttcccggg

SEQ ID NO: 19 - protein, 38 aa, Artificial Sequence
  Synthetic Construct
MGYINVFAFPFTIYSLLLCRMNSRNYIAQVDVVNFNLT

SEQ ID NO: 20 - DNA, 36 bp, Artificial Sequence
  Synthetic Construct
ttgtgcagaatgaattctcgtaactacatagcacaa

SEQ ID NO: 21 - DNA, 29 bp, Artificial Sequence
  Synthetic Construct
taactacatagcacaagtagatgtagtta

SEQ ID NO: 22 - protein, 113 aa, Artificial Sequence
  Synthetic Construct
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 23 - DNA, 12 bp, Artificial Sequence
  Synthetic Construct
aaatttcccggg

SEQ ID NO: 24 - DNA, 12 bp, Artificial Sequence
  Synthetic Construct
aaatttcccggg

SEQ ID NO: 25 - RNA, 24 nt, Artificial Sequence
  Synthetic Construct
acgaaccuaaacacgaaccuaaac

SEQ ID NO: 26 - RNA, 24 nt, Artificial Sequence
  Synthetic Construct
acgaacacgaacacgaacacgaac

SEQ ID NO: 27 - RNA, 24 nt, Artificial Sequence
  Synthetic Construct
cuaaaccuaaaccuaaaccuaaac

SEQ ID NO: 28 - DNA, 12 bp, Artificial Sequence
  Synthetic Construct
aaatttcccggg

SEQ ID NO: 29 - DNA, 361 bp, Artificial Sequence
  Synthetic Construct
gaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggacgacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 30 - DNA, 1560 bp, Artificial Sequence
  Synthetic Construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgagttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccagaattagatctctcgaggtt

SEQ ID NO: 31 - DNA, 713 bp, Artificial Sequence
  Synthetic Construct
atttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 32 - DNA, 450 bp, Artificial Sequence
  Synthetic Construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcc

SEQ ID NO: 33 - DNA, 1590 bp, Artificial Sequence
  Synthetic Construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccagaattagatctctcgaggttaacgaattctgctatacgaagttatccctc

SEQ ID NO: 34 - DNA, 12 bp, Artificial Sequence
  Synthetic Construct
aaatttcccggg

SEQ ID NO: 35 - DNA, 12 bp, Artificial Sequence
  Synthetic Construct
aaatttcccggg

SEQ ID NO: 36 - RNA, 6 nt, Artificial Sequence
  Synthetic Construct
cuaaac

SEQ ID NO: 37 - RNA, 6 nt, Artificial Sequence
  Synthetic Construct
acgaac

SEQ ID NO: 38 - RNA, 10 nt, Artificial Sequence
  Synthetic Construct
cuaaacgaac